(19) United States
(12) Patent Application Publication
(10) Pub. No.: US 2024/0018124 A1
(43) Pub. Date: Jan. 18, 2024

(54) MODULATORS OF FPR1 AND METHODS OF USING THE SAME

(71) Applicant: BIOFRONT LTD, Grand Cayman (KY)

(72) Inventors: Tianwei MA, Beijing (CN); Feng SHI, Beijing (CN); Lichao FANG, Beijing (CN); Zheng HUANG, Beijing (CN); Hua Viola LIN, Beijing (CN); Yue XIAO, Beijing (CN)

(21) Appl. No.: 18/252,098

(22) PCT Filed: Mar. 17, 2022
(86) PCT No.: PCT/CN2022/081416

§ 371 Date: May 8, 2023

(30) Foreign Application Priority Data
Mar. 17, 2021 (WO) PCT/CN2021/081363

(51) Int. Cl. C07D 403/04; C07D 231/42; C07D 405/04; C07D 413/04

(52) U.S. Cl. CPC C07D 403/04; C07D 231/42; C07D 405/04; C07D 413/04

(57) ABSTRACT

Compounds of Formula I, compositions comprising the same, and methods of using the same, including use in treating diseases, disorders of conditions mediated by the signaling of formyl peptide receptor 1 (FPR1).

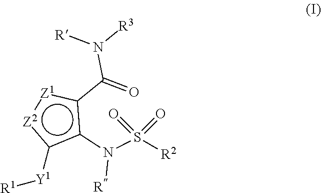

### FIELD OF THE INVENTION

This disclosure relates to compounds that are useful for the treatment of diseases. More specifically, this disclosure relates to compounds that bind to formyl peptide receptors (FPR), such as FPR1, to modulate their activities in order to reduce or eliminate disproportionate FPR-mediated signaling, which underlies the pathogenesis for an array of diseases, including, for example, diseases or disorders of the central nervous system (CNS) such as stroke, traumatic brain injury (TBI), glioblastomas and malignant gliomas, and diseases or disorders of other tissues such as acute respiratory distress syndrome, dry eye syndrome, and allergic conjunctivitis.

### BACKGROUND OF THE INVENTION

The restoration of body homeostasis after injuries or pathogen infections is critical to ensure the survival of an organism. The physiological wound healing and innate immune responses are initiated by the release of soluble mediators from the invading pathogens or injured lesions. The temporally regulated interactive repairing processes involve, for example, many chemokines, cytokines, acute phase proteins, infiltrating and tissue resident cells, fibroblasts, nerve cells, and vasculature. If the injury persists or is of an extensive magnitude, the physiological wound repair or anti-infection responses can become pathological, leading to excessive inflammation, edema, exuberant fibrosis and scarring, organ dysfunction, acute respiratory distress syndrome (ARDS), sepsis, and ultimately organ failure and/or death. Therefore, effective regulation of the magnitude and the duration of the inflammation and resolution responses is critical to organismal homeostasis. After tissue injury or pathogen infection (by bacteria, virus, fungus, and/or microbes), a set of formyl-peptides, damage-associated molecular pattern molecules (DAMPs), inflammatory lipid mediators (such as leukotrienes and lipoxins), and acute phase proteins (such as annexins) are released from invading pathogens, injured cells, and tissue lesions. Three formyl peptide receptors (FPR1, FPR2, and FPR3) serve as the key sensors for these chemotactic and activating molecules in humans. FPRs are highly expressed on neutrophils, macrophages, T lymphocytes, dendritic cells, epithelial cells, fibroblasts, microglia, and astrocytes. The binding of chemo-active molecules and acute proteins to the FPRs recruits leukocytes, stimulates superoxide and cytokine production, activates microglia and astrocytes, and elicits other inflammatory and resolution responses critical for injury repair and host defense.

On the other hand, disproportionate FPR receptor-mediated signaling can lead to pathological inflammatory responses and cause multiple diseases after injury or infection, including, for example, brain edema, functional impairment, and organ failure after stroke or traumatic brain injury (TBI). In addition, chronic activation of FPR-mediated signaling triggered by pathogens, tissue stress, and tissue injury has been implicated in the pathogenesis of brain cancer, gastric cancer, and Parkinson's disease.

Stroke is a leading cause of death globally with limited treatment options. The FPRs are highly expressed in microglia, astrocytes, and brain vasculature. After the onset of intracerebral hemorrhage (ICH), dying brain cells, activated platelets, microglia, and astrocytes release a spectrum of pro-inflammatory mediators, acute phase proteins, and DAMPs, which in turn activate FPR1. FPR1 activation induces leukocyte infiltration, reactive oxygen species (ROS) production, and cytokine release, which constitute the initial wave of inflammatory responses following ICH, contributing to the development of perihematomal edema and the aggravated mass effect in stroke.

TBI is a leading cause of disability worldwide. The global incidence rate of TBI is estimated at 200 per 100,000 people per year. Severe TBI frequently leads to behavioral disabilities, cerebral atrophy, dementia, permanent brain damage, and ultimately death. TBI has limited treatment options and FPR1 activation is involved in mediating the initial inflammatory processes of TBI.

Glioblastomas and malignant gliomas are the most common primary brain tumors. With an annual incidence of about 6 per 100,000 population. Malignant glioma has no effective treatment at present. FPR1 is highly expressed in glial cells, astrocytes, and brain vasculature; its interactions with the chemotactic ligands stemming from injury, stress, and pathogens are implicated in the pathophysiology of the brain cancers.

ARDS is a life-threatening lung injury that causes fluid leakage into the lungs and poor blood oxygenation. With an annual incidence of about 70 per 100,000 population, ARDS commonly occurs in patients hospitalized due to prior infection or trauma, has few treatment options, and has over 40% mortality rate for severe cases. Mitochondrial formylated peptides are elevated in lung fluids and serum of ARDS patients, and activation of FPR1 signaling is implicated as a key driver of acute lung injury.

Allergic conjunctivitis (AC) and dry eye syndrome (DES) are two of the most common inflammatory disorders of the eye, with estimated incidence rates up to 40% of the adult population. Current treatment options for AC and DES only partially alleviate the symptoms, and these disorders continue to have a strong negative effect on patients' quality of life. FPRs are expressed in the conjunctiva—the mucosal tissue layers of the eye, and their expression is elevated in the setting of inflammation. Thus, activation of FPR signaling is a potential mediator of the pathogenesis of AC and DES.

In view of the foregoing, there remains a need for new therapeutic agents and alternative mechanisms that can effectively address the limited treatment options currently available for stroke, TBI, glioblastomas, gliomas, ARDS, AC, and DES.

### SUMMARY OF THE INVENTION

One aspect of the present disclosure provides a compound selected from compounds of Formulae I, IIA, IIB, IIIA, IIIB, IVA, IVB, VA, VB, VIA, and VIB, a tautomer thereof, a deuterated derivative of the compound or the tautomer, or a pharmaceutically acceptable salt of the foregoing, which can be employed in the treatment of diseases mediated by the signaling of formyl peptide receptor 1 (FPR1). For example, disclosed herein is a compound of the following structural Formula I:

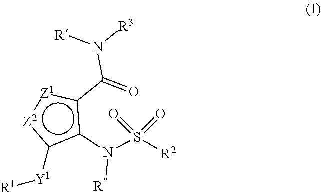

a tautomer thereof, a deuterated derivative of the compound or the tautomer, or a pharmaceutically acceptable salt the foregoing, wherein:
(i) each Z¹ and Z² is independently chosen from O, S, N, NR⁴, C(R⁴)₂, and CR⁴, and at least one of Z¹ and Z² is O, S, N, or NR⁴;
   wherein R⁴ is chosen from hydrogen, linear, branched, and cyclic alkyl groups, heterocyclic groups, aryl groups, and heteroaryl groups;
(ii) Y¹ is absent or chosen from a bond, O, S, or NR⁵; wherein R⁵ is chosen from hydrogen, linear, branched, and cyclic alkyl groups, heterocyclic groups, aryl groups, and heteroaryl groups;
(iii) R¹ is chosen from linear, branched, and cyclic alkyl groups, alkenyl groups, heterocyclic groups, aryl groups, and heteroaryl groups; or R¹ and Z², together with the atoms to which they are attached, form a cycloalkyl group, a heterocyclic group, an aryl group, or a heteroaryl group;
(iv) each R² and R³ is independently chosen from linear, branched, and cyclic alkyl groups, heterocyclic groups, aryl groups, and heteroaryl groups;
(v) each R′ and R″ is independently chosen from hydrogen, linear, branched, and cyclic alkyl groups, heterocyclic groups, aryl groups, and heteroaryl groups;
wherein the linear, branched, and cyclic alkyl groups, heterocyclic groups, aryl groups, and heteroaryl groups are optionally substituted with at least one group chosen from the following groups:
   halogen groups,
   hydroxy,
   thiol,
   amino,
   cyano,
   C₁-C₆ linear, branched, and cyclic alkyl groups,
   C₂-C₆ linear, branched, and cyclic alkenyl groups,
   C₁-C₆ linear, branched, and cyclic hydroxyalkyl groups,
   C₁-C₆ linear, branched, and cyclic alkoxy groups,
   C₁-C₆ linear, branched, and cyclic thioalkyl groups,
   C₁-C₆ linear, branched, and cyclic haloalkyl groups,
   C₁-C₆ linear, branched, and cyclic haloaminoalkyl groups,
   C₁-C₆ linear, branched, and cyclic halothioalkyl groups,
   C₁-C₆ linear, branched, and cyclic haloalkoxy groups,
   benzyloxy, benzylamino, and benzylthio groups,
   3 to 6-membered heterocycloalkenyl groups,
   3 to 6-membered heterocyclic groups, and
   5 and 6-membered heteroaryl groups.

In one aspect of the present disclosure, the compounds of Formula I are selected from Compounds 1 to 44 shown below, a tautomer thereof, a deuterated derivative of the compound or the tautomer, or a pharmaceutically acceptable salt of the foregoing.

In some embodiments, the present disclosure provides pharmaceutical compositions comprising a compound of Formulae I, IIA, IIB, IIIA, IIIB, IVA, IVB, VA, VB, VIA, and VIB, a tautomer thereof, a deuterated derivative of the compound or the tautomer, or a pharmaceutically acceptable salt of the foregoing, and a pharmaceutically acceptable carrier. In some embodiments, the pharmaceutical compositions may comprise a compound selected from Compounds 1 to 44 shown below, a tautomer thereof, a deuterated derivative of the compound or the tautomer, or a pharmaceutically acceptable salt of the foregoing. These compositions may further comprise an additional active pharmaceutical agent.

Another aspect of the present disclosure provides methods of treating a disease, a disorder, or a condition mediated by the signaling of formyl peptide receptor 1 (FPR1) in a subject, comprising administering a therapeutically effective amount of a compound of Formulae I, IIA, IIB, IIIA, IIIB, IVA, IVB, VA, VB, VIA, and VIB, a tautomer thereof, a deuterated derivative of the compound or the tautomer, or a pharmaceutically acceptable salt of the foregoing, or a pharmaceutical composition comprising any of the foregoing. In some embodiments, the methods of treatment comprise administering to a subject, a compound selected from Compounds 1 to 44 shown below, a tautomer thereof, a deuterated derivative of the compound or the tautomer, or a pharmaceutically acceptable salt of the foregoing, or a pharmaceutical composition comprising any of the foregoing.

In some embodiments disclosed herein, the methods of treatment comprise administration of an additional active pharmaceutical agent to the subject in need thereof, either in the same pharmaceutical composition as a compound of Formulae I, IIA, IIB, IIIA, IIIB, IVA, IVB, VA, VB, VIA, and VIB, a tautomer thereof, a deuterated derivative of the compound or the tautomer, or a pharmaceutically acceptable salt of the foregoing, or in a separate composition. In some embodiments disclosed herein, the methods of treatment comprise administering a compound selected from Compounds 1 to 44 shown below, a tautomer thereof, a deuterated derivative of the compound or the tautomer, or a pharmaceutically acceptable salt of the foregoing with an additional active pharmaceutical agent either in the same composition or in a separate composition.

Also disclosed herein are methods of modulating FPR1 activities, comprising administering to a subject a therapeutically effective amount of a compound of Formulae I, IIA, IIB, IIIA, IIIB, IVA, IVB, VA, VB, VIA, and VIB, a tautomer thereof, a deuterated derivative of the compound or the tautomer, or a pharmaceutically acceptable salt of the foregoing, or a pharmaceutical composition comprising any of the foregoing. In some embodiments disclosed herein, the methods of modulating FPR1 comprise administering to a subject, a compound selected from Compounds 1 to 44 shown below, a tautomer thereof, a deuterated derivative of the compound or the tautomer, or a pharmaceutically acceptable salt of the foregoing, or a pharmaceutical composition comprising any of the foregoing. In some embodiments, the methods of modulating FPR1 activity comprise contacting said FPR1 with a compound of Formulae I, IIA, IIB, IIIA, IIIB, IVA, IVB, VA, VB, VIA, and VIB, a tautomer thereof, a deuterated derivative of the compound or the tautomer, or a pharmaceutically acceptable salt of the foregoing, or a pharmaceutical composition comprising any of the foregoing. In some embodiments disclosed herein, the methods of modulating FPR1 comprise contacting the FPR1 with a compound selected from Compounds 1 to 44 shown below, a tautomer thereof, a deuterated derivative of the compound or the tautomer, or a pharmaceutically acceptable salt of the foregoing, or a pharmaceutical composition comprising any of the foregoing.

### DETAILED DESCRIPTION

### I. Definitions

The term “a” or “an” when referring to a noun as used herein encompasses the expression “at least one” and therefore encompasses both singular and plural units of the noun. For example, “an additional pharmaceutical agent” means a single or two or more additional pharmaceutical agents.

The term “FPR1” or “formyl peptide receptor 1” as used herein means the cell surface receptor protein that is encoded by the FPR1 gene in humans. FPR1 regulates a wide variety of neutrophil functional responses and plays an important role in the pathogenesis of various diseases, including, for example, the diseases set forth above.

The term “FPR1 modulator” as used herein refers to an organic chemistry small molecule compound (≤10 kDa) that has the ability to alter any one or more immune responses or signaling events mediated by FPR1, and can be either an FPR1 agonist or an FPR1 antagonist. If an FPR1 modulator is an agonist, the compound has the ability to increase any one or more immune responses or signaling events mediated by FPR1 from their native state, for example, by binding to the receptor to activate the receptor. If an FPR1 modulator is an antagonist, the compound has the ability to reduce or inhibit any one or more immune responses or signaling events mediated by FPR1 from their native state, for example, by blocking the agonist binding site or an allosteric binding site on the receptor in order to achieve the reduced or inhibited effects.

The term “compound,” when referring to a compound of the present disclosure, refers to a collection of molecules having an identical chemical structure unless otherwise indicated as a collection of stereoisomers (for example, a collection of racemates, a collection of cis/trans stereoisomers, or a collection of (E) and (Z) stereoisomers), except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of the present disclosure will depend upon a number of factors, including, for example, the isotopic purity of reagents used to make the compound and the efficiency of incorporation of isotopes in the various synthesis steps used to prepare the compound. However, as set forth above the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

As used herein, “optionally substituted” is interchangeable with the phrase “substituted or unsubstituted.” In general, the term “substituted,” refers to the replacement of hydrogen radicals in a given structure with the radical of a specified substituent. Unless otherwise indicated, an “optionally substituted” group may have a substituent at each substitutable position of the group, and when more than one position in any given structure may be substituted with more than one substituent chosen from a specified group, the substituent may be either the same or different at every position. Combinations of substituents envisioned by the present disclosure are those that result in the formation of stable or chemically feasible compounds.

The term “isotopologue” refers to a species in which the chemical structure differs from only in the isotopic composition thereof. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C or ¹⁴C are within the scope of the present disclosure.

Unless otherwise indicated, structures depicted herein are also meant to include all isomeric forms of the structure, e.g., racemic mixtures, cis/trans isomers, geometric (or conformational) isomers, such as (Z) and (E) double bond isomers, and (Z) and (E) conformational isomers. Therefore, geometric and conformational mixtures of the present compounds are within the scope of the present disclosure. Unless otherwise stated, all tautomeric forms of the compounds of the present disclosure are within the scope of the present disclosure.

The term “tautomer,” as used herein, refers to one of two or more isomers of compound that exist together in equilibrium, and are readily interchanged by migration of an atom, e.g., a hydrogen atom, or group within the molecule.

“Stereoisomer” as used herein refers to enantiomers and diastereomers.

As used herein, “deuterated derivative” refers to a compound having the same chemical structure as a reference compound, but with one or more hydrogen atoms replaced by a deuterium atom (“D” or “²H”). It will be recognized that some variation of natural isotopic abundance occurs in a synthesized compound depending on the origin of chemical materials used in the synthesis. The concentration of naturally abundant stable hydrogen isotopes, notwithstanding this variation is small and immaterial as compared to the degree of stable isotopic substitution of deuterated derivatives described herein. Thus, unless otherwise stated, when a reference is made to a “deuterated derivative” of a compound of the present disclosure, at least one hydrogen is replaced with deuterium at a level that is well above its natural isotopic abundance, which is typically about 0.015%. In some embodiments, the deuterated derivatives disclosed herein have an isotopic enrichment factor for each deuterium atom, of at least 3500 (52.5% deuterium incorporation at each designated deuterium), at least 4500 (67.5% deuterium incorporation at each designated deuterium), at least 5000 (75% deuterium incorporation at each designated deuterium), at least 5500 (82.5% deuterium incorporation at each designated deuterium), at least 6000 (90% deuterium incorporation at each designated deuterium), at least 6333.3 (95% deuterium incorporation at each designated deuterium), at least 6466.7 (97% deuterium incorporation at each designated deuterium), or at least 6600 (99% deuterium incorporation at each designated deuterium).

The term “isotopic enrichment factor” as used herein means the ratio between the isotopic abundance and the natural abundance of a specified isotope.

The term “alkyl” as used herein, means a linear or branched, substituted or unsubstituted hydrocarbon chain that is completely saturated. Unless otherwise specified, an alkyl group contains 1 to 30 alkyl carbon atoms. In some embodiments, an alkyl group contains 1 to 20 alkyl carbon atoms. In some embodiments, an alkyl group contains 1 to 10 aliphatic carbon atoms. In some embodiments, an alkyl group contains 1 to 8 aliphatic carbon atoms. In some embodiments, an alkyl group contains 1 to 6 alkyl carbon atoms. In some embodiments, an alkyl group contains 1 to 4 alkyl carbon atoms. In other embodiments, an alkyl group contains 1 to 3 alkyl carbon atoms. And in yet other embodiments, an alkyl group contains 1 to 2 alkyl carbon atoms. In some embodiments, alkyl groups are substituted. In some embodiments, alkyl groups are unsubstituted. In some embodiments, alkyl groups are linear or straight-chain or unbranched. In some embodiments, alkyl groups are branched.

The term “cycloalkyl” refers to a monocyclic C₃₋₈ hydrocarbon or a spirocyclic, fused, or bridged bicyclic or tricyclic C₈₋₁₄ hydrocarbon that is completely saturated, wherein any individual ring in said bicyclic ring system has 3 to 7 members. In some embodiments, cycloalkyl groups are substituted. In some embodiments, cycloalkyl groups are unsubstituted. In some embodiments, the cycloalkyl is a C₃ to C₁₂ cycloalkyl. In some embodiments, the cycloalkyl is a C₃ to C₈ cycloalkyl. In some embodiments, the cycloalkyl is a C₃ to C₆ cycloalkyl. Non-limiting examples of monocyclic cycloalkyls include cyclopropyl, cyclobutyl, cyclopentanyl, and cyclohexyl.

The term “carbocyclyl” encompasses the term “cycloalkyl” and refers to a monocyclic C₃₋₈ hydrocarbon or a spirocyclic, fused, or bridged bicyclic or tricyclic C₈₋₁₄ hydrocarbon that is completely saturated, or is partially saturated as it contains one or more units of unsaturation but is not aromatic, wherein any individual ring in said bicyclic ring system has 3 to 7 members. Bicyclic carbocyclyls include combinations of a monocyclic carbocyclic ring fused to, for example, a phenyl. In some embodiments, carbocyclyl groups are substituted. In some embodiments, carbocyclyl groups are unsubstituted. In some embodiments, the carbocyclyl is a C₃ to C₁₂ carbocyclyl. In some embodiments, the carbocyclyl is a C₃ to C₁₀ carbocyclyl. In some embodiments, the carbocyclyl is a C₃ to C₈ carbocyclyl. Non-limiting examples of monocyclic carbocyclyls include cyclopropyl, cyclobutyl, cyclopentanyl, cyclohexyl, and cyclopentenyl, cyclohexenyl.

The term “alkenyl” as used herein, means a linear or branched, substituted or unsubstituted hydrocarbon chain that contains one or more double bonds. In some embodiments, alkenyl groups are substituted. In some embodiments, alkenyl groups are unsubstituted. In some embodiments, alkenyl groups are linear, straight-chain, or unbranched. In some embodiments, alkenyl groups are branched. In some embodiments, alkenyl groups are cyclic. In some embodiments, the alkenyl group is a C₃ to C₁₂ alkenyl group. In some embodiments, the alkenyl group is a C₃ to C₈ alkenyl group. In some embodiments, the alkenyl group is a C₃ to C₆ alkenyl group. Non-limiting examples of alkenyl group include allyl, butenyl, pentenyl, cyclopentenyl, hexenyl, cyclohexenyl, and heptenyl.

The term “heterocyclyl” as used herein means non-aromatic (i.e., completely saturated or partially saturated as in it contains one or more units of unsaturation but is not aromatic), monocyclic, or spirocyclic, fused, or bridged bicyclic or tricyclic ring systems in which one or more ring members is an independently chosen heteroatom. Bicyclic heterocyclyls include, for example, the following combinations of monocyclic rings: a monocyclic heteroaryl fused to a monocyclic heterocyclyl; a monocyclic heterocyclyl fused to another monocyclic heterocyclyl; a monocyclic heterocyclyl fused to phenyl; a monocyclic heterocyclyl fused to a monocyclic carbocyclyl/cycloalkyl; and a monocyclic heteroaryl fused to a monocyclic carbocyclyl/cycloalkyl. In some embodiments, the “heterocyclyl” group contains 3 to 14 ring members in which one or more ring members is a heteroatom independently chosen, for example, from oxygen, sulfur, nitrogen, and phosphorus. In some embodiments, each ring in a bicyclic or tricyclic ring system contains 3 to 7 ring members. In some embodiments, the heterocycle has at least one unsaturated carbon-carbon bond. In some embodiments, the heterocycle has at least one unsaturated carbon-nitrogen bond. In some embodiments, the heterocycle has one heteroatom independently chosen from oxygen, sulfur, nitrogen, and phosphorus. In some embodiments, the heterocycle has one heteroatom that is a nitrogen atom. In some embodiments, the heterocycle has one heteroatom that is an oxygen atom. In some embodiments, the heterocycle has two heteroatoms that are each independently selected from nitrogen and oxygen. In some embodiments, the heterocycle has three heteroatoms that are each independently selected from nitrogen and oxygen. In some embodiments, heterocycles are substituted. In some embodiments, heterocycles are unsubstituted. In some embodiments, the heterocyclyl is a 3- to 12-membered heterocyclyl. In some embodiments, the heterocyclyl is a 4- to 10-membered heterocyclyl. In some embodiments, the heterocyclyl is a 3- to 8-membered heterocyclyl. In some embodiments, the heterocyclyl is a 5- to 10-membered heterocyclyl. In some embodiments, the heterocyclyl is a 5- to 8-membered heterocyclyl. In some embodiments, the heterocyclyl is a 5- or 6-membered heterocyclyl. In some embodiments, the heterocyclyl is a 6-membered heterocyclyl. Non-limiting examples of monocyclic heterocyclyls include piperidinyl, piperazinyl, morpholinyl, tetrahydropyranyl, azetidinyl, oxetanyl, tetrahydrothiophenyl, dihyropyranyl, and tetrahydropyridinyl.

The term “heteroatom” means one or more of oxygen, sulfur, and nitrogen, including, any oxidized form of nitrogen or sulfur, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl).

The term “unsaturated”, as used herein, means that a moiety has one or more units or degrees of unsaturation. Unsaturation is the state in which not all of the available valence bonds in a compound are satisfied by substituents and thus the compound contains double or triple bonds.

The term “alkoxy” as used herein, refers to an alkyl group, as defined above, wherein one carbon of the alkyl group is replaced by an oxygen (“alkoxy”) atom, provided that the oxygen atom is linked between two carbon atoms.

The term “halogen” includes F, Cl, Br, and I, i.e., fluoro, chloro, bromo, and iodo, respectively.

As used herein, a “cyano” or “nitrile” group refer to —C≡N.

As used herein, an “aromatic ring” refers to a carbocyclic or heterocyclic ring that contains conjugated, planar ring systems with delocalized pi electron orbitals comprised of [4n+2] p orbital electrons, wherein n is an integer of 0 to 6. A “non-aromatic” ring refers to a carbocyclic or heterocyclic that does not meet the requirements set forth above for an aromatic ring, and can be either completely or partially saturated. Nonlimiting examples of aromatic rings include aryl and heteroaryl rings that are further defined as follows.

The term “aryl” used alone or as part of a larger moiety as in “arylalkyl,” “arylalkoxy,” or “aryloxyalkyl,” refers to monocyclic or spirocyclic, fused, or bridged bicyclic or tricyclic ring systems having a total of five to fourteen ring members, wherein every ring in the system is an aromatic ring containing only carbon atoms and wherein each ring in a bicyclic or tricyclic ring system contains 3 to 7 ring members. Nonlimiting examples of aryl groups include phenyl (C₆) and naphthyl (C₁₀) rings. In some embodiments, aryl groups are substituted. In some embodiments, aryl groups are unsubstituted.

The term “heteroaryl” refers to monocyclic or spirocyclic, fused, or bridged bicyclic or tricyclic ring systems having a total of five to fourteen ring members, wherein at least one ring in the system is aromatic, at least one ring in the system contains one or more heteroatoms, and wherein each ring in a bicyclic or tricyclic ring system contains 3 to 7 ring members. Bicyclic heteroaryls include, for example, the following combinations of monocyclic rings: a monocyclic heteroaryl fused to another monocyclic heteroaryl; and a monocyclic heteroaryl fused to a phenyl. In some embodiments, heteroaryl groups are substituted. In some embodiments, heteroaryl groups have one or more heteroatoms chosen, for example, from nitrogen, oxygen, and sulfur. In some embodiments, heteroaryl groups have one heteroatom. In some embodiments, heteroaryl groups have two heteroatoms. In some embodiments, heteroaryl groups are monocyclic ring systems having five ring members. In some embodiments, heteroaryl groups are monocyclic ring systems having six ring members. In some embodiments, heteroaryl groups are unsubstituted. In some embodiments, the heteroaryl is a 3- to 12-membered heteroaryl. In some embodiments, the heteroaryl is a 3- to 10-membered heteroaryl. In some embodiments, the heteroaryl is a 3- to 8-membered heteroaryl. In some embodiments, the heteroaryl is a 5- to 10-membered heteroaryl. In some embodiments, the heteroaryl is a 5- to 8-membered heteroaryl. In some embodiments, the heteroaryl is a 5- or 6-membered heteroaryl. Non-limiting examples of monocyclic heteroaryls are pyridinyl, pyrimidinyl, thiophenyl, thiazolyl, and isoxazolyl.

A “spirocyclic ring system” refers to a ring system having two or more cyclic rings, where every two rings share only one common atom.

Non-limiting examples of suitable solvents that may be used in the present disclosure include water, methanol (MeOH), ethanol (EtOH), dichloromethane or “methylene chloride” (CH₂Cl₂), toluene, acetonitrile (MeCN), dimethylformamide (DMF), dimethyl sulfoxide (DMSO), methyl acetate (MeOAc), ethyl acetate (EtOAc), heptanes, isopropyl acetate (IPAc), tert-butyl acetate (t-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1,4-dioxane, and N-methyl pyrrolidone (NMP).

Non-limiting examples of suitable bases that may be used in the present disclosure include 1,8-diazabicyclo[5.4.0]undec-7-ene (DBU), potassium tert-butoxide (KOtBu), potassium carbonate (K₂CO₃), N-methylmorpholine (NMM), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH) and sodium methoxide (NaOMe; NaOCH₃).

Disclosed herein are pharmaceutically acceptable salts of the disclosed compounds. A salt of a compound is formed between an acid and a basic group of the compound, such as an amino functional group, or a base and an acidic group of the compound, such as a carboxyl functional group.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of the present disclosure. Suitable pharmaceutically acceptable salts are, for example, those disclosed in S. M. Berge, et al. J. Pharmaceutical Sciences, 1977, 66, pp. 1 to 19.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In some embodiments, pharmaceutically acceptable acid addition salts include those formed with mineral acids such as hydrochloric acid and hydrobromic acid, and those formed with organic acids such as maleic acid.

Pharmaceutically acceptable salts derived from appropriate bases include alkali metal, alkaline earth metal, ammonium, and N⁺(C₁₋₄alkyl)₄ salts. The present disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

The term “subject” refers to an animal, including but not limited to a human.

The term “therapeutically effective amount” refers to that amount of a compound that produces the desired effect for which it is administered (e.g., improvement in symptoms of diseases, disorders, and conditions mediated by the signaling of FPR1, lessening the severity of diseases, disorders, and conditions mediated by the signaling of FPR1 or a symptom thereof, and/or reducing progression of diseases, disorders, and conditions mediated by the signaling of FPR1 or a symptom thereof). The exact amount of a therapeutically effective amount will depend on the purpose of the treatment and will be ascertainable by one skilled in the art using known techniques (see, e.g., Lloyd (1999), The Art, Science and Technology of Pharmaceutical Compounding).

As used herein, the term “treatment” and its cognates refer to slowing or stopping disease progression. “Treatment” and its cognates as used herein include, but are not limited to the following: complete or partial remission, lower risk of diseases, disorders, and conditions mediated by FPR1 signaling, and disease-related complications. Improvements in or lessening the severity of any of these symptoms can be readily assessed according to methods and techniques known in the art or subsequently developed.

The terms “about” and “approximately,” when used in connection with doses, amounts, or weight percent of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent.

### II. Compounds and Compositions

In a first embodiment, a compound of the present disclosure is a compound of the following structural formula I:

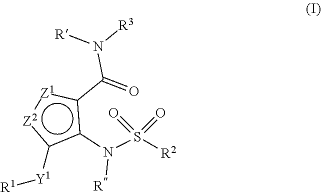

a tautomer thereof, a deuterated derivative of the compound or the tautomer, or a pharmaceutically acceptable salt the foregoing, wherein:
(i) each Z¹ and Z² is independently chosen from O, S, N, NR⁴, C(R⁴)₂, and CR⁴, and at least one of Z¹ and Z² is O, S, N, or NR⁴;
wherein R⁴ is chosen from hydrogen, linear, branched, and cyclic alkyl groups, carbocyclic groups, heterocyclic groups, aryl groups, and heteroaryl groups;
(ii) Y¹ is absent or is chosen from O, S, and NR⁵;
wherein R⁵ is chosen from hydrogen, linear, branched, and cyclic alkyl groups, heterocyclic groups, aryl groups, and heteroaryl groups;
(iii) R¹ is chosen from linear, branched, and cyclic alkyl groups, alkenyl groups, carbocyclic groups, heterocyclic groups, linear, branched, and cyclic alkenyl groups, linear and branched heteroalkenyl groups, aryl groups, and heteroaryl groups; or R¹ and Z², together with the atoms to which they are attached, form a cycloalkyl group, a heterocyclic group, an aryl group, or a heteroaryl group;
(iv) each R² and R³ is independently chosen from linear, branched, and cyclic alkyl groups, carbocyclic groups, heterocyclic groups, aryl groups, and heteroaryl groups;
(v) each R′ and R″ is independently chosen from hydrogen, linear, branched, and cyclic alkyl groups, carbocyclic groups, heterocyclic groups, aryl groups, and heteroaryl groups;
wherein the linear, branched, and cyclic alkyl groups, linear, branched, and cyclic alkenyl groups, linear and branched heteroalkenyl groups, carbocyclic groups, heterocyclic groups, aryl groups, and heteroaryl groups are optionally substituted with at least one group chosen from the following groups:
   halogen groups,
   hydroxy,
   thiol,
   amino,
   cyano,
   —OC(O)C₁-C₆ linear, branched, and cyclic alkyl groups,
   —C(O)OC₁-C₆ linear, branched, and cyclic alkyl groups,
   —NHC₁-C₆ linear, branched, and cyclic alkyl groups,
   —N(C₁-C₆ linear, branched, and cyclic alkyl groups)₂,
   —NHC(O)C₁-C₆ linear, branched, and cyclic alkyl groups,
   —C(O)NHC₁-C₆ linear, branched, and cyclic alkyl groups,
   —NHaryl groups,
   —N(aryl groups)₂,
   —NHC(O)aryl groups,
   —C(O)NHaryl groups,
   —NHheteroaryl groups,
   —N(heteroaryl groups)₂,
   —NHC(O)heteroaryl groups,
   —C(O)NHheteroaryl groups,
   C₁-C₆ linear, branched, and cyclic alkyl groups,
   C₂-C₆ linear, branched, and cyclic alkenyl groups,
   C₁-C₆ linear, branched, and cyclic hydroxyalkyl groups,
   C₁-C₆ linear, branched, and cyclic alkoxy groups,
   C₁-C₆ linear, branched, and cyclic thioalkyl groups,
   C₁-C₆ linear, branched, and cyclic haloalkyl groups,
   C₁-C₆ linear, branched, and cyclic haloaminoalkyl groups,
   C₁-C₆ linear, branched, and cyclic halothioalkyl groups,
   C₁-C₆ linear, branched, and cyclic haloalkoxy groups,
   benzyloxy, benzylamino, and benzylthio groups,
   3 to 6-membered heterocycloalkenyl groups,
   3 to 6-membered heterocyclic groups, and
   5 and 6-membered heteroaryl groups.

In a second embodiment, a compound of the present disclosure is of one of the following structural formula IIA or IIB:

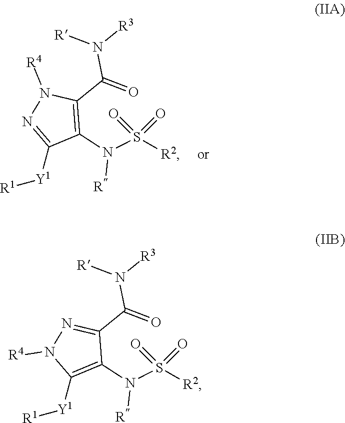

a tautomer thereof, a deuterated derivative of the compound or the tautomer, or a pharmaceutically acceptable salt of the foregoing; and all other variables not specifically defined herein are as defined in the first embodiment.

In a third embodiment, a compound of the present disclosure is of one of the following structural formula IIIA or IIIB:

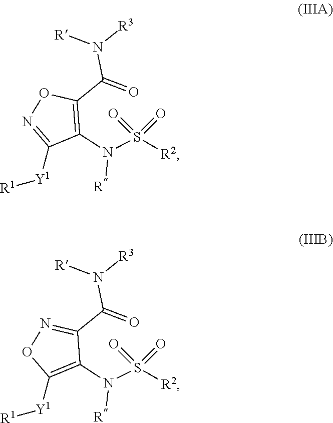

a tautomer thereof, a deuterated derivative of the compound or the tautomer, or a pharmaceutically acceptable salt of the foregoing; and all other variables not specifically defined herein are as defined in the first embodiment.

In a fourth embodiment, a compound of the present disclosure is of one of the following structural formula IVA or IVB:

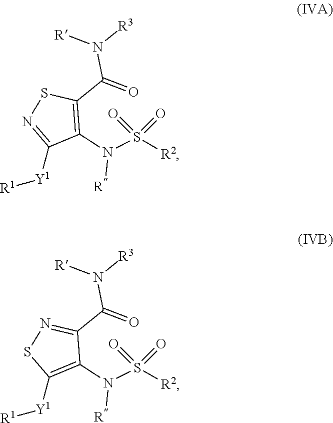

a tautomer thereof, a deuterated derivative of the compound or the tautomer, or a pharmaceutically acceptable salt of the foregoing; and all other variables not specifically defined herein are as defined in the first embodiment.

In a fifth embodiment, a compound of the present disclosure is of one of the following structural formula VA or VB:

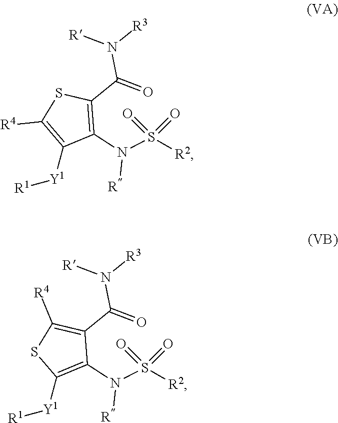

a tautomer thereof, a deuterated derivative of the compound or the tautomer, or a pharmaceutically acceptable salt of the foregoing; and all other variables not specifically defined herein are as defined in the first embodiment.

In a sixth embodiment, a compound of the present disclosure is of one of the following structural formula VIA or VIB:

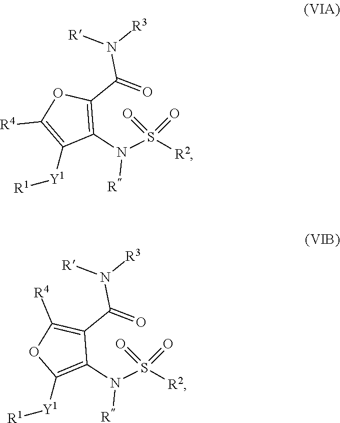

a tautomer thereof, a deuterated derivative of the compound or the tautomer, or a pharmaceutically acceptable salt of the foregoing; and all other variables not specifically defined herein are as defined in the first embodiment.

In a seventh embodiment, in a compound, tautomer, deuterated derivative, or pharmaceutically acceptable salt of the present disclosure, R¹ is chosen from cyclic alkyl groups, heterocyclic groups, aryl groups, and heteroaryl groups; R² is an aryl group; and R³ is chosen from linear and branched alkyl groups; and all other variables not specifically defined herein are as defined in any one of the first, second, third, fourth, fifth, or sixth embodiments.

In an eighth embodiment, in a compound, tautomer, deuterated derivative, or pharmaceutically acceptable salt of the present disclosure, R¹ is chosen from optionally substituted 5 and 6-membered aryl groups; and all other variables not specifically defined herein are as defined in any one of the first, second, third, fourth, fifth, or sixth embodiments.

In a ninth embodiment, in a compound, tautomer, deuterated derivative, or pharmaceutically acceptable salt of the present disclosure, R¹ is chosen from 5 and 6-membered aryl groups substituted with at least one halogen group; and all other variables not specifically defined herein are as defined in any one of the first, second, third, fourth, fifth, or sixth embodiments.

In a tenth embodiment, in a compound, tautomer, deuterated derivative, or pharmaceutically acceptable salt of the present disclosure, R¹ is chosen from 5 and 6-membered aryl groups substituted with at least one fluoro; and all other variables not specifically defined herein are as defined in any one of the first, second, third, fourth, fifth, or sixth embodiments.

In a eleventh embodiment, in a compound, tautomer, deuterated derivative, or pharmaceutically acceptable salt of the present disclosure, R¹ is chosen from 5 and 6-membered aryl groups substituted with at least one chloro; and all other variables not specifically defined herein are as defined in any one of the first, second, third, fourth, fifth, or sixth embodiments.

In a twelfth embodiment, in a compound, tautomer, deuterated derivative, or pharmaceutically acceptable salt of the present disclosure, R¹ is chosen from optionally substituted 5 and 6-membered heteroaryl groups; and all other variables not specifically defined herein are as defined in any one of the first, second, third, fourth, fifth, or sixth embodiments.

In a thirteenth embodiment, in a compound, tautomer, deuterated derivative, or pharmaceutically acceptable salt of the present disclosure, R¹ is chosen from 5 and 6-membered heteroaryl groups substituted with at least one halogen group; and all other variables not specifically defined herein are as defined in any one of the first, second, third, fourth, fifth, or sixth embodiments.

In a fourteenth embodiment, in a compound, tautomer, deuterated derivative, or pharmaceutically acceptable salt of the present disclosure, R¹ is chosen from 5 and 6-membered heteroaryl groups substituted with at least one fluoro; and all other variables not specifically defined herein are as defined in any one of the first, second, third, fourth, fifth, or sixth embodiments.

In a fifteenth embodiment, in a compound, tautomer, deuterated derivative, or pharmaceutically acceptable salt of the present disclosure, R¹ is chosen from 5 and 6-membered heteroaryl group substituted with at least one chloro; and all other variables not specifically defined herein are as defined in any one of the first, second, third, fourth, fifth, or sixth embodiments.

In a sixteenth embodiment, in a compound, tautomer, deuterated derivative, or pharmaceutically acceptable salt of the present disclosure, R¹ is chosen from optionally substituted 3 to 6-membered cyclic alkyl groups; and all other variables not specifically defined herein are as defined in any one of the first, second, third, fourth, fifth, or sixth embodiments.

In a seventeenth embodiment, in a compound, tautomer, deuterated derivative, or pharmaceutically acceptable salt of the present disclosure, R¹ is chosen from 3 to 6-membered cyclic alkyl groups substituted with at least one alkoxy group; and all other variables not specifically defined herein are as defined in any one of the first, second, third, fourth, fifth, or sixth embodiments.

In an eighteenth embodiment, in a compound, tautomer, deuterated derivative, or pharmaceutically acceptable salt of the present disclosure, R¹ is chosen from 3 to 6-membered cyclic alkyl groups substituted with at least one methoxy group; and all other variables not specifically defined herein are as defined in any one of the first, second, third, fourth, fifth, or sixth embodiments.

In a nineteenth embodiment, in a compound, tautomer, deuterated derivative, or pharmaceutically acceptable salt of the present disclosure, R¹ is chosen from optionally substituted 3 to 6-membered heterocyclic groups; and all other variables not specifically defined herein are as defined in any one of the first, second, third, fourth, fifth, or sixth embodiments.

In a twentieth embodiment, in a compound, tautomer, deuterated derivative, or pharmaceutically acceptable salt of the present disclosure, R² is chosen from optionally substituted 5 and 6-membered heteroaryl groups; and all other variables not specifically defined herein are as defined in any one of the first, second, third, fourth, fifth, or sixth embodiments.

In a twenty-first embodiment, in a compound, tautomer, deuterated derivative, or pharmaceutically acceptable salt of the present disclosure, R² is chosen from 5 and 6-membered heteroaryl groups substituted with at least one halogen group; and all other variables not specifically defined herein are as defined in any one of the first, second, third, fourth, fifth, or sixth embodiments.

In a twenty-second embodiment, in a compound, tautomer, deuterated derivative, or pharmaceutically acceptable salt of the present disclosure, R² is chosen from 5 and 6-membered heteroaryl groups substituted with at least one fluoro; and all other variables not specifically defined herein are as defined in any one of the first, second, third, fourth, fifth, or sixth embodiments.

In a twenty-third embodiment, in a compound, tautomer, deuterated derivative, or pharmaceutically acceptable salt of the present disclosure, R² is chosen from 5 and 6-membered heteroaryl groups substituted with at least one chloro; and all other variables not specifically defined herein are as defined in any one of the first, second, third, fourth, fifth, or sixth embodiments.

In a twenty-fourth embodiment, in a compound, tautomer, deuterated derivative, or pharmaceutically acceptable salt of the present disclosure, R² is chosen from 5 and 6-membered heteroaryl group substituted with at least one cyano; and all other variables not specifically defined herein are as defined in any one of the first, second, third, fourth, fifth, or sixth embodiments.

In a twenty-fifth embodiment, in a compound, tautomer, deuterated derivative, or pharmaceutically acceptable salt of the present disclosure, R² is chosen from 5 and 6-membered heteroaryl groups substituted with at least one group chosen from C₁-C₆ linear, C₃-C₆ branched, and C₃-C₆ cyclic alkyl groups; and all other variables not specifically defined herein are as defined in any one of the first, second, third, fourth, fifth, or sixth embodiments.

In a twenty-sixth embodiment, in a compound, tautomer, deuterated derivative, or pharmaceutically acceptable salt of the present disclosure, R³ is chosen from optionally substituted C₁-C₁₀ linear and C₂-C₁₀ branched alkyl groups; and all other variables not specifically defined herein are as defined in any one of the first, second, third, fourth, fifth, or sixth embodiments.

In a twenty-seventh embodiment, in a compound, tautomer, deuterated derivative, or pharmaceutically acceptable salt of the present disclosure, R³ is chosen from methyl, ethyl, propyl, isopropyl, butyl, iso-butyl, sec-butyl, pentyl, iso-pentyl, sec-pentyl, neo-pentyl, and 1,2,2-trimethylpropyl; and all other variables not specifically defined herein are as defined in any one of the first, second, third, fourth, fifth, or sixth embodiments.

In a twenty-eighth embodiment, in a compound, tautomer, deuterated derivative, or pharmaceutically acceptable salt of the present disclosure; and all other variables not specifically defined herein are as defined in any one of the first, second, third, fourth, fifth, or sixth embodiments.

In a twenty-ninth embodiment, in a compound, tautomer, deuterated derivative, or pharmaceutically acceptable salt of the present disclosure, R⁴ is chosen from optionally substituted C₁-C₁₀ linear, C₂-C₁₀ branched alkyl groups, and C₃-C₆ cyclic alkyl groups; and all other variables not specifically defined herein are as defined in any one of the first, second, third, fourth, fifth, or sixth embodiments.

In a thirtieth embodiment, in a compound, tautomer, deuterated derivative, or pharmaceutically acceptable salt of the present disclosure, R⁴ is chosen from methyl, ethyl, propyl, isopropyl, cyclopropyl, butyl, iso-butyl, sec-butyl, cyclobutyl, pentyl, iso-pentyl, sec-pentyl, neo-pentyl, 1,2,2-trimethylpropyl, cyclopentyl, and cyclohexyl; and all other variables not specifically defined herein are as defined in any one of the first, second, third, fourth, fifth, or sixth embodiments.

In a thirty-first embodiment, in a compound, tautomer, deuterated derivative, or pharmaceutically acceptable salt of the present disclosure, R′ is hydrogen; and all other variables not specifically defined herein are as defined in any one of the first, second, third, fourth, fifth, or sixth embodiments.

In a thirty-second embodiment, in a compound, tautomer, deuterated derivative, or pharmaceutically acceptable salt of the present disclosure, R″ is hydrogen; and all other variables not specifically defined herein are as defined in any one of the first, second, third, fourth, fifth, or sixth embodiments.

In certain embodiments, the at least one compound of the present disclosure is selected from Compounds 1 to 44 depicted in Table 1, a tautomer thereof, a deuterated derivative of the compound or the tautomer, or a pharmaceutically acceptable salt of the foregoing.

| | |
|---|---|
| 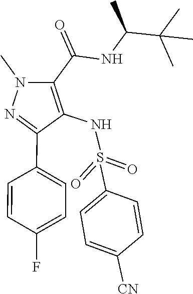 [SMILES: C[C@H](NC(=O)c1c(NS(=O)(=O)c2ccc(C#N)cc2)c(-c2ccc(F)cc2)nn1C)C(C)(C)C] | 1 |
| 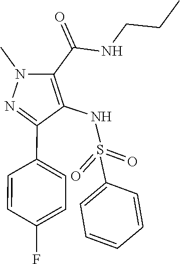 [SMILES: CCCNC(=O)c1c(NS(=O)(=O)c2ccccc2)c(-c2ccc(F)cc2)nn1C] | 2 |
| 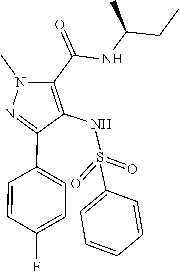 [SMILES: CC[C@H](C)NC(=O)c1c(NS(=O)(=O)c2ccccc2)c(-c2ccc(F)cc2)nn1C] | 3 |
| 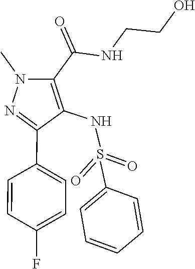 [SMILES: Cn1nc(-c2ccc(F)cc2)c(NS(=O)(=O)c2ccccc2)c1C(=O)NCCO] | 4 |
| 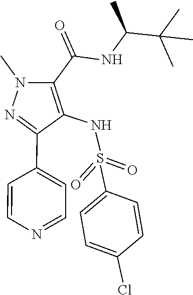 [SMILES: C[C@H](NC(=O)c1c(NS(=O)(=O)c2ccc(Cl)cc2)c(-c2ccncc2)nn1C)C(C)(C)C] | 5 |
| 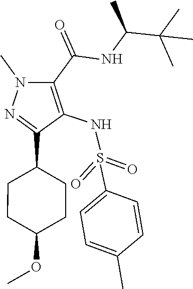 [SMILES: CO[C@H]1CC[C@@H](c2nn(C)c(C(=O)N[C@@H](C)C(C)(C)C)c2NS(=O)(=O)c2ccc(C)cc2)CC1] | 6 |
| 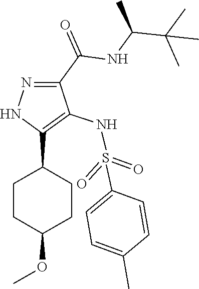 [SMILES: CO[C@H]1CC[C@@H](c2[nH]nc(C(=O)N[C@@H](C)C(C)(C)C)c2NS(=O)(=O)c2ccc(C)cc2)CC1] | 7 |
| 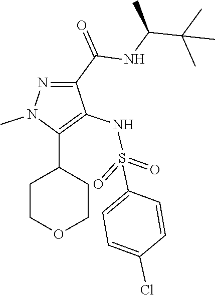 [SMILES: C[C@H](NC(=O)c1nn(C)c(C2CCOCC2)c1NS(=O)(=O)c1ccc(Cl)cc1)C(C)(C)C] | 8 |
| 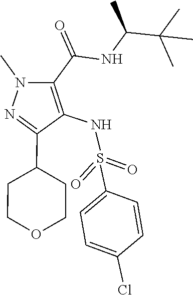 [SMILES: C[C@H](NC(=O)c1c(NS(=O)(=O)c2ccc(Cl)cc2)c(C2CCOCC2)nn1C)C(C)(C)C] | 9 |
| 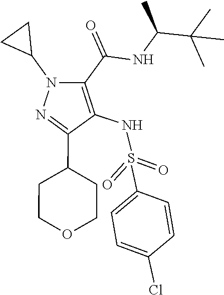 [SMILES: C[C@H](NC(=O)c1c(NS(=O)(=O)c2ccc(Cl)cc2)c(C2CCOCC2)nn1C1CC1)C(C)(C)C] | 10 |
| 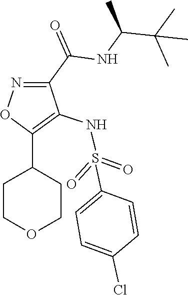 [SMILES: C[C@H](NC(=O)c1noc(C2CCOCC2)c1NS(=O)(=O)c1ccc(Cl)cc1)C(C)(C)C] | 11 |
| 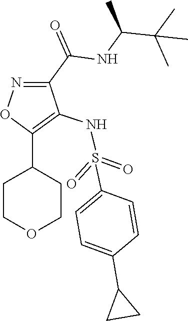 [SMILES: C[C@H](NC(=O)c1noc(C2CCOCC2)c1NS(=O)(=O)c1ccc(C2CC2)cc1)C(C)(C)C] | 12 |
| 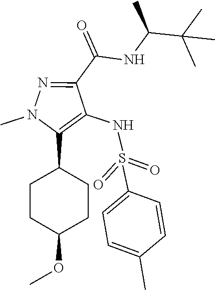 [SMILES: CO[C@H]1CC[C@@H](c2c(NS(=O)(=O)c3ccc(C)cc3)c(C(=O)N[C@@H](C)C(C)(C)C)nn2C)CC1] | 13 |
| 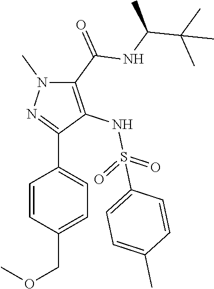 [SMILES: COCc1ccc(-c2nn(C)c(C(=O)N[C@@H](C)C(C)(C)C)c2NS(=O)(=O)c2ccc(C)cc2)cc1] | 14 |
| 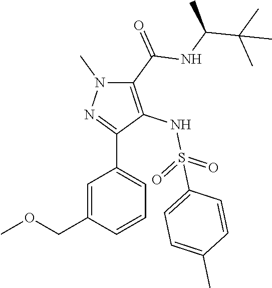 [SMILES: COCc1cccc(-c2nn(C)c(C(=O)N[C@@H](C)C(C)(C)C)c2NS(=O)(=O)c2ccc(C)cc2)c1] | 15 |
| 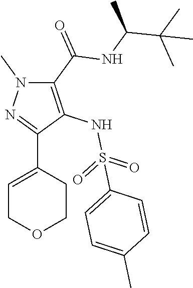 [SMILES: Cc1ccc(S(=O)(=O)Nc2c(C3=CCOCC3)nn(C)c2C(=O)N[C@@H](C)C(C)(C)C)cc1] | 16 |
| 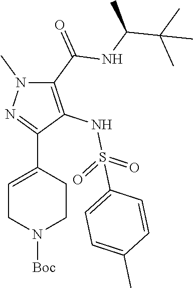 [SMILES: Cc1ccc(S(=O)(=O)Nc2c(C3=CCN(C(=O)OC(C)(C)C)CC3)nn(C)c2C(=O)N[C@@H](C)C(C)(C)C)cc1] | 17 |
| 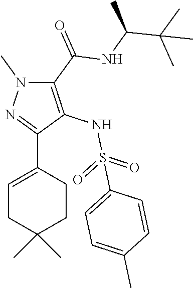 [SMILES: Cc1ccc(S(=O)(=O)Nc2c(C3=CCC(C)(C)CC3)nn(C)c2C(=O)N[C@@H](C)C(C)(C)C)cc1] | 18 |
| 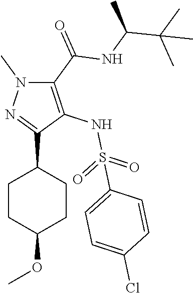 [SMILES: CO[C@H]1CC[C@@H](c2nn(C)c(C(=O)N[C@@H](C)C(C)(C)C)c2NS(=O)(=O)c2ccc(Cl)cc2)CC1] | 19 |
| 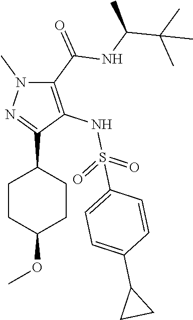 [SMILES: CO[C@H]1CC[C@@H](c2nn(C)c(C(=O)N[C@@H](C)C(C)(C)C)c2NS(=O)(=O)c2ccc(C3CC3)cc2)CC1] | 20 |
| 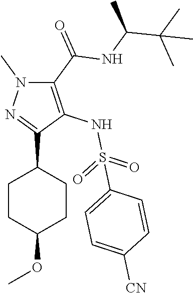 [SMILES: CO[C@H]1CC[C@@H](c2nn(C)c(C(=O)N[C@@H](C)C(C)(C)C)c2NS(=O)(=O)c2ccc(C#N)cc2)CC1] | 21 |
| 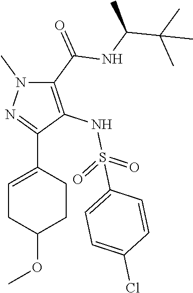 [SMILES: COC1CC=C(c2nn(C)c(C(=O)N[C@@H](C)C(C)(C)C)c2NS(=O)(=O)c2ccc(Cl)cc2)CC1] | 22 |
| 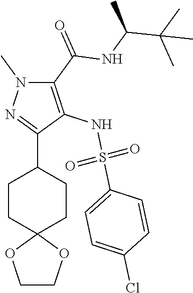 [SMILES: C[C@H](NC(=O)c1c(NS(=O)(=O)c2ccc(Cl)cc2)c(C2CCC3(CC2)OCCO3)nn1C)C(C)(C)C] | 23 |
| 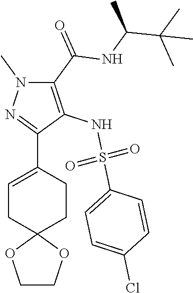 [SMILES: C[C@H](NC(=O)c1c(NS(=O)(=O)c2ccc(Cl)cc2)c(C2=CCC3(CC2)OCCO3)nn1C)C(C)(C)C] | 24 |
| 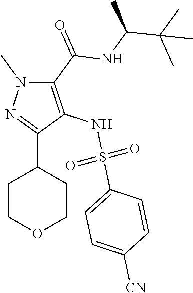 [SMILES: C[C@H](NC(=O)c1c(NS(=O)(=O)c2ccc(C#N)cc2)c(C2CCOCC2)nn1C)C(C)(C)C] | 25 |
| 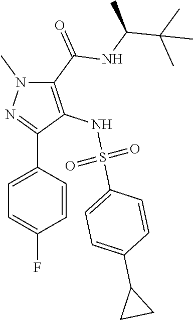 [SMILES: C[C@H](NC(=O)c1c(NS(=O)(=O)c2ccc(C3CC3)cc2)c(-c2ccc(F)cc2)nn1C)C(C)(C)C] | 26 |
| 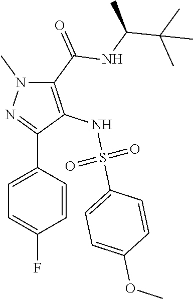 [SMILES: COc1ccc(S(=O)(=O)Nc2c(-c3ccc(F)cc3)nn(C)c2C(=O)N[C@@H](C)C(C)(C)C)cc1] | 27 |
| 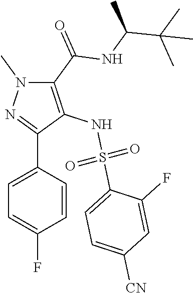 [SMILES: C[C@H](NC(=O)c1c(NS(=O)(=O)c2ccc(C#N)cc2F)c(-c2ccc(F)cc2)nn1C)C(C)(C)C] | 28 |
| 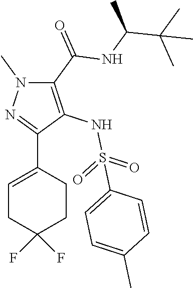 [SMILES: Cc1ccc(S(=O)(=O)Nc2c(C3=CCC(F)(F)CC3)nn(C)c2C(=O)N[C@@H](C)C(C)(C)C)cc1] | 29 |
| 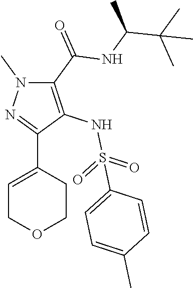 [SMILES: Cc1ccc(S(=O)(=O)Nc2c(C3=CCOCC3)nn(C)c2C(=O)N[C@@H](C)C(C)(C)C)cc1] | 30 |
| 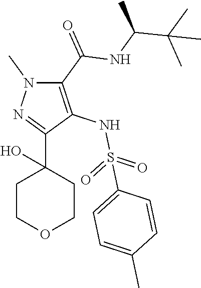 [SMILES: Cc1ccc(S(=O)(=O)Nc2c(C3(O)CCOCC3)nn(C)c2C(=O)N[C@@H](C)C(C)(C)C)cc1] | 31 |
| 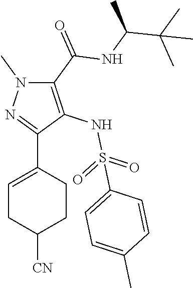 [SMILES: Cc1ccc(S(=O)(=O)Nc2c(C3=CCC(C#N)CC3)nn(C)c2C(=O)N[C@@H](C)C(C)(C)C)cc1] | 32 |
| 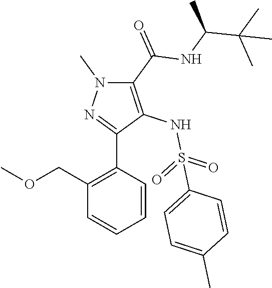 [SMILES: COCc1ccccc1-c1nn(C)c(C(=O)N[C@@H](C)C(C)(C)C)c1NS(=O)(=O)c1ccc(C)cc1] | 33 |
| 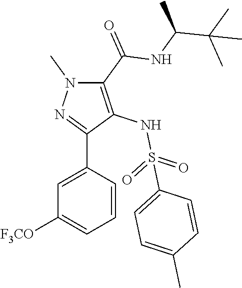 [SMILES: Cc1ccc(S(=O)(=O)Nc2c(-c3cccc(OC(F)(F)F)c3)nn(C)c2C(=O)N[C@@H](C)C(C)(C)C)cc1] | 34 |
| 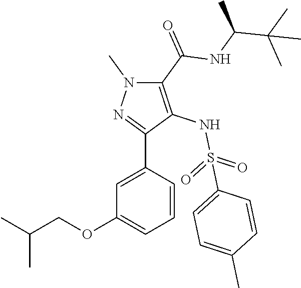 [SMILES: Cc1ccc(S(=O)(=O)Nc2c(-c3cccc(OCC(C)C)c3)nn(C)c2C(=O)N[C@@H](C)C(C)(C)C)cc1] | 35 |
| 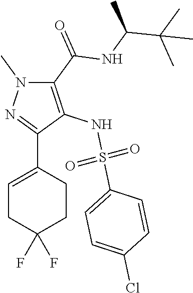 [SMILES: C[C@H](NC(=O)c1c(NS(=O)(=O)c2ccc(Cl)cc2)c(C2=CCC(F)(F)CC2)nn1C)C(C)(C)C] | 36 |
| 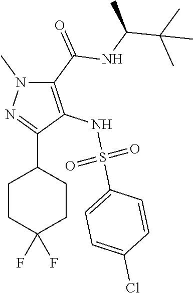 [SMILES: C[C@H](NC(=O)c1c(NS(=O)(=O)c2ccc(Cl)cc2)c(C2CCC(F)(F)CC2)nn1C)C(C)(C)C] | 37 |
| 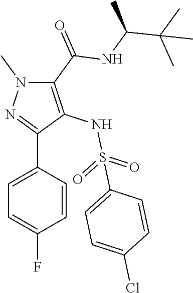 [SMILES: C[C@H](NC(=O)c1c(NS(=O)(=O)c2ccc(Cl)cc2)c(-c2ccc(F)cc2)nn1C)C(C)(C)C] | 38 |
| 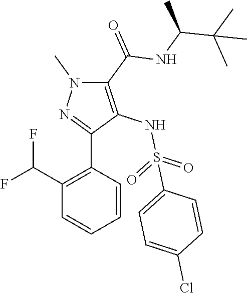 [SMILES: C[C@H](NC(=O)c1c(NS(=O)(=O)c2ccc(Cl)cc2)c(-c2ccccc2C(F)F)nn1C)C(C)(C)C] | 39 |
| 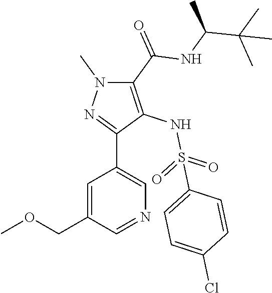 [SMILES: COCc1cncc(-c2nn(C)c(C(=O)N[C@@H](C)C(C)(C)C)c2NS(=O)(=O)c2ccc(Cl)cc2)c1] | 40 |
| 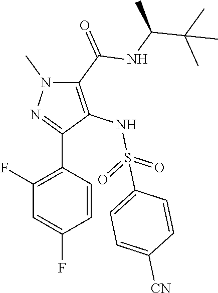 [SMILES: C[C@H](NC(=O)c1c(NS(=O)(=O)c2ccc(C#N)cc2)c(-c2ccc(F)cc2F)nn1C)C(C)(C)C] | 41 |
| 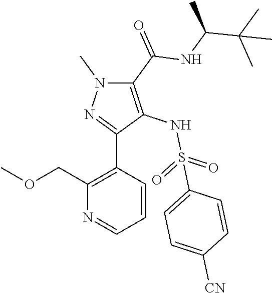 [SMILES: COCc1ncccc1-c1nn(C)c(C(=O)N[C@@H](C)C(C)(C)C)c1NS(=O)(=O)c1ccc(C#N)cc1] | 42 |
| 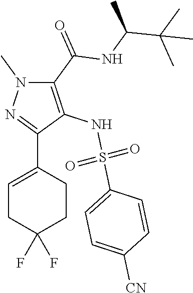 [SMILES: C[C@H](NC(=O)c1c(NS(=O)(=O)c2ccc(C#N)cc2)c(C2=CCC(F)(F)CC2)nn1C)C(C)(C)C] | 43 |
| 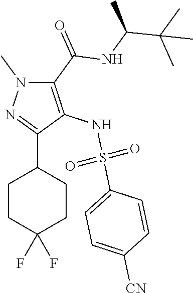 [SMILES: C[C@H](NC(=O)c1c(NS(=O)(=O)c2ccc(C#N)cc2)c(C2CCC(F)(F)CC2)nn1C)C(C)(C)C] | 44 |

Another aspect of the present disclosure provides pharmaceutical compositions comprising at least one compound selected from a compound of Formulae I, IIA, IIB, IIIA, IIIB, IVA, IVB, VA, VB, VIA, and VIB, Compounds 1 to 18, a tautomer thereof, a deuterated derivative of the compound or the tautomer, or a pharmaceutically acceptable salt of the foregoing, or a pharmaceutical composition comprising any of the foregoing, and at least one pharmaceutically acceptable carrier.

In some embodiments, the pharmaceutically acceptable carrier is selected from pharmaceutically acceptable vehicles and pharmaceutically acceptable adjuvants. In some embodiments, the pharmaceutically acceptable carrier is chosen from pharmaceutically acceptable fillers, disintegrants, surfactants, binders, and lubricants.

It will also be appreciated that a pharmaceutical composition of the present disclosure can be employed in combination therapies; that is, the pharmaceutical compositions described herein can further include an additional active pharmaceutical agent. Alternatively, a pharmaceutical composition comprising a compound selected from a compound of Formulae I, IIA, IIB, IIIA, IIIB, IVA, IVB, VA, VB, VIA, and VIB, Compounds 1 to 18, a tautomer thereof, a deuterated derivative of the compound or the tautomer, or a pharmaceutically acceptable salt of the foregoing, or a pharmaceutical composition comprising any of the foregoing can be administered as a separate composition concurrently with, prior to, or subsequent to, a composition comprising an additional active pharmaceutical agent.

As described above, the pharmaceutical compositions disclosed herein comprise a pharmaceutically acceptable carrier. The pharmaceutically acceptable carrier may be chosen from adjuvants and vehicles. The pharmaceutically acceptable carrier, as used herein, can be chosen, for example, from any and all solvents, diluents, other liquid vehicles, dispersion aids, suspension aids, surface active agents, isotonic agents, thickening agents, emulsifying agents, preservatives, solid binders, and lubricants, which are suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988 to 1999, Marcel Dekker, New York discloses various carriers used in formulating pharmaceutical compositions and known techniques for the preparation thereof. Except insofar as any conventional carrier is incompatible with the compounds of the present disclosure, such as by producing any undesirable biological effect or otherwise interacting in a deleterious manner with any other component(s) of the pharmaceutical composition, its use is contemplated to be within the scope of the present disclosure. Non-limiting examples of suitable pharmaceutically acceptable carriers include ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

### III. Methods of Treatment and Uses

In another aspect of the present disclosure, a compound, tautomer, deuterated derivative, or pharmaceutically acceptable salt as described herein, including a compound of Formulae I, IIA, IIB, IIIA, IIIB, IVA, IVB, VA, VB, VIA, and VIB, Compounds 1 to 18, a tautomer thereof, a deuterated derivative of the compound or the tautomer, or a pharmaceutically acceptable salt of the foregoing, or the pharmaceutical composition thereof, is for use in treating a disease, a disorder, or a condition mediated by the signaling of FPR1. In another aspect, disclosed herein is use of a compound, tautomer, deuterated derivative, or pharmaceutically acceptable salt as described herein, including a compound of Formulae I, IIA, IIB, IIIA, IIIB, IVA, IVB, VA, VB, VIA, and VIB, Compounds 1 to 18, a tautomer thereof, a deuterated derivative of the compound or the tautomer, or a pharmaceutically acceptable salt of the foregoing, or the pharmaceutical composition thereof, for the manufacture of a medicament for treating a disease, a disorder, or a condition mediated by the signaling of FPR1. In yet another aspect, disclosed herein is a method of treating a disease, a disorder, or a condition mediated by the signaling of FPR1 in a subject, comprising administering a therapeutically effective amount of a compound, tautomer, deuterated derivative, or pharmaceutically acceptable salt as described herein, including a compound of Formulae I, IIA, IIB, IIIA, IIIB, IVA, IVB, VA, VB, VIA, and VIB, Compounds 1 to 18, a tautomer thereof, a deuterated derivative of the compound or the tautomer, or a pharmaceutically acceptable salt of the foregoing, or the pharmaceutical composition thereof.

In some embodiments, the disease, the disorder, or the condition is related to the central nervous system (CNS). In some embodiment, the disease, the disorder, or the condition is selected from stroke, dementia, Alzheimer's disease, Parkinson's disease, Picks disease, fronto-temporal dementia, vascular dementia, normal pressure hydrocephalus, epilepsy, seizure disorder, amyotrophic lateral sclerosis (ALS), spinal motor atrophies, Tay-Sach's, Sandoff disease, familial spastic paraplegia, spinocerebellar ataxia (SCA), Friedrich's ataxia, Wilson's disease, Menke's Sx, cerebral autosomal dominant arteriopathy with subcortical infarcts (CADASIL); spinal muscular atrophy, muscular dystrophies, Charcot Marie Tooth diseases, neurofibromatosis, von-Hippel Lindau, Fragile X, spastic paraplesia, tuberous sclerosis, Wardenburg syndrome, dystonias, benign essential tremor, tardive dystonia, tardive dyskinesia, Tourette's syndrome, ataxic syndromes, Shy Drager, Olivopontoicerebellar degeneration, striatonigral degenration, Gullian Barre syndrome, causalgia, complex regional pain syndrome types I and II, diabetic neuropathy, and alcoholic neuropathy, trigeminal neuropathy, trigeminal neuralgia, Menier's syndrome, glossopharangela neuralgia, dysphagia, dysphonia, cranial nerve palsies, myelopethies, traumatic brain injury, traumatic spinal injury, radiation brain injury, multiple sclerosis, post-menengitis syndrome, prion diseases, myelities, radiculitis, diabetes associated with dysproteinemias, transthyretin-induced neuropathies, neuropathy associated with HIV, neuropathy associated with Lyme disease, neuropathy associated with herpes zoster, carpal tunnel syndrome, tarsal tunnel syndrome, amyloid-induced neuropathies, leprous neuropathy, Bell's palsy, compression neuropathies, sarcoidosis-induced neuropathy, polyneuritis cranialis, heavy metal induced neuropathy, transition metal-induced neuropathy, drug-induced neuropathy, axonic brain damage, encephalopathies, chronic fatigue syndrome, and a malignant glioma.

In one embodiment, the disease, the disorder, or the condition is stroke (thrombotic, embolic, thromboembolic, hemorrhagic, venoconstrictive, and venous). In one embodiment, the disease, the disorder, or the condition is traumatic brain injury. In one embodiment, the disease, the disorder, or the condition is a malignant glioma. In one embodiment, the malignant glioma is selected from glioblastoma, anaplastic astrocytoma, anaplastic oligdendroglioma, anaplastic oligoastrocytoma, anaplastic ependymoma, and anaplastic ganglioglioma. In one embodiment, the malignant glioma is glioblastoma.

In another aspect of the present disclosure, a compound, tautomer, deuterated derivative, or pharmaceutically acceptable salt as described herein, including a compound of Formulae I, IIA, IIB, IIIA, IIIB, IVA, IVB, VA, VB, VIA, and VIB, Compounds 1 to 18, a tautomer thereof, a deuterated derivative of the compound or the tautomer, or a pharmaceutically acceptable salt of the foregoing, or the pharmaceutical composition thereof, is for use in modulating FPR1 activity. In another aspect, disclosed herein is use of a compound, tautomer, deuterated derivative, or pharmaceutically acceptable salt as described herein, including a compound of Formulae I, IIA, IIB, IIIA, IIIB, IVA, IVB, VA, VB, VIA, and VIB, Compounds 1 to 18, a tautomer thereof, a deuterated derivative of the compound or the tautomer, or a pharmaceutically acceptable salt of the foregoing, or the pharmaceutical composition thereof, for the manufacture of a medicament for modulating FPR1 activity. In yet another aspect, disclosed herein is a method of modulating FPR1 activity, comprising administering a therapeutically effective amount of a compound, tautomer, deuterated derivative, or pharmaceutically acceptable salt as described herein to a subject, including a compound of Formulae I, IIA, IIB, IIIA, IIIB, IVA, IVB, VA, VB, VIA, and VIB, Compounds 1 to 18, a tautomer thereof, a deuterated derivative of the compound or the tautomer, or a pharmaceutically acceptable salt of the foregoing, or the pharmaceutical composition thereof. In yet another aspect, disclosed herein is a method of modulating FPR1 activity, comprising contacting said FPR1 a compound, tautomer, deuterated derivative, or pharmaceutically acceptable salt as described herein to a subject, including a compound of Formulae I, IIA, IIB, IIIA, IIIB, IVA, IVB, VA, VB, VIA, and VIB, Compounds 1 to 18, a tautomer thereof, a deuterated derivative of the compound or the tautomer, or a pharmaceutically acceptable salt of the foregoing, or the pharmaceutical composition thereof.

A compound of Formulae I, IIA, IIB, IIIA, IIIB, IVA, IVB, VA, VB, VIA, and VIB, Compounds 1 to 18, a tautomer thereof, a deuterated derivative of the compound or the tautomer, or a pharmaceutically acceptable salt of the foregoing, or the pharmaceutical composition thereof may be administered once daily, twice daily, or three times daily, for example, for the treatment of a disease, a disorder, or a condition mediated by the signaling of FPR1.

In some embodiments, 2 mg to 1500 mg or 5 mg to 1000 mg of a compound of Formulae I, IIA, IIB, IIIA, IIIB, IVA, IVB, VA, VB, VIA, and VIB, Compounds 1 to 18, a tautomer thereof, a deuterated derivative of the compound or the tautomer, or a pharmaceutically acceptable salt of the foregoing, or the pharmaceutical composition thereof are administered once daily, twice daily, or three times daily.

A compound of Formulae I, IIA, IIB, IIIA, IIIB, IVA, IVB, VA, VB, VIA, and VIB, Compounds 1 to 18, a tautomer thereof, a deuterated derivative of the compound or the tautomer, or a pharmaceutically acceptable salt of the foregoing, or the pharmaceutical composition thereof may be administered, for example, by oral, parenteral, sublingual, topical, rectal, nasal, buccal, vaginal, transdermal, patch, pump administration or via an implanted reservoir, and the pharmaceutical compositions would be formulated accordingly. Parenteral administration includes intravenous, intraperitoneal, subcutaneous, intramuscular, transepithelial, nasal, intrapulmonary, intrathecal, rectal and topical modes of administration. Parenteral administration can be by continuous infusion over a selected period of time. Other forms of administration contemplated in the present disclosure are as described in International Patent Application Nos. WO 2013/075083, WO 2013/075084, WO 2013/078320, WO 2013/120104, WO 2014/124418, WO 2014/151142, and WO 2015/023915.

Useful dosages or a therapeutically effective amount of a compound or pharmaceutically acceptable salt thereof as described herein can be determined by comparing their in vitro activity and in vivo activity in animal models. Methods for the extrapolation of effective dosages in mice and other animals, to humans are known to the art; for example, see U.S. Pat. No. 4,938,949.

One of ordinary skill in the art would recognize that, when an amount of compound is disclosed, the relevant amount of a pharmaceutically acceptable salt form of the compound is an amount equivalent to the concentration of the free base of the compound. The amounts of the compounds, pharmaceutically acceptable salts, solvates, and deuterated derivatives disclosed herein are based upon the free base form of the reference compound. For example, “1000 mg of at least one compound chosen from compounds of Formula I and pharmaceutically acceptable salts thereof” includes 1000 mg of compound of Formula I) and a concentration of a pharmaceutically acceptable salt of compounds of Formula I equivalent to 1000 mg of compounds of Formula I.

### Non-Limiting Exemplary Embodiments

1. A compound of Formula (I):

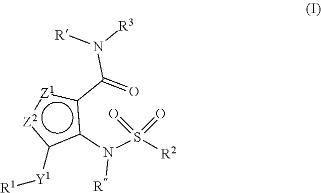

a tautomer thereof, a deuterated derivative of the compound or the tautomer, or a pharmaceutically acceptable salt the foregoing, wherein:
   (i) each Z¹ and Z² is independently chosen from O, S, N, NR⁴, C(R⁴)₂, and CR⁴, and at least one of Z¹ and Z² is O, S, N, or NR⁴;
      wherein R⁴ is chosen from hydrogen, linear, branched, and cyclic alkyl groups, heterocyclic groups, aryl groups, and heteroaryl groups;
   (ii) Y¹ is absent or chosen from a bond, O, S, or NR⁵;
      wherein R⁵ is chosen from hydrogen, linear, branched, and cyclic alkyl groups, heterocyclic groups, aryl groups, and heteroaryl groups;
   (iii) R¹ is chosen from linear, branched, and cyclic alkyl groups, alkenyl groups, heterocyclic groups, aryl groups, and heteroaryl groups; or R¹ and Z², together with the atoms to which they are attached, form a cycloalkyl group, a heterocyclic group, an aryl group, or a heteroaryl group;
   (iv) each R² and R³ is independently chosen from linear, branched, and cyclic alkyl groups, heterocyclic groups, aryl groups, and heteroaryl groups;
   (v) each R′ and R″ is independently chosen from hydrogen, linear, branched, and cyclic alkyl groups, heterocyclic groups, aryl groups, and heteroaryl groups;
wherein the linear, branched, and cyclic alkyl groups, heterocyclic groups, aryl groups, and heteroaryl groups are optionally substituted with at least one group chosen from the following groups:
   halogen groups,
   hydroxy,
   thiol,
   amino,
   cyano,
   C₁-C₆ linear, branched, and cyclic alkyl groups,
   C₂-C₆ linear, branched, and cyclic alkenyl groups,
   C₁-C₆ linear, branched, and cyclic hydroxyalkyl groups,
   C₁-C₆ linear, branched, and cyclic alkoxy groups,
   C₁-C₆ linear, branched, and cyclic thioalkyl groups,
   C₁-C₆ linear, branched, and cyclic haloalkyl groups,
   C₁-C₆ linear, branched, and cyclic haloaminoalkyl groups,
   C₁-C₆ linear, branched, and cyclic halothioalkyl groups,
   C₁-C₆ linear, branched, and cyclic haloalkoxy groups,
   benzyloxy, benzylamino, and benzylthio groups,
   3 to 6-membered heterocycloalkenyl groups,
   3 to 6-membered heterocyclic groups, and
   5 and 6-membered heteroaryl groups.
2. The compound, tautomer, deuterated derivative, or pharmaceutically acceptable salt of embodiment 1, wherein R¹ is chosen from cyclic alkyl groups, heterocyclic groups, aryl groups, and heteroaryl groups; R² is an aryl group; and R³ is chosen from linear and branched alkyl groups.
3. The compound, tautomer, deuterated derivative, or pharmaceutically acceptable salt of embodiment 1 or 2, wherein R¹ is chosen from optionally substituted 5 and 6-membered aryl groups.
4. The compound, tautomer, deuterated derivative, or pharmaceutically acceptable salt of embodiment 3, wherein R¹ is chosen from 5 and 6-membered aryl groups substituted with at least one halogen group.
5. The compound, tautomer, deuterated derivative, or pharmaceutically acceptable salt of embodiment 4, wherein R¹ is chosen from 5 and 6-membered aryl groups substituted with at least one fluoro.
6. The compound, tautomer, deuterated derivative, or pharmaceutically acceptable salt of embodiment 4, wherein R¹ is chosen from 5 and 6-membered aryl groups substituted with at least one chloro.
7. The compound, tautomer, deuterated derivative, or pharmaceutically acceptable salt of embodiment 1 or 2, wherein R¹ is chosen from optionally substituted 5 and 6-membered heteroaryl groups.
8. The compound, tautomer, deuterated derivative, or pharmaceutically acceptable salt of embodiment 7, wherein R¹ is chosen from 5 and 6-membered heteroaryl groups substituted with at least one halogen group.
9. The compound, tautomer, deuterated derivative, or pharmaceutically acceptable salt of embodiment 8, wherein R¹ is chosen from 5 and 6-membered heteroaryl groups substituted with at least one fluoro.
10. The compound, tautomer, deuterated derivative, or pharmaceutically acceptable salt of embodiment 8, wherein R¹ is chosen from 5 and 6-membered heteroaryl groups substituted with at least one chloro.
11. The compound, tautomer, deuterated derivative, or pharmaceutically acceptable salt of embodiment 1 or 2, wherein R¹ is chosen from optionally substituted 3 to 6-membered cyclic alkyl groups.
12. The compound, tautomer, deuterated derivative, or pharmaceutically acceptable salt of embodiment 11, wherein R¹ is chosen from 3 to 6-membered cyclic alkyl groups substituted with at least one alkoxy group.
13. The compound, tautomer, deuterated derivative, or pharmaceutically acceptable salt of embodiment 12, wherein R¹ is chosen from 3 to 6-membered cyclic alkyl groups substituted with at least one methoxy group.
14. The compound, tautomer, deuterated derivative, or pharmaceutically acceptable salt of embodiment 1 or 2, wherein R¹ is chosen from optionally substituted 3 to 6-membered carbocyclic, optionally substituted alkenyl groups, and optionally substituted heterocyclic groups.
15. The compound, tautomer, deuterated derivative, or pharmaceutically acceptable salt of any one of embodiments 1 to 14, wherein R² is chosen from optionally substituted 5 and 6-membered heteroaryl groups.
16. The compound, tautomer, deuterated derivative, or pharmaceutically acceptable salt of any one of embodiments 1 to 15, wherein R² is chosen from 5 and 6-membered heteroaryl groups substituted with at least one halogen group.
17. The compound, tautomer, deuterated derivative, or pharmaceutically acceptable salt of embodiment 16, wherein R² is chosen from 5 and 6-membered heteroaryl groups substituted with at least one fluoro.
18. The compound, tautomer, deuterated derivative, or pharmaceutically acceptable salt of embodiment 16, wherein R² is chosen from 5 and 6-membered heteroaryl groups substituted with at least one chloro.
19. The compound, tautomer, deuterated derivative, or pharmaceutically acceptable salt of any one of embodiments 1 to 15, wherein R² is chosen from 5 and 6-membered heteroaryl groups substituted with at least one cyano.
20. The compound, tautomer, deuterated derivative, or pharmaceutically acceptable salt of any one of embodiments 1 to 19, wherein R² is chosen from 5 and 6-membered heteroaryl groups substituted with at least one group chosen from C₁-C₆ linear, C₃-C₆ branched, and C₃-C₆ cyclic alkyl groups.
21. The compound, tautomer, deuterated derivative, or pharmaceutically acceptable salt of any one of embodiments 1 to 20, wherein R³ is chosen from optionally substituted C₂-C₁₀ linear and C₂-C₁₀ branched alkyl groups.
22. The compound, tautomer, deuterated derivative, or pharmaceutically acceptable salt of embodiment 21, wherein R³ is chosen from methyl, ethyl, propyl, isopropyl, butyl, iso-butyl, sec-butyl, pentyl, iso-pentyl, sec-pentyl, neo-pentyl, and 1,2,2-trimethylpropyl.
23. A compound of Formula (IIA):

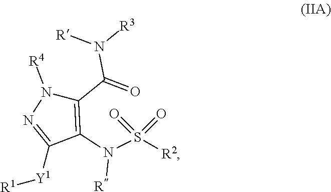

a tautomer thereof, a deuterated derivative of the compound or the tautomer, or a pharmaceutically acceptable salt of the foregoing, wherein:
   (i) Y¹ is absent or is chosen from a bond, O, S, or NR⁵;
      wherein R⁵ is chosen from hydrogen, linear, branched, and cyclic alkyl groups, heterocyclic groups, aryl groups, and heteroaryl groups;
   (ii) R¹ is chosen from linear, branched, and cyclic alkyl groups, alkenyl groups, heterocyclic groups, aryl groups, and heteroaryl groups;
   (iii) each R² and R³ is independently chosen from linear, branched, and cyclic alkyl groups, heterocyclic groups, aryl groups, and heteroaryl groups;
   (iv) R⁴ is chosen from hydrogen, linear, branched, and cyclic alkyl groups, heterocyclic groups, aryl groups, and heteroaryl groups;
   (v) each R′ and R″ is independently chosen from hydrogen, linear, branched, and cyclic alkyl groups, heterocyclic groups, aryl groups, and heteroaryl groups;
   wherein the linear, branched, and cyclic alkyl groups, heterocyclic groups, aryl groups, and heteroaryl groups are optionally substituted with at least one group chosen from the following groups:
      halogen groups,
      hydroxy,
      thiol,
      amino,
      cyano,
      C₁-C₆ linear, branched, and cyclic alkyl groups,
      C₂-C₆ linear, branched, and cyclic alkenyl groups,
      C₁-C₆ linear, branched, and cyclic hydroxyalkyl groups,
      C₁-C₆ linear, branched, and cyclic alkoxy groups,
      C₁-C₆ linear, branched, and cyclic thioalkyl groups,
      C₁-C₆ linear, branched, and cyclic haloalkyl groups,
      C₁-C₆ linear, branched, and cyclic haloaminoalkyl groups,
      C₁-C₆ linear, branched, and cyclic halothioalkyl groups,
      C₁-C₆ linear, branched, and cyclic haloalkoxy groups,
      benzyloxy, benzylamino, and benzylthio groups,
      3 to 6-membered heterocycloalkenyl groups,
      3 to 6-membered heterocyclic groups, and
      5 and 6-membered heteroaryl groups.
24. A compound of Formula (IIB):

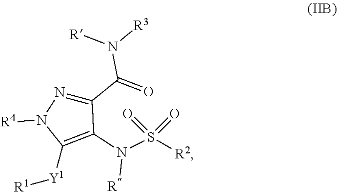

a tautomer thereof, a deuterated derivative of the compound or the tautomer, or a pharmaceutically acceptable salt of the foregoing, wherein:
   (i) Y¹ is absent or is chosen from a bond, O, S, or NR⁵;
      wherein R⁵ is chosen from hydrogen, linear, branched, and cyclic alkyl groups, heterocyclic groups, aryl groups, and heteroaryl groups;
   (ii) R¹ is chosen from linear, branched, and cyclic alkyl groups, alkenyl groups, heterocyclic groups, aryl groups, and heteroaryl groups; or R¹ and NR⁴, together with the atoms to which they are attached, form a cycloalkyl group, a heterocyclic group, an aryl group, or a heteroaryl group;
   (iii) each R² and R³ is independently chosen from linear, branched, and cyclic alkyl groups, heterocyclic groups, aryl groups, and heteroaryl groups;
   (iv) R⁴ is chosen from hydrogen, linear, branched, and cyclic alkyl groups, heterocyclic groups, aryl groups, and heteroaryl groups;
   (v) each R′ and R″ is independently chosen from hydrogen, linear, branched, and cyclic alkyl groups, heterocyclic groups, aryl groups, and heteroaryl groups;
   wherein the linear, branched, and cyclic alkyl groups, heterocyclic groups, aryl groups, and heteroaryl groups are optionally substituted with at least one group chosen from the following groups:
      halogen groups,
      hydroxy,
      thiol,
      amino,
      cyano,
      C₁-C₆ linear, branched, and cyclic alkyl groups,
      C₂-C₆ linear, branched, and cyclic alkenyl groups,
      C₁-C₆ linear, branched, and cyclic hydroxyalkyl groups,
      C₁-C₆ linear, branched, and cyclic alkoxy groups,
      C₁-C₆ linear, branched, and cyclic thioalkyl groups,
      C₁-C₆ linear, branched, and cyclic haloalkyl groups,
      C₁-C₆ linear, branched, and cyclic haloaminoalkyl groups,
      C₁-C₆ linear, branched, and cyclic halothioalkyl groups,
      C₁-C₆ linear, branched, and cyclic haloalkoxy groups,
      benzyloxy, benzylamino, and benzylthio groups,
      3 to 6-membered heterocycloalkenyl groups,
      3 to 6-membered heterocyclic groups, and
      5 and 6-membered heteroaryl groups.
25. The compound, tautomer, deuterated derivative, or pharmaceutically acceptable salt of embodiment 23 or 24, wherein R¹ is chosen from cyclic alkyl groups, heterocyclic groups, aryl groups, and heteroaryl groups; R² is an aryl group; and R³ is chosen from linear and branched alkyl groups.
26. The compound, tautomer, deuterated derivative, or pharmaceutically acceptable salt of embodiment 23 or 24, wherein R¹ is chosen from optionally substituted 5 and 6-membered aryl groups.
27. The compound, tautomer, deuterated derivative, or pharmaceutically acceptable salt of embodiment 26, wherein R¹ is chosen from 5 and 6-membered aryl groups substituted with at least one halogen group.
28. The compound, tautomer, deuterated derivative, or pharmaceutically acceptable salt of embodiment 27, wherein R¹ is chosen from 5 and 6-membered aryl groups substituted with at least one fluoro.
29. The compound, tautomer, deuterated derivative, or pharmaceutically acceptable salt of embodiment 27, wherein R¹ is chosen from 5 and 6-membered aryl groups substituted with at least one chloro.
30. The compound, tautomer, deuterated derivative, or pharmaceutically acceptable salt of embodiment 23 or 24, wherein R¹ is chosen from optionally substituted 5 and 6-membered heteroaryl groups.
31. The compound, tautomer, deuterated derivative, or pharmaceutically acceptable salt of embodiment 30, wherein R¹ is chosen from 5 and 6-membered heteroaryl groups substituted with at least one halogen group.
32. The compound, tautomer, deuterated derivative, or pharmaceutically acceptable salt of embodiment 31, wherein R¹ is chosen from 5 and 6-membered heteroaryl groups substituted with at least one fluoro.
33. The compound, tautomer, deuterated derivative, or pharmaceutically acceptable salt of embodiment 31, wherein R¹ is chosen from 5 and 6-membered heteroaryl groups substituted with at least one chloro.
34. The compound, tautomer, deuterated derivative, or pharmaceutically acceptable salt of embodiment 23 or 24, wherein R¹ is chosen from optionally substituted 3 to 6-membered cyclic alkyl groups.
35. The compound, tautomer, deuterated derivative, or pharmaceutically acceptable salt of embodiment 34, wherein R¹ is chosen from 3 to 6-membered cyclic alkyl groups substituted with at least one alkoxy group.
36. The compound, tautomer, deuterated derivative, or pharmaceutically acceptable salt of embodiment 35, wherein R¹ is chosen from 3 to 6-membered cyclic alkyl group substituted with at least one methoxy group.
37. The compound, tautomer, deuterated derivative, or pharmaceutically acceptable salt of embodiment 23 or 24, wherein R¹ is chosen from optionally substituted 3 to 6-membered heterocyclic groups.
38. The compound, tautomer, deuterated derivative, or pharmaceutically acceptable salt of any one of embodiments 23 to 37, wherein R² is chosen from optionally substituted 5 and 6-membered heteroaryl groups.
39. The compound, tautomer, deuterated derivative, or pharmaceutically acceptable salt of any one of embodiments 23 to 38, wherein R² is chosen from 5 and 6-membered heteroaryl groups substituted with at least one halogen group.
40. The compound, tautomer, deuterated derivative, or pharmaceutically acceptable salt of embodiment 40, wherein R² is chosen from 5 and 6-membered heteroaryl groups substituted with at least one fluoro.
41. The compound, tautomer, deuterated derivative, or pharmaceutically acceptable salt of embodiment 40, wherein R² is chosen from 5 and 6-membered heteroaryl groups substituted with at least one chloro.
42. The compound, tautomer, deuterated derivative, or pharmaceutically acceptable salt of any one of embodiments 23 to 38, wherein R² is chosen from 5 and 6-membered heteroaryl groups substituted with at least one cyano.
43. The compound, tautomer, deuterated derivative, or pharmaceutically acceptable salt of any one of embodiments 23 to 38, wherein R² is chosen from 5 and 6-membered heteroaryl groups substituted with at least one group chosen from C₁-C₆ linear, C₃-C₆ branched, and C₃-C₆ cyclic alkyl groups.
44. The compound, tautomer, deuterated derivative, or pharmaceutically acceptable salt of any one of embodiments 23 to 43, wherein R³ is chosen from optionally substituted C₁-C₁₀ linear and C₂-C₁₀ branched alkyl groups.
45. The compound, tautomer, deuterated derivative, or pharmaceutically acceptable salt of embodiment 44, wherein R³ is chosen from methyl, ethyl, propyl, isopropyl, butyl, iso-butyl, sec-butyl, pentyl, iso-pentyl, sec-pentyl, neo-pentyl, and 1,2,2-trimethylpropyl.
46. The compound, tautomer, deuterated derivative, or pharmaceutically acceptable salt of any one of embodiments 23 to 45, wherein R⁴ is chosen from optionally substituted C₁-C₁₀ linear, C₂-C₁₀ branched alkyl groups, and C₃-C₆ cyclic alkyl groups.
47. The compound, tautomer, deuterated derivative, or pharmaceutically acceptable salt of embodiment 45, wherein R⁴ is chosen from methyl, ethyl, propyl, isopropyl, cyclopropyl, butyl, iso-butyl, sec-butyl, cyclobutyl, pentyl, iso-pentyl, sec-pentyl, neo-pentyl, 1,2,2-trimethylpropyl, cyclopentyl, and cyclohexyl.
48. A compound of Formula (IIIA):

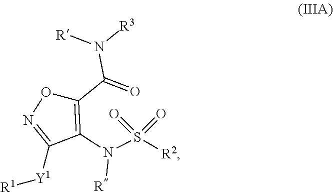

a tautomer thereof, a deuterated derivative of the compound or the tautomer, or a pharmaceutically acceptable salt of the foregoing, wherein:
   (i) Y¹ is absent or is chosen from a bond, O, S, or NR⁵;
      wherein R⁵ is chosen from hydrogen, linear, branched, and cyclic alkyl groups, heterocyclic groups, aryl groups, and heteroaryl groups;
   (ii) R¹ is chosen from linear, branched, and cyclic alkyl groups, alkenyl groups, heterocyclic groups, aryl groups, and heteroaryl groups;
   (iii) each R² and R³ is independently chosen from linear, branched, and cyclic alkyl groups, heterocyclic groups, aryl groups, and heteroaryl groups;
   (iv) each R′ and R″ is independently chosen from hydrogen, linear, branched, and cyclic alkyl groups, heterocyclic groups, aryl groups, and heteroaryl groups;
   wherein the linear, branched, and cyclic alkyl groups, heterocyclic groups, aryl groups, and heteroaryl groups are optionally substituted with at least one group chosen from the following groups:
      halogen groups,
      hydroxy,
      thiol,
      amino,
      cyano,
      C₁-C₆ linear, branched, and cyclic alkyl groups,
      C₂-C₆ linear, branched, and cyclic alkenyl groups,
      C₁-C₆ linear, branched, and cyclic hydroxyalkyl groups,
      C₁-C₆ linear, branched, and cyclic alkoxy groups,
      C₁-C₆ linear, branched, and cyclic thioalkyl groups,
      C₁-C₆ linear, branched, and cyclic haloalkyl groups,
      C₁-C₆ linear, branched, and cyclic haloaminoalkyl groups,
      C₁-C₆ linear, branched, and cyclic halothioalkyl groups,
      C₁-C₆ linear, branched, and cyclic haloalkoxy groups,
      benzyloxy, benzylamino, and benzylthio groups,
      3 to 6-membered heterocycloalkenyl groups,
      3 to 6-membered heterocyclic groups, and
      5 and 6-membered heteroaryl groups.
49. A compound of Formula (IIIB):

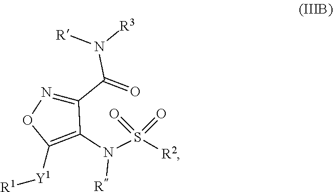

a tautomer thereof, a deuterated derivative of the compound or the tautomer, or a pharmaceutically acceptable salt of the foregoing, wherein:
   (i) Y¹ is absent or is chosen from a bond, O, S, or NR⁵;
      wherein R⁵ is chosen from hydrogen, linear, branched, and cyclic alkyl groups, heterocyclic groups, aryl groups, and heteroaryl groups;
   (ii) R¹ is chosen from linear, branched, and cyclic alkyl groups, alkenyl groups, heterocyclic groups, aryl groups, and heteroaryl groups;
   (iii) each R² and R³ is independently chosen from linear, branched, and cyclic alkyl groups, heterocyclic groups, aryl groups, and heteroaryl groups;
   (iv) each R′ and R″ is independently chosen from hydrogen, linear, branched, and cyclic alkyl groups, heterocyclic groups, aryl groups, and heteroaryl groups;
   wherein the linear, branched, and cyclic alkyl groups, heterocyclic groups, aryl groups, and heteroaryl groups are optionally substituted with at least one group chosen from the following groups:
      halogen groups,
      hydroxy,
      thiol,
      amino,
      cyano,
      C₁-C₆ linear, branched, and cyclic alkyl groups,
      C₂-C₆ linear, branched, and cyclic alkenyl groups,
      C₁-C₆ linear, branched, and cyclic hydroxyalkyl groups,
      C₁-C₆ linear, branched, and cyclic alkoxy groups,
      C₁-C₆ linear, branched, and cyclic thioalkyl groups,
      C₁-C₆ linear, branched, and cyclic haloalkyl groups,
      C₁-C₆ linear, branched, and cyclic haloaminoalkyl groups,
      C₁-C₆ linear, branched, and cyclic halothioalkyl groups,
      C₁-C₆ linear, branched, and cyclic haloalkoxy groups,
      benzyloxy, benzylamino, and benzylthio groups,
      3 to 6-membered heterocycloalkenyl groups,
      3 to 6-membered heterocyclic groups, and
      5 and 6-membered heteroaryl groups.
50. The compound, tautomer, deuterated derivative, or pharmaceutically acceptable salt of embodiment 48 or 49, wherein R¹ is chosen from cyclic alkyl groups, heterocyclic groups, aryl groups, and heteroaryl groups; R² is an aryl group; and R³ is chosen from linear and branched alkyl groups.
51. The compound, tautomer, deuterated derivative, or pharmaceutically acceptable salt of embodiment 48 or 49, wherein R¹ is chosen from optionally substituted 5 and 6-membered aryl groups.
52. The compound, tautomer, deuterated derivative, or pharmaceutically acceptable salt of embodiment 51, wherein R¹ is chosen from 5 and 6-membered aryl groups substituted with at least one halogen group.
53. The compound, tautomer, deuterated derivative, or pharmaceutically acceptable salt of embodiment 52, wherein R¹ is chosen from 5 and 6-membered aryl groups substituted with at least one fluoro.
54. The compound, tautomer, deuterated derivative, or pharmaceutically acceptable salt of embodiment 52, wherein R¹ is chosen from 5 and 6-membered aryl groups substituted with at least one chloro.
55. The compound, tautomer, deuterated derivative, or pharmaceutically acceptable salt of embodiment 48 or 49, wherein R¹ is chosen from optionally substituted 5 and 6-membered heteroaryl groups.
56. The compound, tautomer, deuterated derivative, or pharmaceutically acceptable salt of embodiment 55, wherein R¹ is chosen from 5 and 6-membered heteroaryl groups substituted with at least one halogen group.
57. The compound, tautomer, deuterated derivative, or pharmaceutically acceptable salt of embodiment 56, wherein R¹ is chosen from 5 and 6-membered heteroaryl groups substituted with at least one fluoro.
58. The compound, tautomer, deuterated derivative, or pharmaceutically acceptable salt of embodiment 56, wherein R¹ is chosen from 5 and 6-membered heteroaryl groups substituted with at least one chloro.
59. The compound, tautomer, deuterated derivative, or pharmaceutically acceptable salt of embodiment 48 or 49, wherein R¹ is chosen from optionally substituted 3 to 6-membered cyclic alkyl groups.
60. The compound, tautomer, deuterated derivative, or pharmaceutically acceptable salt of embodiment 59, wherein R¹ is chosen from 3 to 6-membered cyclic alkyl groups substituted with at least one alkoxy group.
61. The compound, tautomer, deuterated derivative, or pharmaceutically acceptable salt of embodiment 60, wherein R¹ is chosen from 3 to 6-membered cyclic alkyl groups substituted with at least one methoxy group.
62. The compound, tautomer, deuterated derivative, or pharmaceutically acceptable salt of embodiment 48 or 49, wherein R¹ is chosen from optionally substituted 3 to 6-membered heterocyclic groups.
63. The compound, tautomer, deuterated derivative, or pharmaceutically acceptable salt of any one of embodiments 48 to 62, wherein R² is chosen from optionally substituted 5 and 6-membered heteroaryl groups.
64. The compound, tautomer, deuterated derivative, or pharmaceutically acceptable salt of any one of embodiments 48 to 63, wherein R² is chosen from 5 and 6-membered heteroaryl groups substituted with at least one halogen group.
65. The compound, tautomer, deuterated derivative, or pharmaceutically acceptable salt of embodiment 64, wherein R² is chosen from 5 and 6-membered heteroaryl groups substituted with at least one fluoro.
66. The compound, tautomer, deuterated derivative, or pharmaceutically acceptable salt of embodiment 64, wherein R² is chosen from 5 and 6-membered heteroaryl groups substituted with at least one chloro.
67. The compound, tautomer, deuterated derivative, or pharmaceutically acceptable salt of any one of embodiments 48 to 62, wherein R² is chosen from 5 and 6-membered heteroaryl groups substituted with at least one cyano.
68. The compound, tautomer, deuterated derivative, or pharmaceutically acceptable salt of any one of embodiments 48 to 62, wherein R² is chosen from 5 and 6-membered heteroaryl groups substituted with at least one group chosen from C₁-C₆ linear, C₃-C₆ branched, and C₃-C₆ cyclic alkyl groups.
69. The compound, tautomer, deuterated derivative, or pharmaceutically acceptable salt of any one of embodiments 48 to 68, wherein R³ is chosen from optionally substituted C₁-C₁₀ linear and C₂-C₁₀ branched alkyl groups.
70. The compound, tautomer, deuterated derivative, or pharmaceutically acceptable salt of embodiment 69, wherein R³ is chosen from methyl, ethyl, propyl, isopropyl, butyl, iso-butyl, sec-butyl, pentyl, iso-pentyl, sec-pentyl, neo-pentyl, and 1,2,2-trimethylpropyl.
71. The compound, tautomer, deuterated derivative, or pharmaceutically acceptable salt of any one of embodiments 48 to 70, wherein R⁴ is chosen from optionally substituted C₁-C₁₀ linear, C₂-C₁₀ branched alkyl groups, and C₃-C₆ cyclic alkyl groups.
72. The compound, tautomer, deuterated derivative, or pharmaceutically acceptable salt of embodiment 71, wherein R⁴ is chosen from methyl, ethyl, propyl, isopropyl, cyclopropyl, butyl, iso-butyl, sec-butyl, cyclobutyl, pentyl, iso-pentyl, sec-pentyl, neo-pentyl, 1,2,2-trimethylpropyl, cyclopentyl, and cyclohexyl.
73. A compound of Formula (IVA):

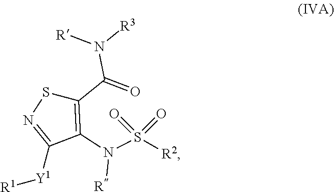

a tautomer thereof, a deuterated derivative of the compound or the tautomer, or a pharmaceutically acceptable salt of the foregoing, wherein:
   (i) Y¹ is absent or is chosen from a bond, O, S, or NR⁵;
      wherein R⁵ is chosen from hydrogen, linear, branched, and cyclic alkyl groups, heterocyclic groups, aryl groups, and heteroaryl groups;
   (ii) R¹ is chosen from linear, branched, and cyclic alkyl groups, alkenyl groups, heterocyclic groups, aryl groups, and heteroaryl groups;
   (iii) each R² and R³ is independently chosen from linear, branched, and cyclic alkyl groups, heterocyclic groups, aryl groups, and heteroaryl groups;
   (iv) each R′ and R″ is independently chosen from hydrogen, linear, branched, and cyclic alkyl groups, heterocyclic groups, aryl groups, and heteroaryl groups;
   wherein the linear, branched, and cyclic alkyl groups, heterocyclic groups, aryl groups, and heteroaryl groups are optionally substituted with at least one group chosen from the following groups:
      halogen groups,
      hydroxy,
      thiol,
      amino,
      cyano,
      C₁-C₆ linear, branched, and cyclic alkyl groups,
      C₂-C₆ linear, branched, and cyclic alkenyl groups,
      C₁-C₆ linear, branched, and cyclic hydroxyalkyl groups,
      C₁-C₆ linear, branched, and cyclic alkoxy groups,
      C₁-C₆ linear, branched, and cyclic thioalkyl groups,
      C₁-C₆ linear, branched, and cyclic haloalkyl groups,
      C₁-C₆ linear, branched, and cyclic haloaminoalkyl groups,
      C₁-C₆ linear, branched, and cyclic halothioalkyl groups,
      C₁-C₆ linear, branched, and cyclic haloalkoxy groups,
      benzyloxy, benzylamino, and benzylthio groups,
      3 to 6-membered heterocycloalkenyl groups,
      3 to 6-membered heterocyclic groups, and
      5 and 6-membered heteroaryl groups.
74. A compound of Formula (IVB):

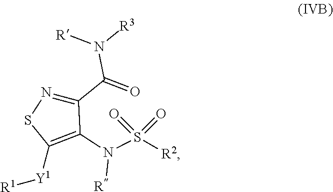

a tautomer thereof, a deuterated derivative of the compound or the tautomer, or a pharmaceutically acceptable salt of the foregoing, wherein:
   (i) Y¹ is absent or is chosen from a bond, O, S, or NR⁵;
      wherein R⁵ is chosen from hydrogen, linear, branched, and cyclic alkyl groups, heterocyclic groups, aryl groups, and heteroaryl groups;
   (ii) R¹ is chosen from linear, branched, and cyclic alkyl groups, alkenyl groups, heterocyclic groups, aryl groups, and heteroaryl groups;
   (iii) each R² and R³ is independently chosen from linear, branched, and cyclic alkyl groups, heterocyclic groups, aryl groups, and heteroaryl groups;
   (iv) each R′ and R″ is independently chosen from hydrogen, linear, branched, and cyclic alkyl groups, heterocyclic groups, aryl groups, and heteroaryl groups;
   wherein the linear, branched, and cyclic alkyl groups, heterocyclic groups, aryl groups, and heteroaryl groups are optionally substituted with at least one group chosen from the following groups:
      halogen groups,
      hydroxy,
      thiol,
      amino,
      cyano,
      C₁-C₆ linear, branched, and cyclic alkyl groups,
      C₂-C₆ linear, branched, and cyclic alkenyl groups,
      C₁-C₆ linear, branched, and cyclic hydroxyalkyl groups,
      C₁-C₆ linear, branched, and cyclic alkoxy groups,
      C₁-C₆ linear, branched, and cyclic thioalkyl groups,
      C₁-C₆ linear, branched, and cyclic haloalkyl groups,
      C₁-C₆ linear, branched, and cyclic haloaminoalkyl groups,
      C₁-C₆ linear, branched, and cyclic halothioalkyl groups,
      C₁-C₆ linear, branched, and cyclic haloalkoxy groups,
      benzyloxy, benzylamino, and benzylthio groups,
      3 to 6-membered heterocycloalkenyl groups,
      3 to 6-membered heterocyclic groups, and
      5 and 6-membered heteroaryl groups.
75. The compound, tautomer, deuterated derivative, or pharmaceutically acceptable salt of embodiment 73 or 74, wherein R¹ is chosen from cyclic alkyl groups, heterocyclic groups, aryl groups, and heteroaryl groups; R² is an aryl group; and R³ is chosen from linear and branched, alkyl groups.
76. The compound, tautomer, deuterated derivative, or pharmaceutically acceptable salt of embodiment 73 or 74, wherein R¹ is chosen from optionally substituted 5 and 6-membered aryl groups.
77. The compound, tautomer, deuterated derivative, or pharmaceutically acceptable salt of embodiment 76, wherein R¹ is chosen from 5 and 6-membered aryl groups substituted with at least one halogen group.
78. The compound, tautomer, deuterated derivative, or pharmaceutically acceptable salt of embodiment 77, wherein R¹ is chosen from 5 and 6-membered aryl groups substituted with at least one fluoro.
79. The compound, tautomer, deuterated derivative, or pharmaceutically acceptable salt of embodiment 77, wherein R¹ is chosen from 5 and 6-membered aryl groups substituted with at least one chloro.
80. The compound, tautomer, deuterated derivative, or pharmaceutically acceptable salt of embodiment 73 or 74, wherein R¹ is chosen from optionally substituted 5 and 6-membered heteroaryl groups.
81. The compound, tautomer, deuterated derivative, or pharmaceutically acceptable salt of embodiment 80, wherein R¹ is chosen from 5 and 6-membered heteroaryl groups substituted with at least one halogen group.
82. The compound, tautomer, deuterated derivative, or pharmaceutically acceptable salt of embodiment 81, wherein R¹ is chosen from 5 and 6-membered heteroaryl groups substituted with at least one fluoro.
83. The compound, tautomer, deuterated derivative, or pharmaceutically acceptable salt of embodiment 81, wherein R¹ is chosen from 5 and 6-membered heteroaryl groups substituted with at least one chloro.
84. The compound, tautomer, deuterated derivative, or pharmaceutically acceptable salt of embodiment 73 or 74, wherein R¹ is chosen from optionally substituted 3 to 6-membered cyclic alkyl groups.
85. The compound, tautomer, deuterated derivative, or pharmaceutically acceptable salt of embodiment 84, wherein R¹ is chosen from 3 to 6-membered cyclic alkyl groups substituted with at least one alkoxy group.
86. The compound, tautomer, deuterated derivative, or pharmaceutically acceptable salt of embodiment 85, wherein R¹ is chosen from 3 to 6-membered cyclic alkyl groups substituted with at least one methoxy group.
87. The compound, tautomer, deuterated derivative, or pharmaceutically acceptable salt of embodiment 73 or 74, wherein R¹ is chosen from optionally substituted 3 to 6-membered heterocyclic groups.
88. The compound, tautomer, deuterated derivative, or pharmaceutically acceptable salt of any one of embodiments 73 to 87, wherein R² is chosen from optionally substituted 5 and 6-membered heteroaryl groups.
89. The compound, tautomer, deuterated derivative, or pharmaceutically acceptable salt of any one of embodiments 73 to 88, wherein R² is chosen from 5 and 6-membered heteroaryl groups substituted with at least one halogen group.
90. The compound, tautomer, deuterated derivative, or pharmaceutically acceptable salt of embodiment 89, wherein R² is chosen from 5 and 6-membered heteroaryl groups substituted with at least one fluoro.
91. The compound, tautomer, deuterated derivative, or pharmaceutically acceptable salt of embodiment 89, wherein R² is chosen from 5 and 6-membered heteroaryl groups substituted with at least one chloro.
92. The compound, tautomer, deuterated derivative, or pharmaceutically acceptable salt of any one of embodiments 73 to 87, wherein R² is chosen from 5 and 6-membered heteroaryl groups substituted with at least one cyano.
93. The compound, tautomer, deuterated derivative, or pharmaceutically acceptable salt of any one of embodiments 73 to 87, wherein R² is chosen from 5 and 6-membered heteroaryl groups substituted with at least one group chosen from C₁-C₆ linear, C₃-C₆ branched, and C₃-C₆ cyclic alkyl groups.
94. The compound, tautomer, deuterated derivative, or pharmaceutically acceptable salt of any one of embodiments 73 to 93, wherein R³ is chosen from optionally substituted C₁-C₁₀ linear and C₂-C₁₀ branched alkyl groups.
95. The compound, tautomer, deuterated derivative, or pharmaceutically acceptable salt of embodiment 94, wherein R³ is chosen from methyl, ethyl, propyl, isopropyl, butyl, iso-butyl, sec-butyl, pentyl, iso-pentyl, sec-pentyl, neo-pentyl, and 1,2,2-trimethylpropyl.
96. A compound of Formula (VA):

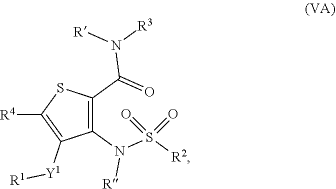

a tautomer thereof, a deuterated derivative of the compound or the tautomer, or a pharmaceutically acceptable salt of the foregoing, wherein:
   (i) Y¹ is absent or is chosen from a bond, O, S, or NR⁵;
      wherein R⁵ is chosen from hydrogen, linear, branched, and cyclic alkyl groups, heterocyclic groups, aryl groups, and heteroaryl groups;
   (ii) R¹ is chosen from linear, branched, and cyclic alkyl groups, alkenyl groups, heterocyclic groups, aryl groups, and heteroaryl groups;
   (iii) each R² and R³ is independently chosen from linear, branched, and cyclic alkyl groups, heterocyclic groups, aryl groups, and heteroaryl groups;
   (iv) each R′ and R″ is independently chosen from hydrogen, linear, branched, and cyclic alkyl groups, heterocyclic groups, aryl groups, and heteroaryl groups;
   wherein the linear, branched, and cyclic alkyl groups, heterocyclic groups, aryl groups, and heteroaryl groups are optionally substituted with at least one group chosen from the following groups:
      halogen groups,
      hydroxy,
      thiol,
      amino,
      cyano,
      C₁-C₆ linear, branched, and cyclic alkyl groups,
      C₂-C₆ linear, branched, and cyclic alkenyl groups,
      C₁-C₆ linear, branched, and cyclic hydroxyalkyl groups,
      C₁-C₆ linear, branched, and cyclic alkoxy groups,
      C₁-C₆ linear, branched, and cyclic thioalkyl groups,
      C₁-C₆ linear, branched, and cyclic haloalkyl groups,
      C₁-C₆ linear, branched, and cyclic haloaminoalkyl groups,
      C₁-C₆ linear, branched, and cyclic halothioalkyl groups,
      C₁-C₆ linear, branched, and cyclic haloalkoxy groups,
      benzyloxy, benzylamino, and benzylthio groups,
      3 to 6-membered heterocycloalkenyl groups,
      3 to 6-membered heterocyclic groups, and
      5 and 6-membered heteroaryl groups.
97. A compound of Formula (VB):

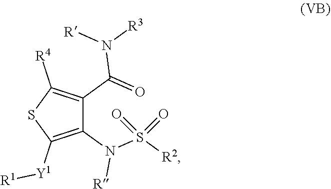

a tautomer thereof, a deuterated derivative of the compound or the tautomer, or a pharmaceutically acceptable salt of the foregoing, wherein:
   (i) Y¹ is absent or is chosen from a bond, O, S, or NR⁵;
      wherein R⁵ is chosen from hydrogen, linear, branched, and cyclic alkyl groups, heterocyclic groups, aryl groups, and heteroaryl groups;
   (ii) R¹ is chosen from linear, branched, and cyclic alkyl groups, alkenyl groups, heterocyclic groups, aryl groups, and heteroaryl groups;
   (iii) each R² and R³ is independently chosen from linear, branched, and cyclic alkyl groups, heterocyclic groups, aryl groups, and heteroaryl groups;
   (iv) each R′ and R″ is independently chosen from hydrogen, linear, branched, and cyclic alkyl groups, heterocyclic groups, aryl groups, and heteroaryl groups;
   wherein the linear, branched, and cyclic alkyl groups, heterocyclic groups, aryl groups, and heteroaryl groups are optionally substituted with at least one group chosen from the following groups:
      halogen groups,
      hydroxy,
      thiol,
      amino,
      cyano,
      C₁-C₆ linear, branched, and cyclic alkyl groups,
      C₂-C₆ linear, branched, and cyclic alkenyl groups,
      C₁-C₆ linear, branched, and cyclic hydroxyalkyl groups,
      C₁-C₆ linear, branched, and cyclic alkoxy groups,
      C₁-C₆ linear, branched, and cyclic thioalkyl groups,
      C₁-C₆ linear, branched, and cyclic haloalkyl groups,
      C₁-C₆ linear, branched, and cyclic haloaminoalkyl groups,
      C₁-C₆ linear, branched, and cyclic halothioalkyl groups,
      C₁-C₆ linear, branched, and cyclic haloalkoxy groups,
      benzyloxy, benzylamino, and benzylthio groups,
      3 to 6-membered heterocycloalkenyl groups,
      3 to 6-membered heterocyclic groups, and
      5 and 6-membered heteroaryl groups.
98. The compound, tautomer, deuterated derivative, or pharmaceutically acceptable salt of embodiment 96 or 97, wherein R¹ is chosen from cyclic alkyl groups, heterocyclic groups, aryl groups, and heteroaryl groups; R² is an aryl group; and R³ is chosen from linear or branched alkyl groups.
99. The compound, tautomer, deuterated derivative, or pharmaceutically acceptable salt of embodiment 96 or 97, wherein R¹ is chosen from optionally substituted 5 and 6-membered aryl groups.
100. The compound, tautomer, deuterated derivative, or pharmaceutically acceptable salt of embodiment 99, wherein R¹ is chosen from 5 and 6-membered aryl groups substituted with at least one halogen group.
101. The compound, tautomer, deuterated derivative, or pharmaceutically acceptable salt of embodiment 100, wherein R¹ is chosen from 5 and 6-membered aryl groups substituted with at least one fluoro.
102. The compound, tautomer, deuterated derivative, or pharmaceutically acceptable salt of embodiment 100, wherein R¹ is chosen from 5 and 6-membered aryl groups substituted with at least one chloro.
103. The compound, tautomer, deuterated derivative, or pharmaceutically acceptable salt of embodiment 96 or 97, wherein R¹ is chosen from optionally substituted 5 and 6-membered heteroaryl groups.
104. The compound, tautomer, deuterated derivative, or pharmaceutically acceptable salt of embodiment 103, wherein R¹ is chosen from 5 and 6-membered heteroaryl groups substituted with at least one halogen group.
105. The compound, tautomer, deuterated derivative, or pharmaceutically acceptable salt of embodiment 104, wherein R¹ is chosen from 5 and 6-membered heteroaryl groups substituted with at least one fluoro.
106. The compound, tautomer, deuterated derivative, or pharmaceutically acceptable salt of embodiment 104, wherein R¹ is chosen from 5 and 6-membered heteroaryl groups substituted with at least one chloro.
107. The compound, tautomer, deuterated derivative, or pharmaceutically acceptable salt of embodiment 96 or 97, wherein R¹ is chosen from optionally substituted 3 to 6-membered cyclic alkyl groups.
108. The compound, tautomer, deuterated derivative, or pharmaceutically acceptable salt of embodiment 107, wherein R¹ is chosen from 3 to 6-membered cyclic alkyl groups substituted with at least one alkoxy group.
109. The compound, tautomer, deuterated derivative, or pharmaceutically acceptable salt of embodiment 108, wherein R¹ is chosen from 3 to 6-membered cyclic alkyl groups substituted with at least one methoxy group.
110. The compound, tautomer, deuterated derivative, or pharmaceutically acceptable salt of embodiment 96 or 97, wherein R¹ is chosen from optionally substituted 3 to 6-membered heterocyclic groups.
111. The compound, tautomer, deuterated derivative, or pharmaceutically acceptable salt of any one of embodiments 96 to 110, wherein R² is chosen from optionally substituted 5 and 6-membered heteroaryl groups.
112. The compound, tautomer, deuterated derivative, or pharmaceutically acceptable salt of any one of embodiments 96 to 111, wherein R² is chosen from 5 and 6-membered heteroaryl groups substituted with at least one halogen group.
113. The compound, tautomer, deuterated derivative, or pharmaceutically acceptable salt of embodiment 112, wherein R² is chosen from 5 and 6-membered heteroaryl groups substituted with at least one fluoro.
114. The compound, tautomer, deuterated derivative, or pharmaceutically acceptable salt of embodiment 112, wherein R² is chosen from 5 and 6-membered heteroaryl groups substituted with at least one chloro.
115. The compound, tautomer, deuterated derivative, or pharmaceutically acceptable salt of any one of embodiments 96 to 110, wherein R² is chosen from 5 and 6-membered heteroaryl groups substituted with at least one cyano.
116. The compound, tautomer, deuterated derivative, or pharmaceutically acceptable salt of any one of embodiments 96 to 110, wherein R² is chosen from 5 and 6-membered heteroaryl groups substituted with at least one group chosen from C₁-C₆ linear, C₃-C₆ branched, and C₃-C₆ cyclic alkyl groups.
117. The compound, tautomer, deuterated derivative, or pharmaceutically acceptable salt of any one of embodiments 96 to 116, wherein R³ is chosen from optionally substituted C₁-C₁₀ linear and C₂-C₁₀ branched alkyl groups.
118. The compound, tautomer, deuterated derivative, or pharmaceutically acceptable salt of embodiment 117, wherein R³ is chosen from methyl, ethyl, propyl, isopropyl, butyl, iso-butyl, sec-butyl, pentyl, iso-pentyl, sec-pentyl, neo-pentyl, and 1,2,2-trimethylpropyl.
119. The compound, tautomer, deuterated derivative, or pharmaceutically acceptable salt of any one of embodiments 96 to 118, wherein R⁴ is chosen from optionally substituted C₁-C₁₀ linear, C₂-C₁₀ branched alkyl groups, and C₃-C₆ cyclic alkyl groups.
120. The compound, tautomer, deuterated derivative, or pharmaceutically acceptable salt of embodiment 119, wherein R⁴ is chosen from methyl, ethyl, propyl, isopropyl, cyclopropyl, butyl, iso-butyl, sec-butyl, cycloputyl, pentyl, iso-pentyl, sec-pentyl, neo-pentyl, 1,2,2-trimethylpropyl, cyclopentyl, and cyclohexyl.
121. A compound of Formula (VIA):

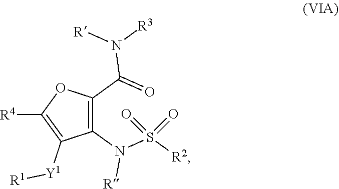

a tautomer thereof, a deuterated derivative of the compound or the tautomer, or a pharmaceutically acceptable salt of the foregoing, wherein:
   (i) Y¹ is absent or is chosen from a bond, O, S, or NR5;
      wherein R5 is chosen from hydrogen, linear, branched, and cyclic alkyl groups, heterocyclic groups, aryl groups, and heteroaryl groups;
   (ii) R¹ is chosen from linear, branched, and cyclic alkyl groups, alkenyl groups, heterocyclic groups, aryl groups, and heteroaryl groups;
   (iii) each R² and R³ is independently chosen from linear, branched, and cyclic alkyl groups, heterocyclic groups, aryl groups, and heteroaryl groups;
   (iv) each R′ and R″ is independently chosen from hydrogen, linear, branched, and cyclic alkyl groups, heterocyclic groups, aryl groups, and heteroaryl groups;
   wherein the linear, branched, and cyclic alkyl groups, heterocyclic groups, aryl groups, and heteroaryl groups are optionally substituted with at least one group chosen from the following groups:
      halogen groups,
      hydroxy,
      thiol,
      amino,
      cyano,
      C₁-C₆ linear, branched, and cyclic alkyl groups,
      C₂-C₆ linear, branched, and cyclic alkenyl groups,
      C₁-C₆ linear, branched, and cyclic hydroxyalkyl groups,
      C₁-C₆ linear, branched, and cyclic alkoxy groups,
      C₁-C₆ linear, branched, and cyclic thioalkyl groups,
      C₁-C₆ linear, branched, and cyclic haloalkyl groups,
      C₁-C₆ linear, branched, and cyclic haloaminoalkyl groups,
      C₁-C₆ linear, branched, and cyclic halothioalkyl groups,
      C₁-C₆ linear, branched, and cyclic haloalkoxy groups,
      benzyloxy, benzylamino, and benzylthio groups,
      3 to 6-membered heterocycloalkenyl groups,
      3 to 6-membered heterocyclic groups, and
      5 and 6-membered heteroaryl groups.
122. A compound of Formula (VIB):

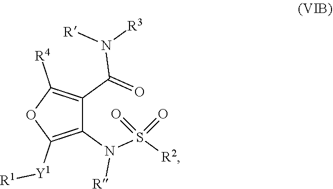

a tautomer thereof, a deuterated derivative of the compound or the tautomer, or a pharmaceutically acceptable salt of the foregoing, wherein:
   (i) Y¹ is absent or is chosen from a bond, O, S, or NR⁵;
      wherein R⁵ is chosen from hydrogen, linear, branched, and cyclic alkyl groups, heterocyclic groups, aryl groups, and heteroaryl groups;
   (ii) R¹ is chosen from linear, branched, and cyclic alkyl groups, alkenyl groups, heterocyclic groups, aryl groups, and heteroaryl groups;
   (iii) each R² and R³ is independently chosen from linear, branched, and cyclic alkyl groups, heterocyclic groups, aryl groups, and heteroaryl groups;
   (iv) each R′ and R″ is independently chosen from hydrogen, linear, branched, and cyclic alkyl groups, heterocyclic groups, aryl groups, and heteroaryl groups;
   wherein the linear, branched, and cyclic alkyl groups, heterocyclic groups, aryl groups, and heteroaryl groups are optionally substituted with at least one group chosen from the following groups:
      halogen groups,
      hydroxy,
      thiol,
      amino,
      cyano,
      C₁-C₆ linear, branched, and cyclic alkyl groups,
      C₂-C₆ linear, branched, and cyclic alkenyl groups,
      C₁-C₆ linear, branched, and cyclic hydroxyalkyl groups,
      C₁-C₆ linear, branched, and cyclic alkoxy groups,
      C₁-C₆ linear, branched, and cyclic thioalkyl groups,
      C₁-C₆ linear, branched, and cyclic haloalkyl groups,
      C₁-C₆ linear, branched, and cyclic haloaminoalkyl groups,
      C₁-C₆ linear, branched, and cyclic halothioalkyl groups,
      C₁-C₆ linear, branched, and cyclic haloalkoxy groups,
      benzyloxy, benzylamino, and benzylthio groups,
      3 to 6-membered heterocycloalkenyl groups,
      3 to 6-membered heterocyclic groups, and
      5 and 6-membered heteroaryl groups.
123. The compound, tautomer, deuterated derivative, or pharmaceutically acceptable salt of embodiment 121 or 122, wherein R¹ is chosen from cyclic alkyl groups, heterocyclic groups, aryl groups, and heteroaryl groups; R² is an aryl group; and R³ is chosen from linear and branched alkyl groups.
124. The compound, tautomer, deuterated derivative, or pharmaceutically acceptable salt of embodiment 121 or 122, wherein R¹ is chosen from optionally substituted 5 and 6-membered aryl groups.
125. The compound, tautomer, deuterated derivative, or pharmaceutically acceptable salt of embodiment 124, wherein R¹ is chosen from 5 and 6-membered aryl groups substituted with at least one halogen group.
126. The compound, tautomer, deuterated derivative, or pharmaceutically acceptable salt of embodiment 125, wherein R¹ is chosen from 5 and 6-membered aryl groups substituted with at least one fluoro.
127. The compound, tautomer, deuterated derivative, or pharmaceutically acceptable salt of embodiment 125, wherein R¹ is chosen from 5 and 6-membered aryl groups substituted with at least one chloro.
128. The compound, tautomer, deuterated derivative, or pharmaceutically acceptable salt of embodiment 121 or 122, wherein R¹ is chosen from optionally substituted 5 and 6-membered heteroaryl groups.
129. The compound, tautomer, deuterated derivative, or pharmaceutically acceptable salt of embodiment 128, wherein R¹ is chosen from 5 and 6-membered heteroaryl groups substituted with at least one halogen group.
130. The compound, tautomer, deuterated derivative, or pharmaceutically acceptable salt of embodiment 129, wherein R¹ is chosen from 5 and 6-membered heteroaryl groups substituted with at least one fluoro.
131. The compound, tautomer, deuterated derivative, or pharmaceutically acceptable salt of embodiment 129, wherein R¹ is chosen from 5 and 6-membered heteroaryl group substituted with at least one chloro.
132. The compound, tautomer, deuterated derivative, or pharmaceutically acceptable salt of embodiment 121 or 122, wherein R¹ is chosen from optionally substituted 3 to 6-membered cyclic alkyl groups.
133. The compound, tautomer, deuterated derivative, or pharmaceutically acceptable salt of embodiment 132, wherein R¹ is chosen from 3 to 6-membered cyclic alkyl groups substituted with at least one alkoxy group.
134. The compound, tautomer, deuterated derivative, or pharmaceutically acceptable salt of embodiment 133, wherein R¹ is chosen from 3 to 6-membered cyclic alkyl groups substituted with at least one methoxy group.
135. The compound, tautomer, deuterated derivative, or pharmaceutically acceptable salt of embodiment 121 or 122, wherein R¹ is chosen from optionally substituted 3 to 6-membered heterocyclic groups.
136. The compound, tautomer, deuterated derivative, or pharmaceutically acceptable salt of any one of embodiments 121 to 135, wherein R² is chosen from optionally substituted 5 and 6-membered heteroaryl groups.
137. The compound, tautomer, deuterated derivative, or pharmaceutically acceptable salt of any one of embodiments 121 to 136, wherein R² is chosen from 5 and 6-membered heteroaryl groups substituted with at least one halogen group.
138. The compound, tautomer, deuterated derivative, or pharmaceutically acceptable salt of embodiment 137, wherein R² is chosen from 5 and 6-membered heteroaryl groups substituted with at least one fluoro.
139. The compound, tautomer, deuterated derivative, or pharmaceutically acceptable salt of embodiment 137, wherein R² is chosen from 5 and 6-membered heteroaryl groups substituted with at least one chloro.
140. The compound, tautomer, deuterated derivative, or pharmaceutically acceptable salt of any one of embodiments 121 to 136, wherein R² is chosen from 5 and 6-membered heteroaryl group substituted with at least one cyano.
141. The compound, tautomer, deuterated derivative, or pharmaceutically acceptable salt of any one of embodiments 121 to 135, wherein R² is chosen from 5 and 6-membered heteroaryl groups substituted with at least one group chosen from C₁-C₆ linear, C₃-C₆ branched, and C₃-C₆ cyclic alkyl groups.
142. The compound, tautomer, deuterated derivative, or pharmaceutically acceptable salt of any one of embodiments 121 to 141, wherein R³ is chosen from optionally substituted C₁-C₁₀ linear and C₂-C₁₀ branched alkyl groups.
143. The compound, tautomer, deuterated derivative, or pharmaceutically acceptable salt of embodiment 142, wherein R³ is chosen from methyl, ethyl, propyl, isopropyl, butyl, iso-butyl, sec-butyl, pentyl, iso-pentyl, sec-pentyl, neo-pentyl, and 1,2,2-trimethylpropyl.
144. The compound, tautomer, deuterated derivative, or pharmaceutically acceptable salt of any one of embodiments 121 to 143, wherein R⁴ is chosen from optionally substituted C₁-C₁₀ linear, C₂-C₁₀ branched alkyl groups, and C₃-C₆ cyclic alkyl groups.
145. The compound, tautomer, deuterated derivative, or pharmaceutically acceptable salt of embodiment 144, wherein R⁴ is chosen from methyl, ethyl, propyl, isopropyl, cyclopropyl, butyl, iso-butyl, sec-butyl, cyclobutyl, pentyl, iso-pentyl, sec-pentyl, neo-pentyl, 1,2,2-trimethylpropyl, cyclopentyl, and cyclohexyl.
146. A compound chosen from the 44 listed compounds set forth above, a tautomer thereof, a deuterated derivative of the compound or the tautomer, or a pharmaceutically acceptable salt of the foregoing.
147. A pharmaceutical composition comprising a compound, tautomer, deuterated derivative, or pharmaceutically acceptable salt according to any one of embodiments 1-146 and at least one pharmaceutically acceptable carrier.
148. A method for treating or alleviating a disease, a disorder or a condition mediated by the signaling of formyl peptide receptor 1 (FPR1), comprising administering to a subject in need thereof a therapeutically effective amount of a compound, tautomer, deuterated derivative, or pharmaceutically acceptable salt according to any one of the embodiments 1-146 or the pharmaceutical composition according to embodiment 147.
149. The method of embodiment 148, wherein the disease, the disorder, or the condition is related to the CNS and is chosen from stroke, dementia, Alzheimer's disease, Parkinson's disease, Picks disease, frontotemporal dementia, vascular dementia, normal pressure hydrocephalus, epilepsy, seizure disorder, amyotrophic lateral sclerosis (ALS), spinal motor atrophies, Tay-Sachs disease, Sandhoff disease, familial spastic paraplegia, spinocerebellar ataxia (SCA), Friedrich's ataxia, Wilson's disease, Menkes syndrome, cerebral autosomal dominant arteriopathy with subcortical infarcts (CADASTL), spinal muscular atrophy, muscular dystrophies, Charcot-Marie-Tooth disease, neurofibromatosis, Von Hippel-Lindau disease, Fragile X syndrome, spastic paraplegia, tuberous sclerosis, Waardenburg syndrome, dystonia, benign essential tremor, tardive dystonia, tardive dyskinesia, Tourette's syndrome, ataxic syndromes, Shy Drager syndrome, olivopontocerebellar degeneration, striatonigral degeneration, Guillain-Barre syndrome, causalgia, complex regional pain syndrome types I and II, diabetic neuropathy, and alcoholic neuropathy, trigeminal neuropathy, trigeminal neuralgia, Meniere's syndrome, glossopharyngeal neuralgia, dysphagia, dysphonia, cranial nerve palsies, myelopathy, traumatic brain injury, traumatic spinal injury, radiation brain injury, multiple sclerosis, post-meningitis syndrome, prion diseases, myelitis, radiculitis, diabetes associated with dysproteinemias, transthyretin-induced neuropathies, neuropathy associated with HIV, neuropathy associated with Lyme disease, neuropathy associated with herpes zoster, carpal tunnel syndrome, tarsal tunnel syndrome, amyloid-induced neuropathies, leprous neuropathy, Bell's palsy, compression neuropathies, sarcoidosis-induced neuropathy, polyneuritis cranialis, heavy metal induced neuropathy, transition metal-induced neuropathy, drug-induced neuropathy, axonic brain damage, encephalopathies, chronic fatigue syndrome, and a malignant glioma.
150. The method of embodiment 148 or 149, wherein the disease, the disorder, or the condition is stroke (thrombotic, embolic, thromboembolic, hemorrhagic, venoconstrictive, and venous).
151. The method of embodiment 148 or 149, wherein the disease, the disorder, or the condition is traumatic brain injury.
152. The method of embodiment 148 or 149, wherein the disease, the disorder, or the condition is a malignant glioma.
153. The method of embodiment 152, wherein the malignant glioma is chosen from glioblastoma, anaplastic astrocytoma, anaplastic oligodendroglioma anaplastic oligoastrocytoma, anaplastic ependymoma, and anaplastic ganglioglioma.
154. The method of embodiment 153, wherein the malignant glioma is glioblastoma.
155. The method of embodiment 148 or 149, wherein the disease, the disorder, or the condition is chosen from acute respiratory distress syndrome (ARDS), Allergic conjunctivitis (AC), and dry eye syndrome (DES).

### EXAMPLES

### Example 1. Synthesis of Compounds

To fully understand the present disclosure, the following examples are disclosed. It should be understood that these examples are for illustrative purposes only and are not to be construed as limiting the present disclosure in any manner.

All the specific and generic compounds, and the intermediates disclosed for making those compounds, are considered to be part of the present disclosure.

The compounds of the present disclosure may be made according to standard chemical practices or as described herein. Throughout the following synthetic schemes and in the descriptions for preparing compounds of Formulae (I), (II), (IIIa), (IIIb), and (IIc), Compounds 1 to 135, pharmaceutically acceptable salts of any of those compounds, solvates of any of the foregoing, and deuterated derivatives of any of the foregoing, the following abbreviations are used:

### Abbreviations

Boc₂O=di-tert-butyl dicarbonate
DCM=dichloromethane
DIEA=N,N-Diisopropylethylamine or N-ethyl-N-isopropyl-propan-2-amine
DMAP=dimethylamino pyridine
DMA=dimethyl acetamide
DME=dimethoxyethane
DMF=dimethylformamide
DMSO=dimethyl sulfoxide
EtOAc/EA=Ethyl Acetate
EtOH=ethanol
HOAc=acetic acid
KOAc=potassium acetate
LiHMDS=lithium bis(trimethylsilyl)amide
MeMgBr=methylmagnesium bromide
MeOH=methanol
NaOAc=sodium acetate
NBS=N-bromosuccinimide
Pd(dppf)₂Cl₂=[1,1′-Bis(diphenylphosphino)ferrocene]dichloropalladium(II)
PTSA=p-Toluenesulfonic acid monohydrate
rt=room (ambient) temperature
T3P=2,4,6-Tripropyl-1,3,5,2,4,6-trioxatriphosphorinane-2,4,6-trioxide
TEA=triethylamine
TFA=trifluoroacetic acid
THF=tetrahydrofuran
TsCl=p-toluene sulfonyl chloride

### Compound 1: (S)-4-((4-cyanophenyl)sulfonamido)-N-(3,3-dimethylbutan-2-yl)-3-(4-fluorophenyl)-1-methyl-1H-pyrazole-5-carboxamide

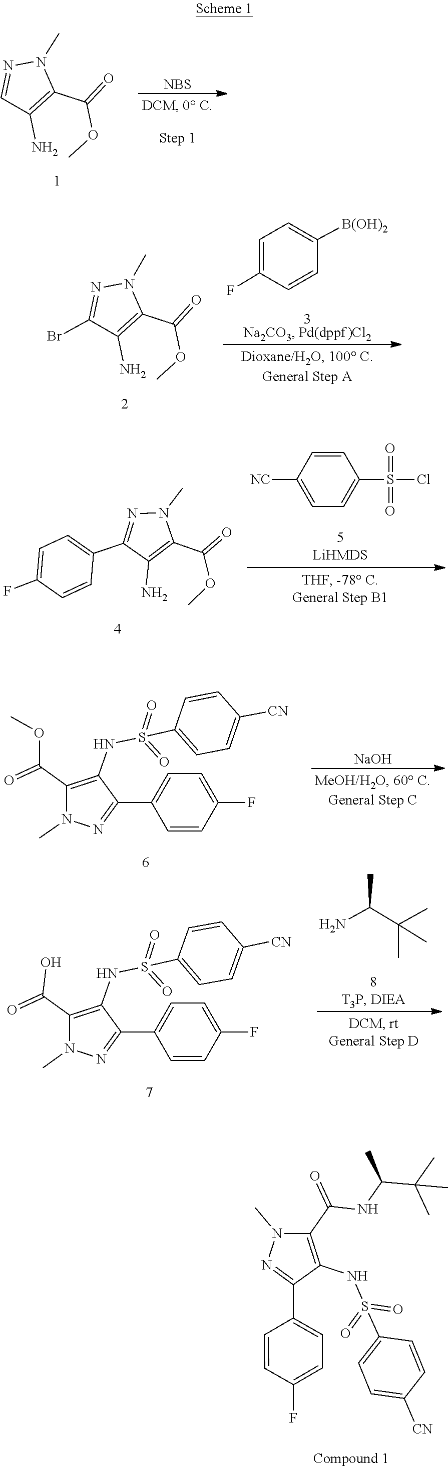

Step 1. Preparation of methyl 4-amino-3-bromo-1-methyl-1H-pyrazole-5-carboxylate: to a solution of methyl 4-amino-1-methyl-1H-pyrazole-5-carboxylate (1.0 g, 6.4 mmol) in DCM (20 mL) under N₂ was added NBS (1.37 g, 7.7 mmol) at 0° C. The resulting solution was stirred at 0° C. for 1 hr. The solvent was removed under reduced pressure, and the residue was purified by Combiflash (PE/EA=1:3) to give the product methyl 4-amino-3-bromo-1-methyl-1H-pyrazole-5-carboxylate as brown solid (0.96 g, 64%). Mass (m/z): 234.1, 236.1 [M+H]⁺.

General Step A. Preparation of methyl 4-amino-3-(4-fluorophenyl)-1-methyl-1H-pyrazole-5-carboxylate: to a solution of methyl 4-amino-3-bromo-1-methyl-1H-pyrazole-5-carboxylate (1.0 g, 4.3 mmol), (4-fluorophenyl)boronic acid (896 mg, 6.4 mmol) and Na₂CO₃ (1.35 g, 12.8 mmol) in the mixed solvent of dioxane and H₂O (33 mL, 10/1 (v/v)) was added Pd(dppf)Cl₂ (312 mg, 0.42 mmol) under N₂. The resulting mixture was stirred at 100° C. for 16 hrs. The solvent was removed under reduced pressure, and the residue was purified by Combiflash (PE/EA=1:1) to give the product methyl 4-amino-3-(4-fluorophenyl)-1-methyl-1H-pyrazole-5-carboxylate as yellow solid (856 mg, 80%). Mass (m/z): 250.1 [M+H]⁺.

General Step B1. Preparation of methyl 4-((4-cyanophenyl)sulfonamido)-3-(4-fluorophenyl)-1-methyl-1H-pyrazole-5-carboxylate: to a solution of 4-amino-3-(4-fluorophenyl)-1-methyl-1H-pyrazole-5-carboxylate (150 mg, 0.60 mmol) in THF (10 mL) was added LiHMDS (1.2 mL, 1 N solution in THF, 1.2 mmol) dropwise at −78° C. under N₂. The resulting solution was stirred at −78° C. for 1 hr, before the addition of 4-cyanobenzenesulfonyl chloride (182 mg, 0.90 mmol). The mixture was further stirred at −78° C. for 3 hrs, before the reaction was quenched with saturated NH₄Cl aqueous solution (20 mL). The aqueous media was extracted with EA (50 mL×3). The combined organic layers were washed with brine (30 mL×3), dried over anhydrous Na₂SO₄, and then filtered. The filtrate was concentrated under vacuum, and the residue was purified by flash column chromatography (PE/EA=1:1) to give the product methyl 4-((4-cyanophenyl)sulfonamido)-3-(4-fluorophenyl)-1-methyl-1H-pyrazole-5-carboxylate as white solid (164 mg, 65%). Mass (m/z): 437.0 [M+Na]+.

General Step C. Preparation of 4-((4-cyanophenyl)sulfonamido)-3-(4-fluorophenyl)-1-methyl-1H-pyrazole-5-carboxylic acid: to a solution of 4-((4-cyanophenyl)sulfonamido)-3-(4-fluorophenyl)-1-methyl-1H-pyrazole-5-carboxylate (100 mg, 0.24 mmol) in the mixed solvent of MeOH and H₂O (8 mL, 3:1 (v/v)) was added NaOH (96 mg, 2.4 mmol). The resulting mixture was stirred at 60° C. under N₂ for 16 hrs. The organic solvent was removed under reduced pressure. The aqueous media was acidified to pH 5-6 with 1 N HCl aqueous solution, and then extracted with EA (15 mL×3). The combined organic layers were washed with brine (10 mL×3), dried over Na₂SO₄, and then filtered. The filtrate was concentrated to dryness to give the product 4-((4-cyanophenyl)sulfonamido)-3-(4-fluorophenyl)-1-methyl-1H-pyrazole-5-carboxylic acid as white solid (73 mg, 75%). Mass (m/z): 401.0 [M+H]⁺.

General Step D. Preparation of (S)-4-((4-cyanophenyl)sulfonamido)-N-(3,3-dimethylbutan-2-yl)-3-(4-fluorophenyl)-1-methyl-1H-pyrazole-5-carboxamide (Compound 1): to a solution of 4-((4-cyanophenyl)sulfonamido)-3-(4-fluorophenyl)-1-methyl-1H-pyrazole-5-carboxylic acid (73 mg, 0.18 mmol) in DCM (10 mL) were added (S)-3,3-dimethylbutan-2-amine (27 mg, 0.27 mmol), DIEA (117 mg, 0.91 mmol) and T₃P (173 mg, 0.54 mmol) sequentially. The resulting solution was stirred at ambient temperature under N₂ for 3 hrs, and then partitioned with H₂O (50 mL). The mixture was extracted with DCM (50 mL×3). The combined organic layers were washed with brine (30 mL×3), dried over anhydrous Na₂SO₄, and then filtered. The filtrate was concentrated under vacuum, and the crude residue was purified by Combiflash (DCM/MeOH=10:1) to give the product (S)-4-((4-cyanophenyl)sulfonamido)-N-(3,3-dimethylbutan-2-yl)-3-(4-fluorophenyl)-1-methyl-1H-pyrazole-5-carboxamide (Compound 1) as white solid (54 mg, 61%). Mass (m z): 484.0 [M+H]⁺.

### Compound 2: 3-(4-fluorophenyl)-1-methyl-4-(phenylsulfonamido)-N-propyl-1H-pyrazole-5-carboxamide

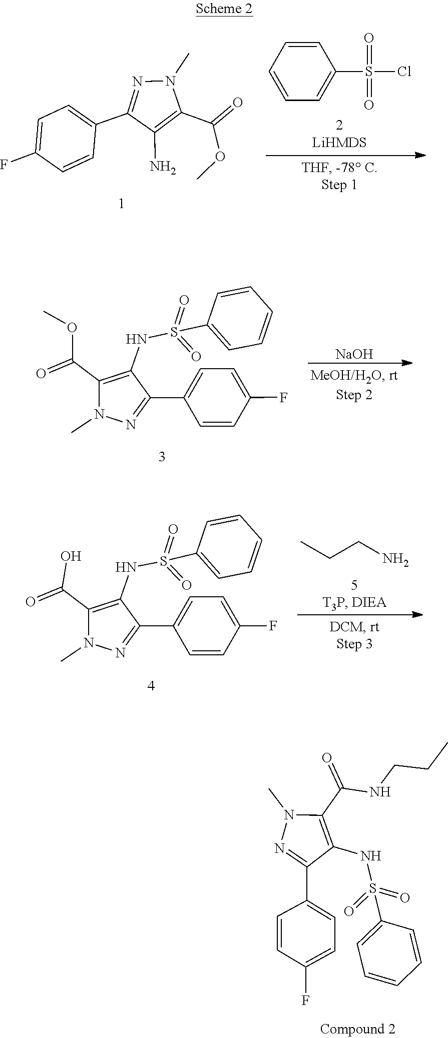

Step 1. Following General Step B1, methyl 3-(4-fluorophenyl)-1-methyl-4-(phenylsulfonamido)-1H-pyrazole-5-carboxylate was prepared as yellow solid (600 mg, 85%). Mass (m/z): 390.1 [M+H]⁺.

Step 2. Following General Step C, 3-(4-fluorophenyl)-1-methyl-4-(phenylsulfonamido)-1H-pyrazole-5-carboxylic acid was prepared as white solid (605 mg, 99%). Mass (m/z): 376.1 [M+H]⁺.

Step 3. Following General Step D, 3-(4-fluorophenyl)-1-methyl-4-(phenylsulfonamido)-N-propyl-1H-pyrazole-5-carboxamide (Compound 2) was prepared as white solid (57 mg, 51%). Mass (m/z): 417.0 [M+H]⁺.

### Compound 3: (S)—N-(sec-butyl)-3-(4-fluorophenyl)-1-methyl-4-(phenylsulfonamido)-1H-pyrazole-5-carboxamide

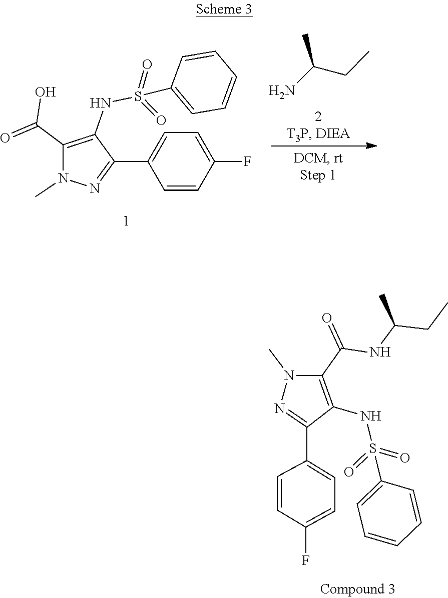

Step 1. Following General Step D, (S)—N-(sec-butyl)-3-(4-fluorophenyl)-1-methyl-4-(phenylsulfonamido)-1H-pyrazole-5-carboxamide (Compound 3) was prepared as white solid (39 mg, 35%). Mass (m/z): 431.0 [M+H]⁺.

### Compound 4: 3-(4-fluorophenyl)-N-(2-hydroxyethyl)-1-methyl-4-(phenylsulfonamido)-1H-pyrazole-5-carboxamide

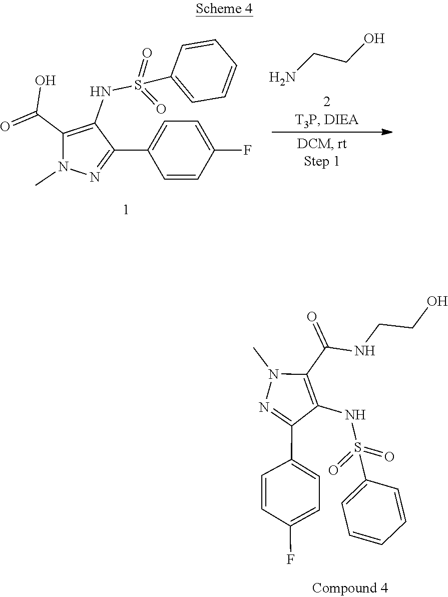

Step 1. Following General Step D, 3-(4-fluorophenyl)-N-(2-hydroxyethyl)-1-methyl-4-(phenylsulfonamido)-1H-pyrazole-5-carboxamide (Compound 4) was prepared as white solid (27 mg, 24%). Mass (m/z): 418.9 [M+H]⁺.

### Compound 5: (S)-4-((4-chlorophenyl)sulfonamido)-N-(3,3-dimethylbutan-2-yl)-1-methyl-3-(pyridin-4-yl)-1H-pyrazole-5-carboxamide

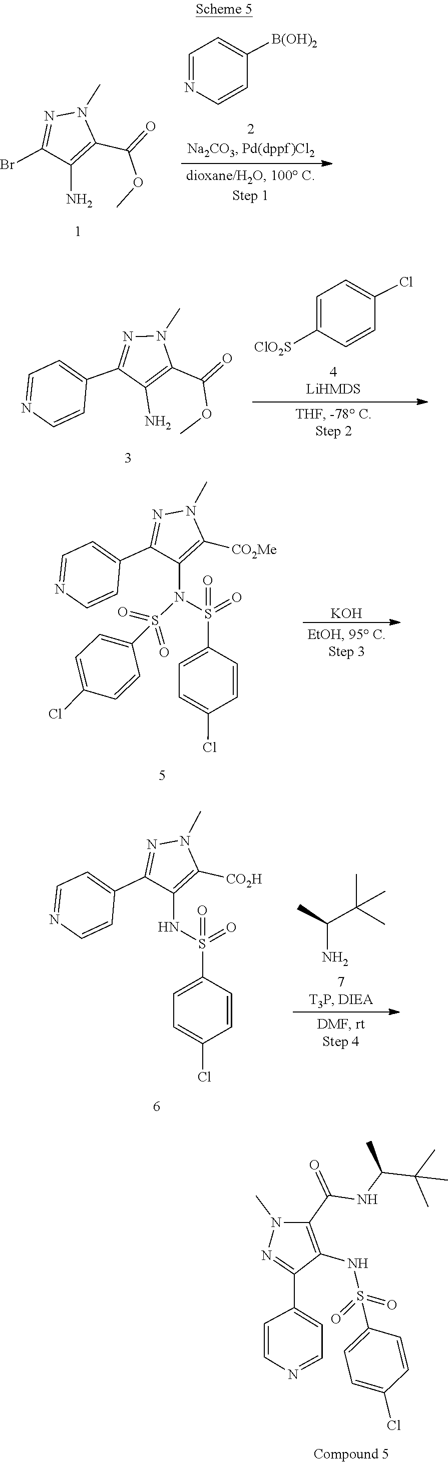

Step 1. Following General Step A, methyl 4-amino-1-methyl-3-(pyridin-4-yl)-1H-pyrazole-5-carboxylate was prepared as brown solid (456 mg, 85%). Mass (m/z): 233.1 [M+H]⁺.

Step 2. Following General Step B1, methyl 4-((4-chloro-N-((4-chlorophenyl)sulfonyl)phenyl)sulfonamido)-1-methyl-3-(pyridin-4-yl)-1H-pyrazole-5-carboxylate was prepared as brown solid (400 mg, 79%). Mass (m/z): 580.8 [M+H]⁺.

Step 3. Preparation of 4-((4-chlorophenyl)sulfonamido)-1-methyl-3-(pyridin-4-yl)-1H-pyrazole-5-carboxylic acid: to a solution of methyl 4-((4-chloro-N-((4-chlorophenyl)sulfonyl)phenyl)sulfonamido)-1-methyl-3-(pyridin-4-yl)-1H-pyrazole-5-carboxylate (400 mg, 0.68 mmol) in EtOH (10 mL) was added KOH (77 mg, 1.37 mmol). The resulting mixture was stirred at 95° C. under N₂ for 12 hrs, and then concentrated under reduced pressure. The residue was purified by preparative reverse-phase HPLC [Column: Gemini-C18, 150×21.2 mm, 5 um; Eluent: 10-50% MeCN in H₂O (0.1% TFA)] to give the product 4-((4-chlorophenyl)sulfonamido)-1-methyl-3-(pyridin-4-yl)-1H-pyrazole-5-carboxylic acid as white solid (50 mg, 18%). Mass (m/z): 392.8 [M+H]⁺.

Step 4. Following General Step D, (S)-4-((4-chlorophenyl)sulfonamido)-N-(3,3-dimethylbutan-2-yl)-1-methyl-3-(pyridin-4-yl)-1H-pyrazole-5-carboxamide (Compound 5) was prepared as white solid (55 mg, 90%). Mass (m/z): 475.6 [M+H]⁺.

### Compound 6: N—((S)-3,3-dimethylbutan-2-yl)-3-((1s,4R)-4-methoxycyclohexyl)-1-methyl-4-((4-methylphenyl)sulfonamido)-1H-pyrazole-5-carboxamide

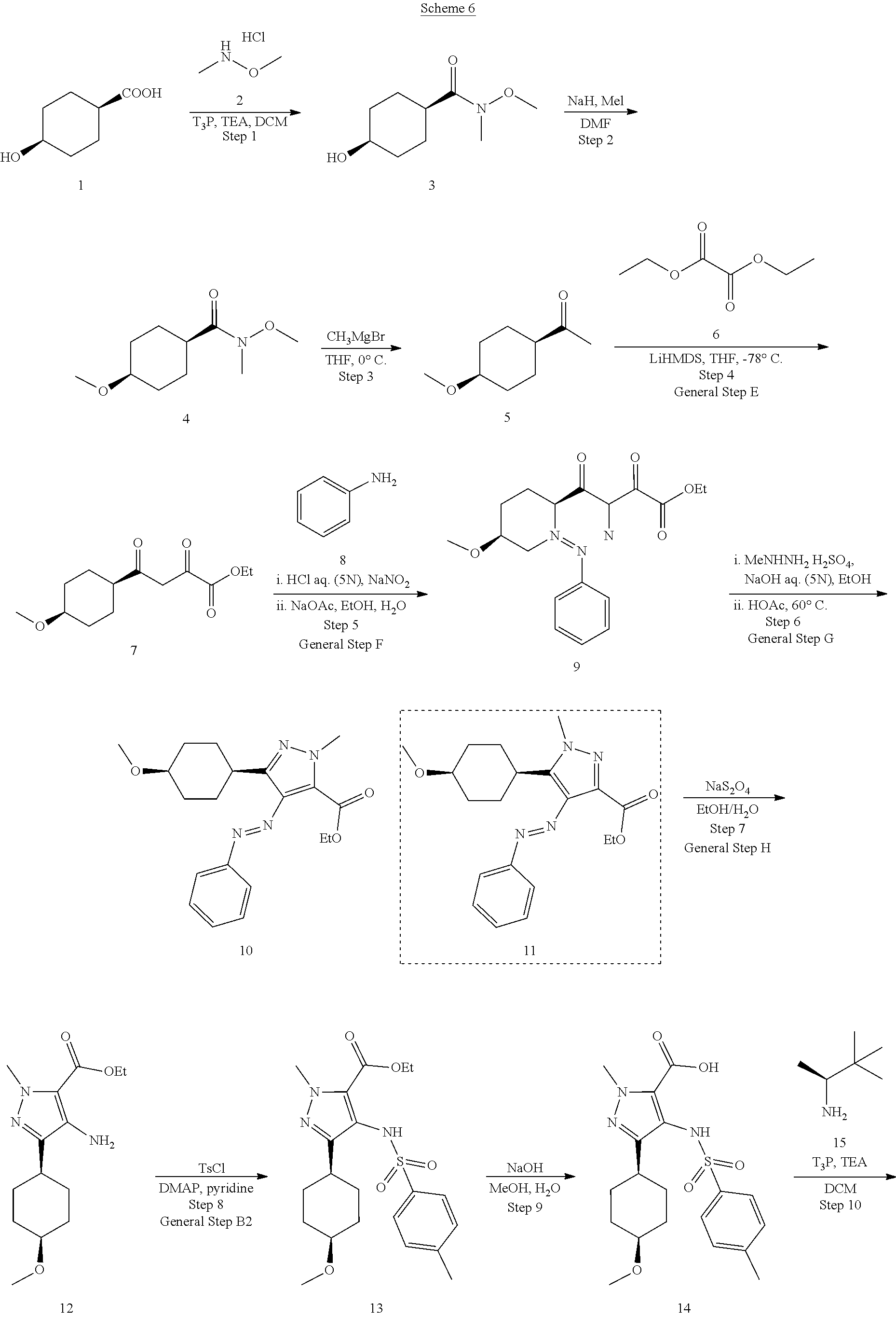

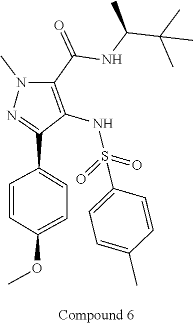

Step 1. Following General Step D, (1s,4s)-4-hydroxy-N-methoxy-N-methylcyclohexane-1-carboxamide was prepared as yellow oil (9.2 g, 71%). Mass (m/z): 188.1 [M+H]⁺.

Step 2. Preparation of (1s,4s)-N,4-dimethoxy-N-methylcyclohexane-1-carboxamide: to a solution of (1s,4s)-4-hydroxy-N-methoxy-N-methylcyclohexane-1-carboxamide (9.2 g, 49 mmol) in DMF (100 mL) was added NaH (60% dispersion in mineral oil, 2.95 g, 73 mmol) at 0° C. The resulting mixture was stirred at ambient temperature under N₂ for 30 minutes before the addition of Mel (10.5 g, 73 mmol). The reaction mixture was further stirred at ambient temperature under N₂ for 16 hrs, and then diluted with water (200 mL). The aqueous solution was extracted with EA (150 mL×3). The combined organic layers were washed with brine (100 mL×3), dried over Na₂SO₄, and filtered. The filtrate was concentrated to dryness to give the product (1s,4s)-N,4-dimethoxy-N-methylcyclohexane-1-carboxamide as brown solid (4.1 g, 40%). Mass (m/z): 202.0 [M+H]⁺.

Step 3. Preparation of 1-((1s,4s)-4-methoxycyclohexyl)ethan-1-one: to a solution of (1s,4s)-N,4-dimethoxy-N-methylcyclohexane-1-carboxamide (3.1 g, 15 mmol) in THF (20 mL) was added MeMgBr (7.7 mL, 3 N solution in 2-Methyl THF, 23 mmol) dropwise at 0° C. under N₂. The resulting solution was stirred at ambient temperature under N₂ for 3 hrs, and then diluted with saturated NH₄Cl aqueous solution (30 mL). The aqueous media was extracted with EA (20 mL×3). The combined organic layers were washed with brine (20 mL×2), dried over Na₂SO₄, and filtered. The filtrate was concentrated under vacuum. The residue was purified by flash column chromatography (PE/EA=5:1) to give the product 1-((1s,4s)-4-methoxycyclohexyl)ethan-1-one as yellow oil (2.4 g, 100%). Mass (m/z): 157.1 [M+H]⁺.

Step 4. (General Step E) Preparation of ethyl 4-((1s,4s)-4-methoxycyclohexyl)-2,4-dioxobutanoate: to a solution of 1-((1s,4s)-4-methoxycyclohexyl)ethan-1-one (2.41 g, 15.4 mmol) and diethyl oxalate (2.25 g, 15.4 mmol) in THF (30 mL) was added LiHMDS (15.4 mL, 1 N solution in THF, 15.4 mmol) dropwise at −78° C. under N₂. The resulting solution was stirred at −78° C. under N₂ for 1 hour, and then diluted with water (40 mL). The aqueous solution was extracted with EA (30 mL×3). The combined organic layers were washed with brine (20 mL×2), dried over Na₂SO₄, and filtered. The filtrate was concentrated to dryness to give the product ethyl 4-((1s,4s)-4-methoxycyclohexyl)-2,4-dioxobutanoate as yellow oil (3.9 g, 100%). Mass (m/z): 257.1 [M+H]⁺.

Step 5. (General Step F) Preparation of rac-ethyl 4-((1s,4s)-4-methoxycyclohexyl)-2,4-dioxo-3-((E)-phenyldiazenyl)butanoate: to a stirred solution of aniline (4.91 g, 52.8 mmol) in 5 N hydrochloric acid aqueous solution (10 mL) was added an ice-cooled solution of sodium nitrite (3.64 g, 52.8 mmol) in water (10 ML) dropwise at 0° C. The resulting solution was stirred at 0° C. for 1 h, and then added dropwise into an ice-cooled suspension of ethyl 4-((1s,4s)-4-methoxycyclohexyl)-2,4-dioxobutanoate (4.5 g, 18 mmol) and sodium acetate (2.89 g, 35.2 mmol) in the mixed solvent of ethanol and water (30 mL, 1:1 (v/v)). The mixture was further stirred at 0-5° C. for 16 hrs, and then diluted with water (50 mL). The aqueous solution was extracted with EA (50 mL×3). The combined organic layers were washed with brine (50 mL×2), dried over Na₂SO₄, and filtered. The filtrate was concentrated to dryness to give the product rac-ethyl 4-((1s,4s)-4-methoxycyclohexyl)-2,4-dioxo-3-((E)-phenyldiazenyl)butanoate as yellow solid (3.7 g, 58%). Mass (m/z): 361.0 [M+H]⁺.

Step 6. (General Step G) Preparation of ethyl 3-((1s,4s)-4-methoxycyclohexyl)-1-methyl-4-((E)-phenyldiazenyl)-1H-pyrazole-5-carboxylate: a solution of methylhydrazine sulfate (1.19 g, 8.25 mmol) in EtOH (5 mL) was basified to pH 8-9 with 5 N NaOH aqueous solution, and then added into a solution of rac-ethyl 4-((1s,4s)-4-methoxycyclohexyl)-2,4-dioxo-3-((E)-phenyldiazenyl)butanoate (2.0 g, 5.5 mmol) in HOAc (15 mL). The resulting solution was stirred at 60° C. for 3 hrs, and then concentrated under vacuum. The residue was purified by flash column chromatography (PE/EA=5:1 to 2:1) to give the product ethyl 3-((1s,4s)-4-methoxycyclohexyl)-1-methyl-4-((E)-phenyldiazenyl)-1H-pyrazole-5-carboxylate as brown solid (600 mg, 29%) and its regio-isomer ethyl 5-((1s,4s)-4-methoxycyclohexyl)-1-methyl-4-((E)-phenyldiazenyl)-1H-pyrazole-3-carboxylate as brown solid (750 mg, 37%).

ethyl 3-((1s,4s)-4-methoxycyclohexyl)-1-methyl-4-((E)-phenyldiazenyl)-1H-pyrazole-5-carboxylate: Mass (m/z): 371.1 [M+H]⁺, Rt=1.489 min (2.0 min);

ethyl 5-((1s,4s)-4-methoxycyclohexyl)-1-methyl-4-((E)-phenyldiazenyl)-1H-pyrazole-3-carboxylate: Mass (m/z): 371.1 [M+H]⁺, Rt=1.369 min (2.0 min)

Step 7. (General Step H) Preparation of ethyl 4-amino-3-((1s,4s)-4-methoxycyclohexyl)-1-methyl-1H-pyrazole-5-carboxylate: to a solution of ethyl 3-((1s,4s)-4-methoxycyclohexyl)-1-methyl-4-((E)-phenyldiazenyl)-1H-pyrazole-5-carboxylate (600 mg, 1.6 mmol) in the mixed solvent of EtOH (16 mL) and water (2 mL) was added Na₂S₂O₄ (2.8 g, 16 mmol). The resulting solution was stirred at 100° C. for 16 hrs, and the organic solvent was then removed under vacuum. The aqueous solution was diluted with water (30 mL), and then extracted with EA (30 mL×3). The combined organic layers were washed with brine (20 mL×2), dried over Na₂SO₄, and filtered. The filtrate was concentrated under vacuum. The residue was purified by flash column chromatography (PE/EA=2:1) to give the product ethyl 4-amino-3-((1s,4s)-4-methoxycyclohexyl)-1-methyl-1H-pyrazole-5-carboxylate as brown solid (170 mg, 37%). Mass (m/z): 282.2 [M+H]⁺.

Step 8. (General Step B2) Preparation of ethyl 3-((1s,4s)-4-methoxycyclohexyl)-1-methyl-4-((4-methylphenyl)sulfonamido)-1H-pyrazole-5-carboxylate: a solution of ethyl 4-amino-3-((1s,4s)-4-methoxycyclohexyl)-1-methyl-1H-pyrazole-5-carboxylate (170 mg, 0.60 mmol), TsCl (172 mg, 0.90 mmol) and DMAP (74 mg, 0.60 mmol) in pyridine (10 mL) was stirred at ambient temperature for 3 hrs, and then filtered. The precipitate was rinsed with 10% EA/PE, and dried to give the product ethyl 3-((1s,4s)-4-methoxycyclohexyl)-1-methyl-4-((4-methylphenyl)sulfonamido)-1H-pyrazole-5-carboxylate as brown solid (330 mg, 60% purity, 76%), which was used in next step without further purification. Mass (m/z): 436.0 [M+H]⁺.

Step 9. Following General Step C, 3-((1s,4s)-4-methoxycyclohexyl)-1-methyl-4-((4-methylphenyl)sulfonamido)-1H-pyrazole-5-carboxylic acid was prepared as brown solid (150 mg, 64%). Mass (m/z): 408.1 [M+H]⁺.

Step 10. Following General Step D, N—((S)-3,3-dimethylbutan-2-yl)-3-((1s,4R)-4-methoxycyclohexyl)-1-methyl-4-((4-methylphenyl)sulfonamido)-1H-pyrazole-5-carboxamide (Compound 6) was prepared as yellow solid (100 mg, 55%). Mass (m/z): 491.2 [M+H]⁺.

### Compound 7: N—((S)-3,3-dimethylbutan-2-yl)-5-((1s,4R)-4-methoxycyclohexyl)-4-((4-methylphenyl)sulfonamido)-1H-pyrazole-3-carboxamide

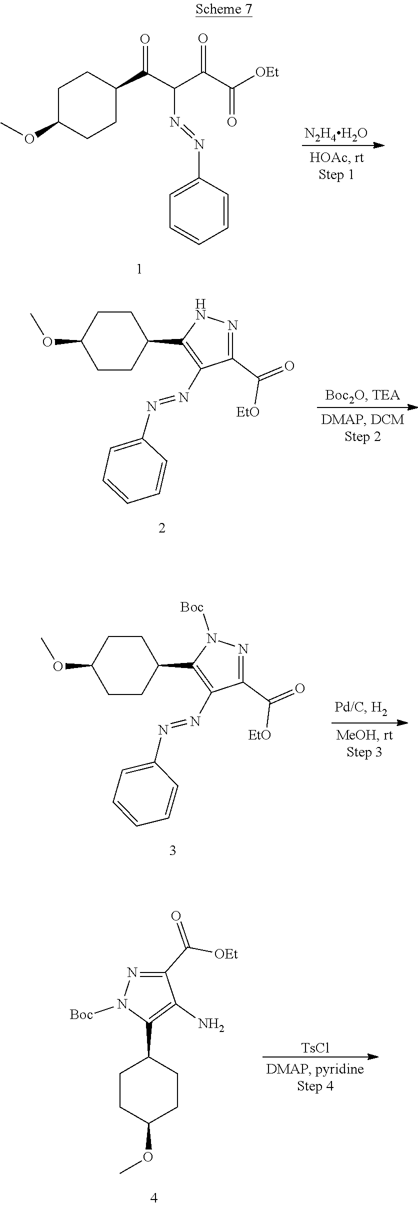

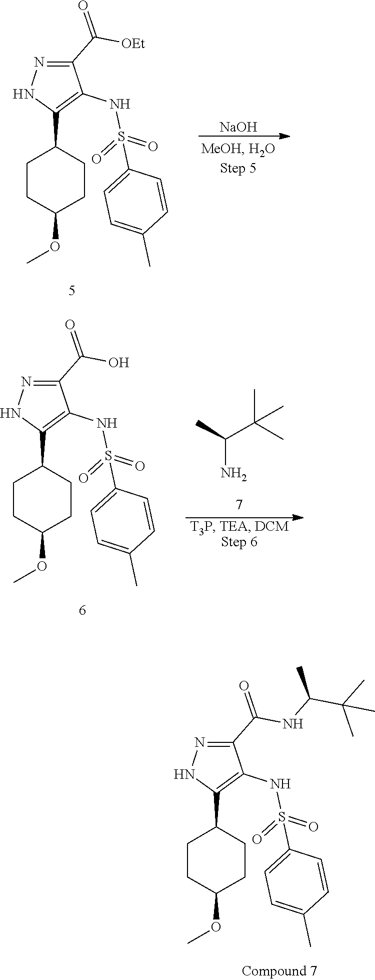

Step 1. Following General Step G, ethyl 5-((1s,4s)-4-methoxycyclohexyl)-4-((E)-phenyldiazenyl)-1H-pyrazole-3-carboxylate was prepared as brown solid (700 mg, 60% purity, 86%), which was used in next step without further purification. Mass (m/z): 357.1 [M+H]⁺.

Step 2. Preparation of 1-(tert-butyl) 3-ethyl 5-((1s,4s)-4-methoxycyclohexyl)-4-((E)-phenyldiazenyl)-1H-pyrazole-1,3-dicarboxylate: to a solution of ethyl 5-((1s,4s)-4-methoxycyclohexyl)-4-((E)-phenyldiazenyl)-1H-pyrazole-3-carboxylate (650 mg, 60% purity, 1.1 mmol), TEA (368 mg, 3.6 mmol) and DMAP (22 mg, 0.18 mmol) in DCM (15 mL) was added Boc₂O (596 mg, 2.7 mmol). The resulting solution was stirred at ambient temperature for 3 hrs, and then concentrated under vacuum. The residue was purified by flash column chromatography (PE/EA=5:1) to give the product 1-(tert-butyl) 3-ethyl 5-((1s,4s)-4-methoxycyclohexyl)-4-((E)-phenyldiazenyl)-1H-pyrazole-1,3-dicarboxylate as yellow solid (110 mg, 22%). Mass (m/z): 457.1 [M+H]⁺.

Step 3. Preparation of 1-(tert-butyl) 3-ethyl 4-amino-5-((1s,4s)-4-methoxycyclohexyl)-1H-pyrazole-1,3-dicarboxylate: to a solution of 1-(tert-butyl) 3-ethyl 5-((1s,4s)-4-methoxycyclohexyl)-4-((E)-phenyldiazenyl)-1H-pyrazole-1,3-dicarboxylate (700 mg, 1.53 mmol) in MeOH (7 mL) was added 10% Pd/C (140 mg, 20% wt/wt) under N₂. The reaction flask was evacuated under vacuum, and then refilled with H₂ (1 atm). The resulting mixture was stirred under H₂ atmosphere at ambient temperature for 16 hrs, and then filtered through Celite. The filtrate was concentrated under reduced pressure to give the product 1-(tert-butyl) 3-ethyl 4-amino-5-((1s,4s)-4-methoxycyclohexyl)-1H-pyrazole-1,3-dicarboxylate as colorless oil (610 mg, 86%). Mass (m/z): 268.1 [M-C₅H₈O₂+H]⁺.

Step 4. Following General Step B2, ethyl 5-((1s,4s)-4-methoxycyclohexyl)-4-((4-methylphenyl)sulfonamido)-1H-pyrazole-3-carboxylate was prepared as yellow solid (137 mg, 23%). Mass (m/z): 422.0 [M+H]⁺.

Step 5. Following General Step C, 5-((1s,4s)-4-methoxycyclohexyl)-4-((4-methylphenyl)sulfonamido)-1H-pyrazole-3-carboxylic acid was prepared as yellow solid (87 mg, 62%). Mass (m/z): 393.8 [M+H]⁺.

Step 6. Following General Step D, N—((S)-3,3-dimethylbutan-2-yl)-5-((1s,4R)-4-methoxycyclohexyl)-4-((4-methylphenyl)sulfonamido)-1H-pyrazole-3-carboxamide (Compound 7) was prepared as white solid (17 mg, 15%). Mass (m/z): 477.8 [M+H]⁺.

### Compound 8: (S)-4-((4-chlorophenyl)sulfonamido)-N-(3,3-dimethylbutan-2-yl)-1-methyl-5-(tetrahydro-2H-pyran-4-yl)-1H-pyrazole-3-carboxamide

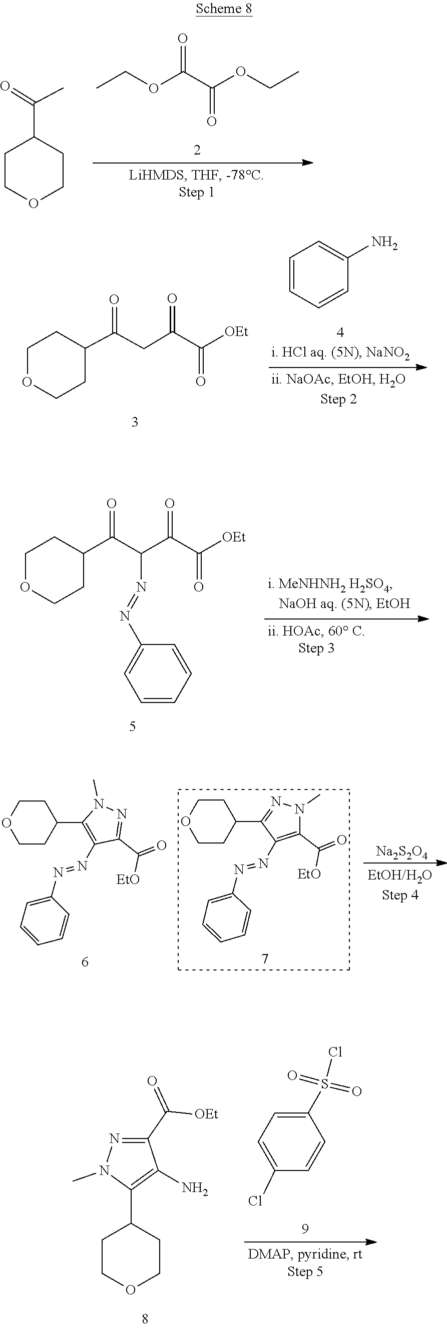

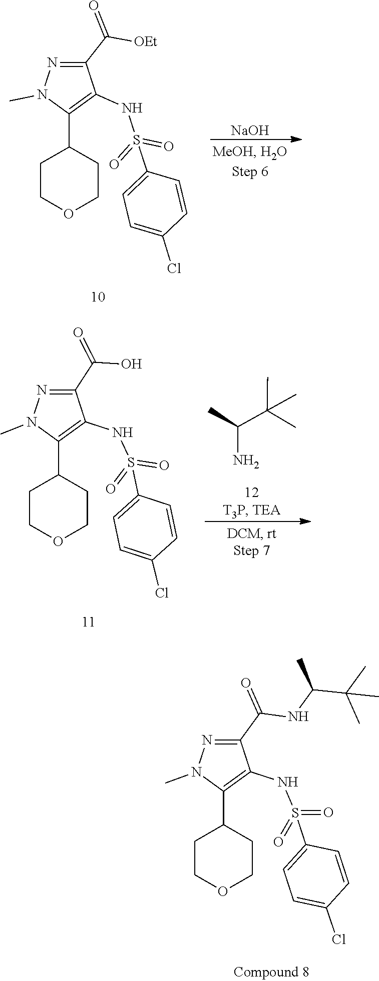

Step 1. Following General Step E, ethyl 2,4-dioxo-4-(tetrahydro-2H-pyran-4-yl)butanoate was prepared as brown oil (900 mg, 50%). Mass (m/z): 229.0 [M+H]⁺.

Step 2. Following General Step F, rac-ethyl (E)-2,4-dioxo-3-(phenyldiazenyl)-4-(tetrahydro-2H-pyran-4-yl)butanoate was prepared as brown solid (1.38 g, 100%). Mass (m/z): 332.8 [M+H]⁺.

Step 3. Following General Step G, ethyl (E)-1-methyl-4-(phenyldiazenyl)-5-(tetrahydro-2H-pyran-4-yl)-1H-pyrazole-3-carboxylate was prepared as brown solid (630 mg, 32%), together with its regio-isomer ethyl (E)-1-methyl-4-(phenyldiazenyl)-3-(tetrahydro-2H-pyran-4-yl)-1H-pyrazole-5-carboxylate as brown solid (350 mg, 17%).

ethyl (E)-1-methyl-4-(phenyldiazenyl)-5-(tetrahydro-2H-pyran-4-yl)-1H-pyrazole-3-carboxylate: Mass (m/z): 342.9 [M+H]⁺, Rt=1.417 min (2.0 min)

ethyl (E)-1-methyl-4-(phenyldiazenyl)-3-(tetrahydro-2H-pyran-4-yl)-1H-pyrazole-5-carboxylate: Mass (m/z): 342.9 [M+H]⁺, Rt=1.530 min (2.0 min)

Step 4. Following General Step H, ethyl 4-amino-1-methyl-5-(tetrahydro-2H-pyran-4-yl)-1H-pyrazole-3-carboxylate was prepared as yellow solid (230 mg, 39%). Mass (m/z): 254.1 [M+H]⁺.

Step 5. Following General Step B2, ethyl 4-((4-chlorophenyl)sulfonamido)-1-methyl-5-(tetrahydro-2H-pyran-4-yl)-1H-pyrazole-3-carboxylate was prepared as brown solid (450 mg, 60% purity, 60%), which was used in next step without further purification. Mass (m/z): 427.7 [M+H]⁺.

Step 6. Following General Step C, 4-((4-chlorophenyl)sulfonamido)-1-methyl-5-(tetrahydro-2H-pyran-4-yl)-1H-pyrazole-3-carboxylic acid was prepared as brown solid (300 mg, 71%). Mass (m/z): 399.7 [M+H]⁺.

Step 7. Following General Step D, (S)-4-((4-chlorophenyl)sulfonamido)-N-(3,3-dimethylbutan-2-yl)-1-methyl-5-(tetrahydro-2H-pyran-4-yl)-1H-pyrazole-3-carboxamide (Compound 8) was prepared as white solid (20 mg, 41%). Mass (m/z): 482.8 [M+H]⁺.

### Compound 9: (S)-4-((4-chlorophenyl)sulfonamido)-N-(3,3-dimethylbutan-2-yl)-1-methyl-3-(tetrahydro-2H-pyran-4-yl)-1H-pyrazole-5-carboxamide

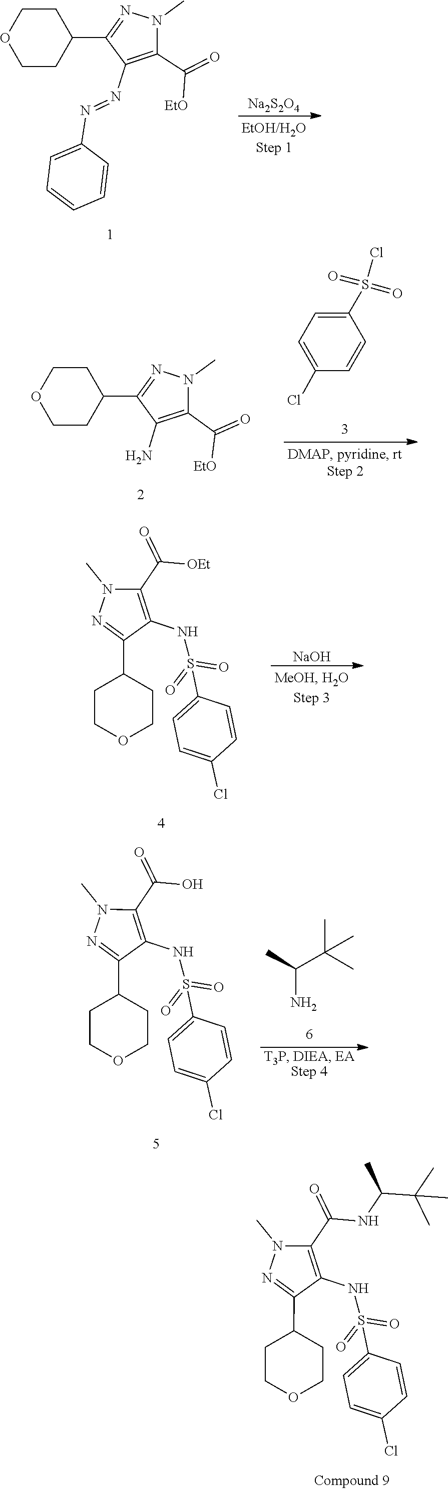

Step 1. Following General Step H, ethyl 4-amino-1-methyl-3-(tetrahydro-2H-pyran-4-yl)-1H-pyrazole-5-carboxylate was prepared as colorless oil (100 mg, 42%). Mass (m/z): 254.1 [M+H]⁺.

Step 2. Following General Step B2, ethyl 4-((4-chlorophenyl)sulfonamido)-1-methyl-3-(tetrahydro-2H-pyran-4-yl)-1H-pyrazole-5-carboxylate was prepared as colorless oil (75 mg, 42%). Mass (m/z): 428.0 [M+H]⁺.

Step 3. Following General Step C, 4-((4-chlorophenyl)sulfonamido)-1-methyl-3-(tetrahydro-2H-pyran-4-yl)-1H-pyrazole-5-carboxylic acid was prepared as colorless solid (80 mg, 82%). Mass (m/z): 400.1 [M+H]⁺.

Step 4. Following General Step D, (S)-4-((4-chlorophenyl)sulfonamido)-N-(3,3-dimethylbutan-2-yl)-1-methyl-3-(tetrahydro-2H-pyran-4-yl)-1H-pyrazole-5-carboxamide (Compound 9) was prepared as white solid (30 mg, 31%). Mass (m/z): 482.7 [M+H]⁺.

### Compound 10: (S)-4-((4-chlorophenyl)sulfonamido)-1-cyclopropyl-N-(3,3-dimethylbutan-2-yl)-3-(tetrahydro-2H-pyran-4-yl)-1H-pyrazole-5-carboxamide

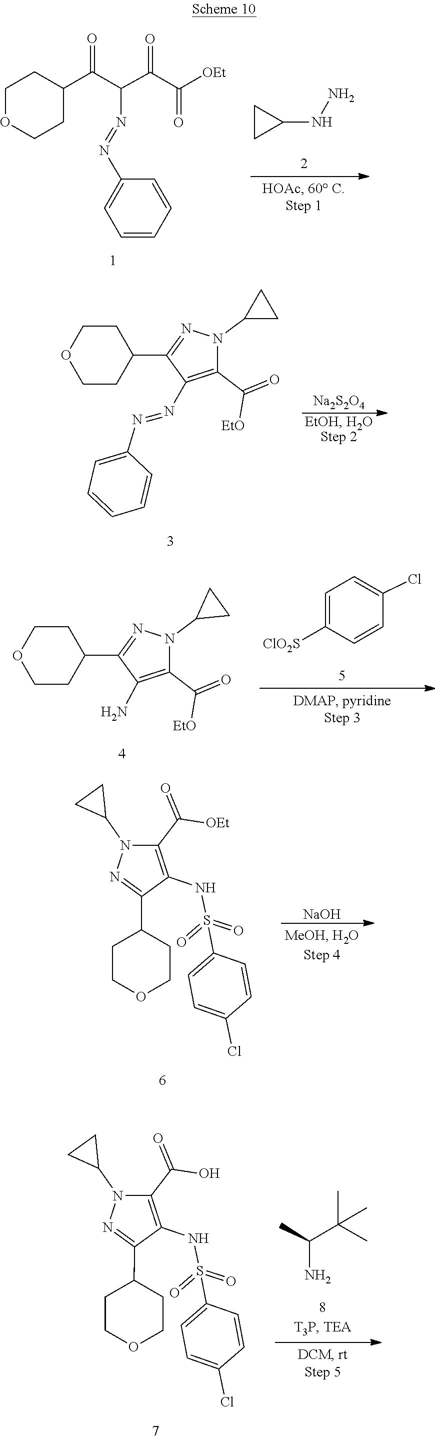

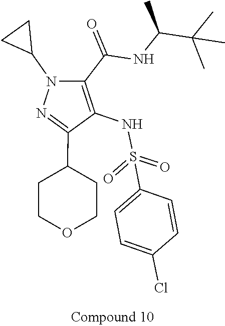 [SMILES: C[C@H](NC(=O)c1c(NS(=O)(=O)c2ccc(Cl)cc2)c(C2CCOCC2)nn1C1CC1)C(C)(C)C]

Step 1. Following General Step G, ethyl (E)-1-cyclopropyl-4-(phenyldiazenyl)-3-(tetrahydro-2H-pyran-4-yl)-1H-pyrazole-5-carboxylate was prepared as yellow oil (600 mg, 23%). Mass (m/z): 368.9 [M+H]⁺.

Step 2. Following General Step H, ethyl 4-amino-1-cyclopropyl-3-(tetrahydro-2H-pyran-4-yl)-1H-pyrazole-5-carboxylate was prepared as yellow solid (220 mg, 42%). Mass (m/z): 280.0 [M+H]⁺.

Step 3. Following General Step B2, ethyl 4-((4-chlorophenyl)sulfonamido)-1-cyclopropyl-3-(tetrahydro-2H-pyran-4-yl)-1H-pyrazole-5-carboxylate was prepared as yellow solid (200 mg, 58%). Mass (m/z): 453.7 [M+H]⁺.

Step 4. Following General Step C, 4-((4-chlorophenyl)sulfonamido)-1-cyclopropyl-3-(tetrahydro-2H-pyran-4-yl)-1H-pyrazole-5-carboxylic acid was prepared as yellow oil (200 mg, 100%). Mass (m/z): 425.6 [M+H]⁺.

Step 5. Following General Step D, (S)-4-((4-chlorophenyl)sulfonamido)-1-cyclopropyl-N-(3,3-dimethylbutan-2-yl)-3-(tetrahydro-2H-pyran-4-yl)-1H-pyrazole-5-carboxamide (Compound 10) was prepared as white solid (97 mg, 31%). Mass (m/z): 508.7 [M+H]⁺.

### Compound 11: (S)-4-((4-chlorophenyl)sulfonamido)-N-(3,3-dimethylbutan-2-yl)-5-(tetrahydro-2H-pyran-4-yl)isoxazole-3-carboxamide

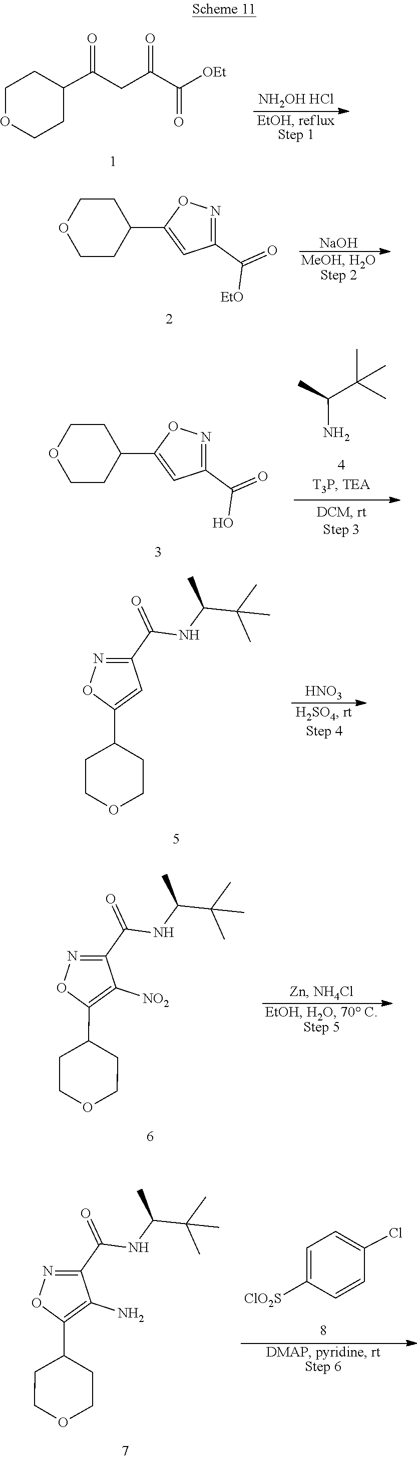

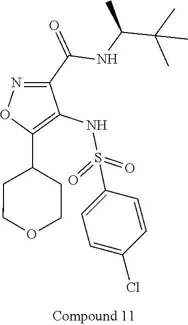 [SMILES: C[C@H](NC(=O)c1noc(C2CCOCC2)c1NS(=O)(=O)c1ccc(Cl)cc1)C(C)(C)C]

Step 1. Preparation of ethyl 5-(tetrahydro-2H-pyran-4-yl)isoxazole-3-carboxylate: a solution of ethyl 2,4-dioxo-4-(tetrahydro-2H-pyran-4-yl)butanoate (500 mg, 2.2 mmol) and hydroxylamine hydrochloride (181 mg, 2.6 mmol) in EtOH (15 mL) was stirred at 90° C. under N₂ for 5 hrs, and then concentrated under vacuum. The residue was partitioned between water (20 mL) and EA (30 mL), and the aqueous media was further extracted with EA (30 mL×2). The combined organic layers were washed with brine (20 mL×3), dried over Na₂SO₄, and then filtered. The filtrate was concentrated to dryness to give the product ethyl 5-(tetrahydro-2H-pyran-4-yl)isoxazole-3-carboxylate as brown oil (425 mg, 86%). Mass (m/z): 226.0 [M+H]⁺.

Step 2. Following General Step C, 5-(tetrahydro-2H-pyran-4-yl)isoxazole-3-carboxylic acid was prepared as yellow solid (160 mg, 81%). Mass (m/z): 219.9 [M+Na]⁺.

Step 3. Following General Step D, (S)—N-(3,3-dimethylbutan-2-yl)-5-(tetrahydro-2H-pyran-4-yl)isoxazole-3-carboxamide was prepared as yellow solid (145 mg, 91%). Mass (m/z): 281.0 [M+H]⁺.

Step 4. Preparation of (S)—N-(3,3-dimethylbutan-2-yl)-4-nitro-5-(tetrahydro-2H-pyran-4-yl)isoxazole-3-carboxamide: to a solution of (S)—N-(3,3-dimethylbutan-2-yl)-5-(tetrahydro-2H-pyran-4-yl)isoxazole-3-carboxamide (145 mg, 0.52 mmol) in concentrated H₂SO₄ (6 mL) was added 100% HNO₃ (2 mL). The resulting solution was stirred at ambient temperature for 16 hrs, and then diluted with ice-cooled water (20 mL) slowly. The aqueous solution was extracted with EA (20 mL×3). The combined organic layers were washed with brine (20 mL×2), dried over Na₂SO₄, and then filtered. The filtrate was concentrated under vacuum to give the product (S)—N-(3,3-dimethylbutan-2-yl)-4-nitro-5-(tetrahydro-2H-pyran-4-yl)isoxazole-3-carboxamide as yellow solid (120 mg, 71%). Mass (m/z): 325.9 [M+H]⁺.

Step 5. Preparation of (S)-4-amino-N-(3,3-dimethylbutan-2-yl)-5-(tetrahydro-2H-pyran-4-yl)isoxazole-3-carboxamide: to a solution of (S)—N-(3,3-dimethylbutan-2-yl)-4-nitro-5-(tetrahydro-2H-pyran-4-yl)isoxazole-3-carboxamide (50 mg, 0.15 mmol) and NH₄Cl (41 mg, 0.77 mmol) in the mixed solvent of EtOH (4 mL) and water (1 mL) was added Zn powder (50 mg, 0.77 mmol). The resulting mixture was stirred at 70° C. for 3 hrs, and then filtered. The filtrate was concentrated under vacuum. The residue was partitioned between water (15 mL) and EA (10 mL), and the aqueous media was further extracted with EA (10 mL×2). The combined organic layers were washed with brine (10 mL×2), dried over Na₂SO₄, and then filtered. The filtrate was concentrated to dryness to give the product (S)-4-amino-N-(3,3-dimethylbutan-2-yl)-5-(tetrahydro-2H-pyran-4-yl)isoxazole-3-carboxamide as yellow solid (33 mg, 72%). Mass (m/z): 296.0 [M+H]⁺.

Step 6. Following General Step B2, (S)-4-((4-chlorophenyl)sulfonamido)-N-(3,3-dimethylbutan-2-yl)-5-(tetrahydro-2H-pyran-4-yl)isoxazole-3-carboxamide (Example 11) was prepared as white solid (14 mg, 26%). Mass (m/z): 469.9 [M+H]⁺.

### Compound 12: (S)-4-((4-cyclopropylphenyl)sulfonamido)-N-(3,3-dimethylbutan-2-yl)-5-(tetrahydro-2H-pyran-4-yl)isoxazole-3-carboxamide

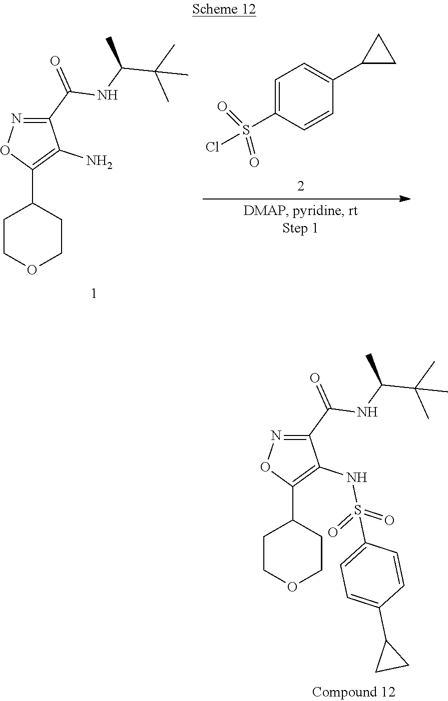 [SMILES: C[C@H](NC(=O)c1noc(C2CCOCC2)c1N)C(C)(C)C ; O=S(=O)(Cl)c1ccc(C2CC2)cc1 ; C[C@H](NC(=O)c1noc(C2CCOCC2)c1NS(=O)(=O)c1ccc(C2CC2)cc1)C(C)(C)C]

Step 1. Following General Step B2, (S)-4-((4-cyclopropylphenyl)sulfonamido)-N-(3,3-dimethylbutan-2-yl)-5-(tetrahydro-2H-pyran-4-yl)isoxazole-3-carboxamide (Compound 12) was prepared as white solid (16 mg, 9%). Mass (m/z): 475.8 [M+H]⁺.

### Compound 13: N—((S)-3,3-dimethylbutan-2-yl)-5-((1s,4R)-4-methoxycyclohexyl)-1-methyl-4-((4-methylphenyl)sulfonamido)-1H-pyrazole-3-carboxamide

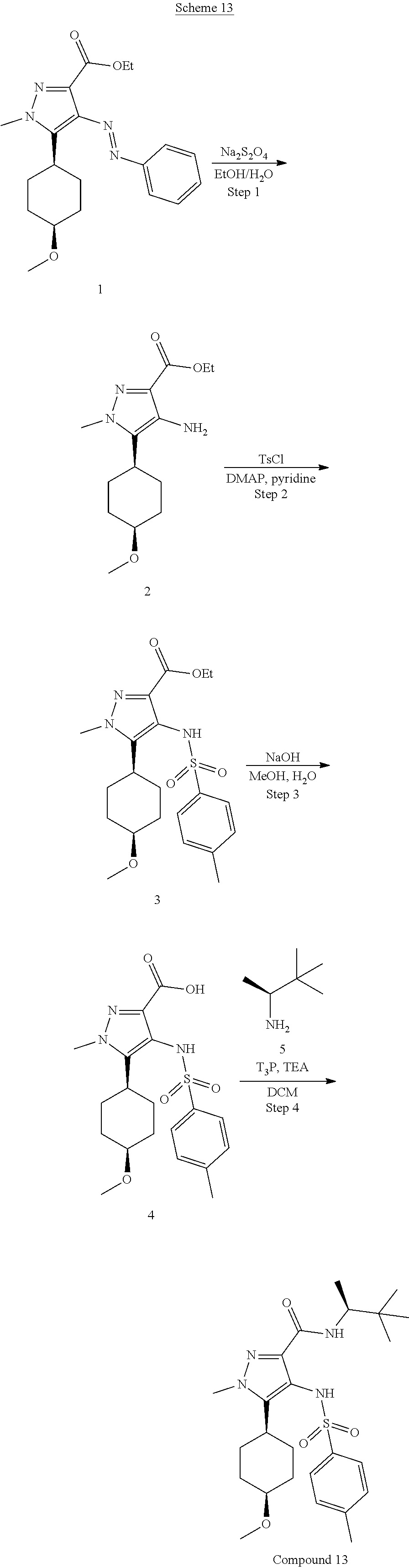

Step 1. Following General Step H, ethyl 4-amino-5-((1s,4s)-4-methoxycyclohexyl)-1-methyl-1H-pyrazole-3-carboxylate was prepared as yellow solid (290 mg, 45%). Mass (m/z): 282.1 [M+H]⁺.

Step 2. Following General Step B2, ethyl 5-((1s,4s)-4-methoxycyclohexyl)-1-methyl-4-((4-methylphenyl)sulfonamido)-1H-pyrazole-3-carboxylate was prepared as brown solid (330 mg, 60% purity, 62%). Mass (m/z): 436.0 [M+H]⁺.

Step 3. Following General Step C, 5-((1s,4s)-4-methoxycyclohexyl)-1-methyl-4-((4-methylphenyl)sulfonamido)-1H-pyrazole-3-carboxylic acid was prepared as brown solid (150 mg, 64%). Mass (m/z): 408.0 [M+H]⁺.

Step 4. Following General Step D, N—((S)-3,3-dimethylbutan-2-yl)-5-((1s,4R)-4-methoxycyclohexyl)-1-methyl-4-((4-methylphenyl)sulfonamido)-1H-pyrazole-3-carboxamide (Compound 13) was prepared as yellow solid (200 mg, 52%). Mass (m/z): 491.1 [M+H]⁺.

### Compound 14: (S)—N-(3,3-dimethylbutan-2-yl)-3-(4-(methoxymethyl)phenyl)-1-methyl-4-((4-methylphenyl)sulfonamido)-1H-pyrazole-5-carboxamide

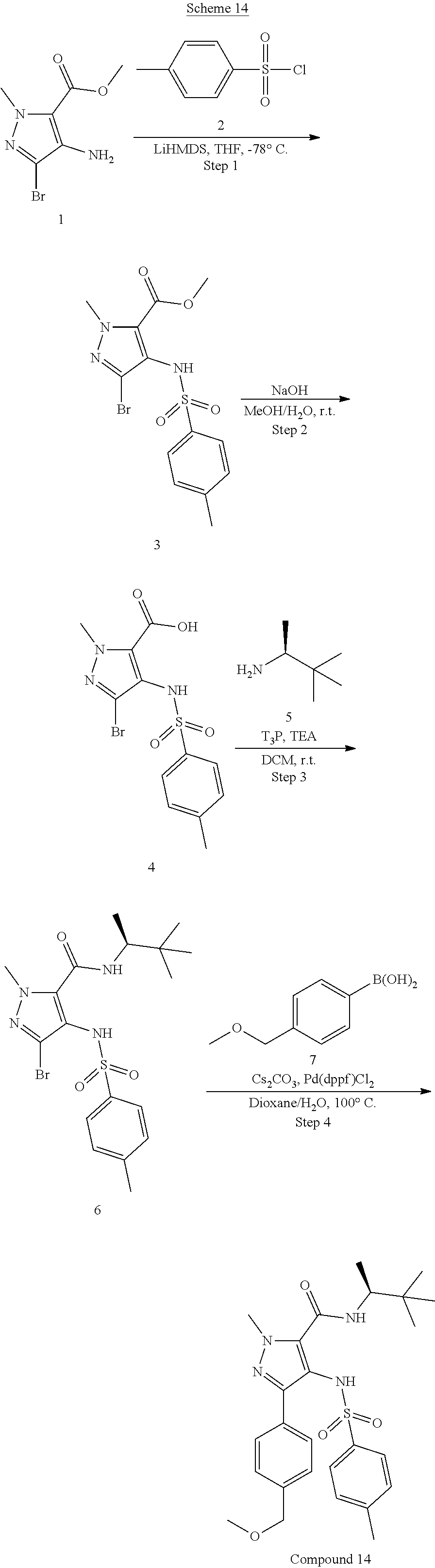

Step 1. Following General Step B1, methyl 5-bromo-2-methyl-4-[(4-methylbenzene)sulfonamido]pyrazole-3-carboxylate was prepared as yellow solid (2.6 g, 80%). Mass (m/z): 387.8 [M+H]⁺.

Step 2. Following General Step C, 5-bromo-2-methyl-4-[(4-methylbenzene)sulfonamido]pyrazole-3-carboxylic acid was prepared as yellow solid (2.36 g, 90%). Mass (m/z): 373.8 [M+H]⁺.

Step 3. Following General Step D, 5-bromo-4-[(4-methylbenzene)sulfonamido]-N-[(2S)-3,3-dimethylbutan-2-yl]-2-methylpyrazole-3-carboxamide was prepared as yellow solid (1.3 g, 50%). Mass (m/z): 456.7 [M+H]⁺.

Step 4. Following General Step A, (S)—N-(3,3-dimethylbutan-2-yl)-3-(4-(methoxymethyl)phenyl)-1-methyl-4-((4-methylphenyl)sulfonamido)-1H-pyrazole-5-carboxamide (Compound 14) was prepared as white solid (22 mg, 25%). Mass (m/z): 499.0 [M+H]⁺.

### Compound 15: (S)—N-(3,3-dimethylbutan-2-yl)-3-(3-(methoxymethyl)phenyl)-1-methyl-4-((4-methylphenyl)sulfonamido)-1H-pyrazole-5-carboxamide

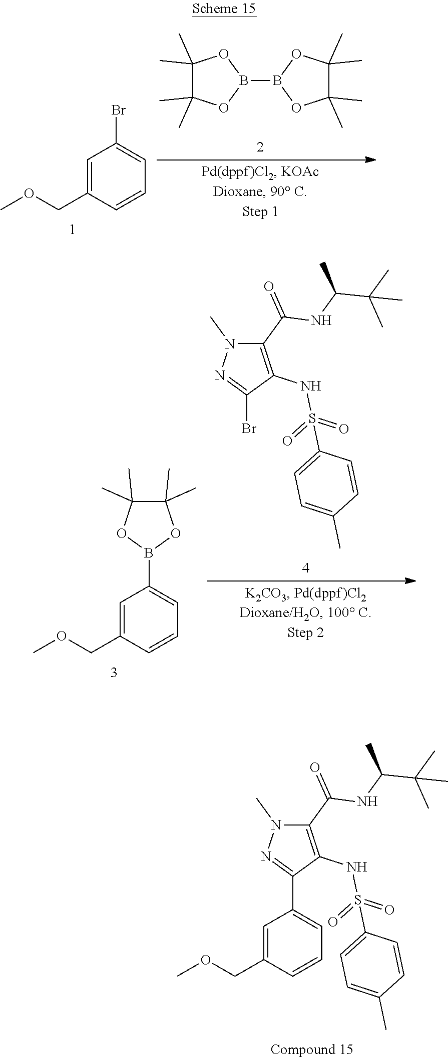

Step 1. Preparation of 2-(3-(methoxymethyl)phenyl)-4,4,5,5-tetramethyl-1,3,2-dioxaborolane: to a solution of 1-bromo-3-(methoxymethyl)benzene (500 mg, 2.5 mmol) in dioxane (20 mL) under N₂ were added bis(pinacolato)diboron (947 mg, 3.7 mmol), KOAc (1.21 g, 12.4 mmol) and Pd(dppf)Cl₂ (182 mg, 0.25 mmol). The resulting mixture was stirred at 90° C. under N₂ for 16 hrs, and then concentrated under vacuum. The residue was purified by flash column chromatography (PE/EA=10:1) to give the product 2-(3-(methoxymethyl)phenyl)-4,4,5,5-tetramethyl-1,3,2-dioxaborolane as colorless oil (404 mg, 65%). Mass (m/z): 249.0 [M+H]⁺.

Step 2. Following General Step A, (S)—N-(3,3-dimethylbutan-2-yl)-3-(3-(methoxymethyl)phenyl)-1-methyl-4-((4-methylphenyl)sulfonamido)-1H-pyrazole-5-carboxamide (Compound 15) was prepared as white solid (12 mg, 16%). Mass (m/z): 498.8 [M+H]⁺.

### Compound 16: (S)-3-(3,6-dihydro-2H-pyran-4-yl)-N-(3,3-dimethylbutan-2-yl)-1-methyl-4-((4-methylphenyl)sulfonamido)-1H-pyrazole-5-carboxamide

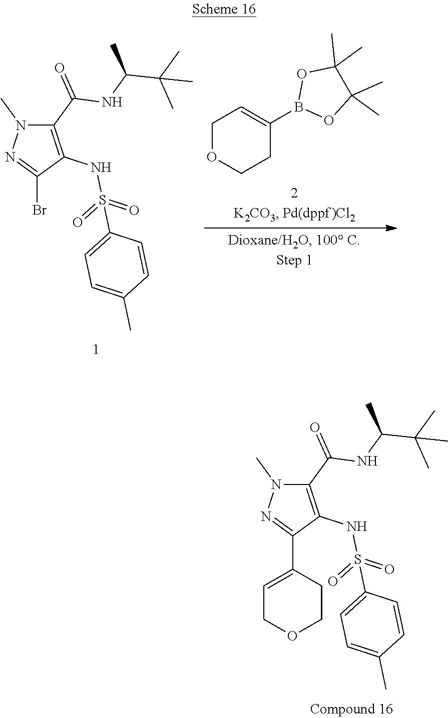

Step 1. Following General Step A, (S)-3-(3,6-dihydro-2H-pyran-4-yl)-N-(3,3-dimethylbutan-2-yl)-1-methyl-4-((4-methylphenyl)sulfonamido)-1H-pyrazole-5-carboxamide (Compound 16) was prepared as yellow solid (15 mg, 21%). Mass (m/z): 460.9 [M+H]⁺.

### Compound 17: tert-butyl (S)-4-(5-((3,3-dimethylbutan-2-yl)carbamoyl)-1-methyl-4-((4-methylphenyl)sulfonamido)-1H-pyrazol-3-yl)-3,6-dihydropyridine-1(2H)-carboxylate

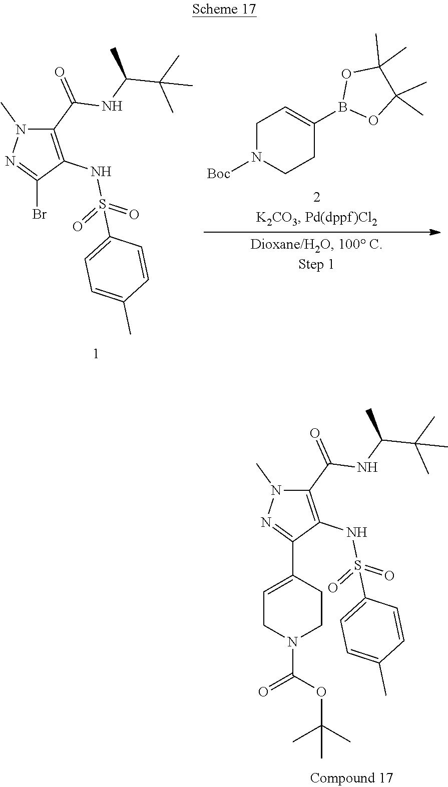 [SMILES: Cc1ccc(S(=O)(=O)Nc2c(Br)nn(C)c2C(=O)N[C@@H](C)C(C)(C)C)cc1 ; CC(C)(C)OC(=O)N1CC=C(B2OC(C)(C)C(C)(C)O2)CC1 ; Cc1ccc(S(=O)(=O)Nc2c(C3=CCN(C(=O)OC(C)(C)C)CC3)nn(C)c2C(=O)N[C@@H](C)C(C)(C)C)cc1]

Step 1. Following General Step A, tert-butyl (S)-4-(5-((3,3-dimethylbutan-2-yl)carbamoyl)-1-methyl-4-((4-methylphenyl)sulfonamido)-1H-pyrazol-3-yl)-3,6-dihydropyridine-1(2H)-carboxylate (Compound 17) was prepared as white solid (27 mg, 31%). Mass (m/z): 581.7 [M+Na]⁺.

### Compound 18: (S)—N-(3,3-dimethylbutan-2-yl)-3-(4,4-dimethylcyclohex-1-en-1-yl)-1-methyl-4-((4-methylphenyl)sulfonamido)-1H-pyrazole-5-carboxamide

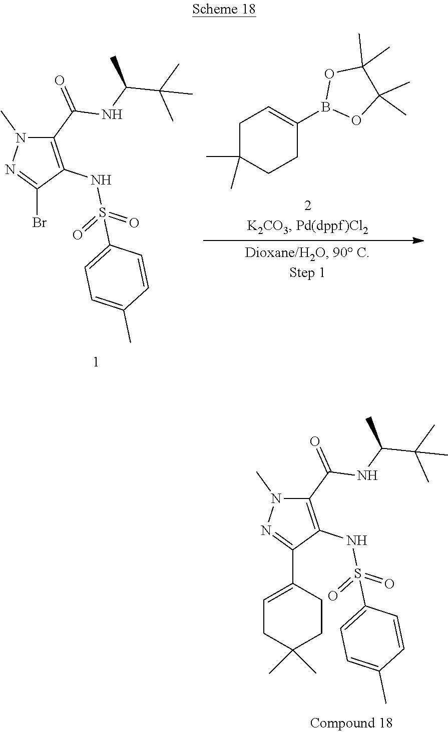

Step 1. Following General Step A, (S)—N-(3,3-dimethylbutan-2-yl)-3-(4,4-dimethylcyclohex-1-en-1-yl)-1-methyl-4-((4-methylphenyl)sulfonamido)-1H-pyrazole-5-carboxamide (Compound 18) was prepared as white solid (20 mg, 31%). Mass (m/z): 486.8 [M+H]⁺.

### Compound 19: 4-((4-chlorophenyl)sulfonamido)-N—((S)-3,3-dimethylbutan-2-yl)-3-((1S,4R)-4-methoxycyclohexyl)-1-methyl-1H-pyrazole-5-carboxamide

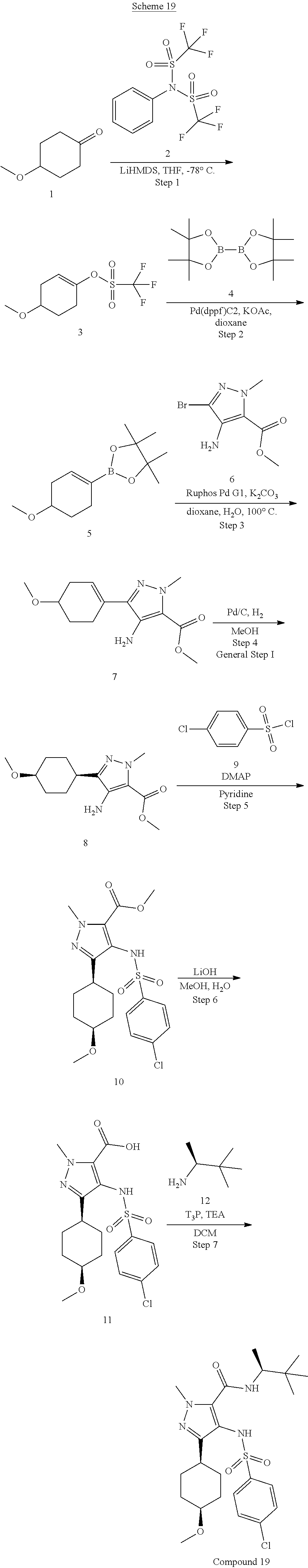

### Step 1. Preparation of 4-methoxycyclohex-1-en-1-yl trifluoromethanesulfonate

To a stirred solution of 4-methoxycyclohexan-1-one (2 g, 15.6 mmol) in THF (20 mL) was added a solution of LiHMDS in THF (23.4 mL, 1 N, 23.4 mmol) at −78° C. The resulting solution was stirred at −78° C. for 30 minutes before the addition of a solution of 1,1,1-trifluoro-N-phenyl-N-(trifluoromethane)sulfonylmethanesulfonamide (6.68 g, 18 mmol) in THF (10 mL). The mixture was further stirred at −78° C. for 1 hour, and then diluted with saturated NH₄Cl aqueous solution (50 mL). The aqueous solution was extracted with EA (30 mL×3). The combined organic layers were washed with brine (30 mL×2), dried over Na₂SO₄, and filtered. The filtrate was concentrated under vacuum. The crude residue was purified by flash column chromatography (PE:EA=5:1) to give the product 4-methoxycyclohex-1-en-1-yl trifluoromethanesulfonate as a brown oil (2.4 g, 60%).

### Step 2. Preparation of 2-(4-methoxycyclohex-1-en-1-yl)-4,4,5,5-tetramethyl-1,3,2-dioxaborolane

To a solution of 4-methoxycyclohex-1-en-1-yl trifluoromethanesulfonate (1 g, 3.8 mmol), 4,4,5,5-tetramethyl-2-(4,4,5,5-tetramethyl-1,3,2-dioxaborolan-2-yl)-1,3,2-dioxaborolane (1.16 g, 4.56 mmol) and KOAc (1.12 g, 11.4 mmol) in dioxane (20 mL) was added Pd(dppf)Cl₂ (140 mg, 0.19 mmol). The reaction mixture was stirred at 100° C. under N₂ for 16 hrs, and then concentrated under vacuum. The crude residue was purified by flash column chromatography (PE/EA=10:1) to give the product 2-(4-methoxycyclohex-1-en-1-yl)-4,4,5,5-tetramethyl-1,3,2-dioxaborolane as a yellow oil (500 mg, 58%).

Step 3. Following General Step A, methyl 4-amino-3-(4-methoxycyclohex-1-en-1-yl)-1-methyl-1H-pyrazole-5-carboxylate was prepared as a yellow solid (260 mg, 99%). Mass (m/z): 266.1 [M+H]⁺.

### Step 4. (General Step I) Preparation of methyl 4-amino-3-((1s,4s)-4-methoxycyclohexyl)-1-methyl-1H-pyrazole-5-carboxylate

A mixture of methyl 4-amino-3-(4-methoxycyclohex-1-en-1-yl)-1-methyl-1H-pyrazole-5-carboxylate (260 mg, 0.98 mmol) and 10% Pd/C (52 mg, 20% wt/wt) in MeOH (10 mL) was stirred at ambient temperature under H₂ atmosphere for 16 hrs. The resulting mixture was filtered through Celite, and the filtrate was concentrated under vacuum. The crude residue was purified by preparative TLC (PE/EA=2:1) to give the product methyl 4-amino-3-((1s,4s)-4-methoxycyclohexyl)-1-methyl-1H-pyrazole-5-carboxylate as a light brown solid (150 mg, 57%). Mass (m/z): 268.1 [M+H]⁺.

Step 5. Following General Step B2, methyl 4-((4-chlorophenyl)sulfonamido)-3-((1S,4S)-4-methoxycyclohexyl)-1-methyl-1H-pyrazole-5-carboxylate was prepared as a light brown solid (230 mg, 92%). Mass (m/z): 442.0 [M+H]⁺.

Step 6. Following General Step C, 4-((4-chlorophenyl)sulfonamido)-3-((1S,4S)-4-methoxycyclohexyl)-1-methyl-1H-pyrazole-5-carboxylic acid was prepared as a yellow solid (180 mg, 80%). Mass (m/z): 427.9 [M+H]⁺.

Step 7. Following General Step D, 4-((4-chlorophenyl)sulfonamido)-N—((S)-3,3-dimethylbutan-2-yl)-3-((1S,4R)-4-methoxycyclohexyl)-1-methyl-1H-pyrazole-5-carboxamide (Compound 19) was prepared as a yellow solid (160 mg, 74%). Mass (m/z): 511.0 [M+H]⁺.

### Compound 20: 4-((4-cyclopropylphenyl)sulfonamido)-N—((S)-3,3-dimethylbutan-2-yl)-3-((1S,4R)-4-methoxycyclohexyl)-1-methyl-1H-pyrazole-5-carboxamide

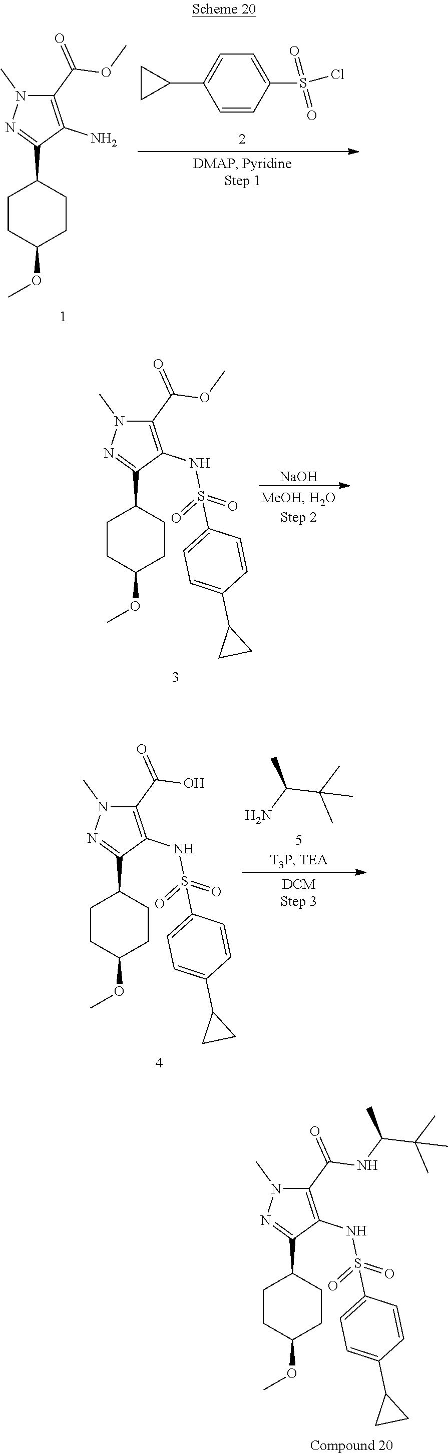

Step 1. Following General Step B2, methyl 4-((4-cyclopropylphenyl)sulfonamido)-3-((1S,4S)-4-methoxycyclohexyl)-1-methyl-1H-pyrazole-5-carboxylate was prepared as a brown solid (140 mg, 83%). Mass (m/z): 447.9 [M+H]⁺.

Step 2. Following General Step C, 4-((4-cyclopropylphenyl)sulfonamido)-3-((1S,4S)-4-methoxycyclohexyl)-1-methyl-1H-pyrazole-5-carboxylic acid was prepared as a white solid (150 mg, 88%). Mass (m/z): 434.1 [M+H]⁺.

Step 3. Following General Step D, 4-((4-cyclopropylphenyl)sulfonamido)-N—((S)-3,3-dimethylbutan-2-yl)-3-((1S,4R)-4-methoxycyclohexyl)-1-methyl-1H-pyrazole-5-carboxamide (Compound 20) was prepared as a white solid (40 mg, 28%). Mass (m/z): 517.1 [M+H]⁺.

### Compound 21: 4-((4-cyanophenyl)sulfonamido)-N—((S)-3,3-dimethylbutan-2-yl)-3-((1S,4R)-4-methoxycyclohexyl)-1-methyl-1H-pyrazole-5-carboxamide

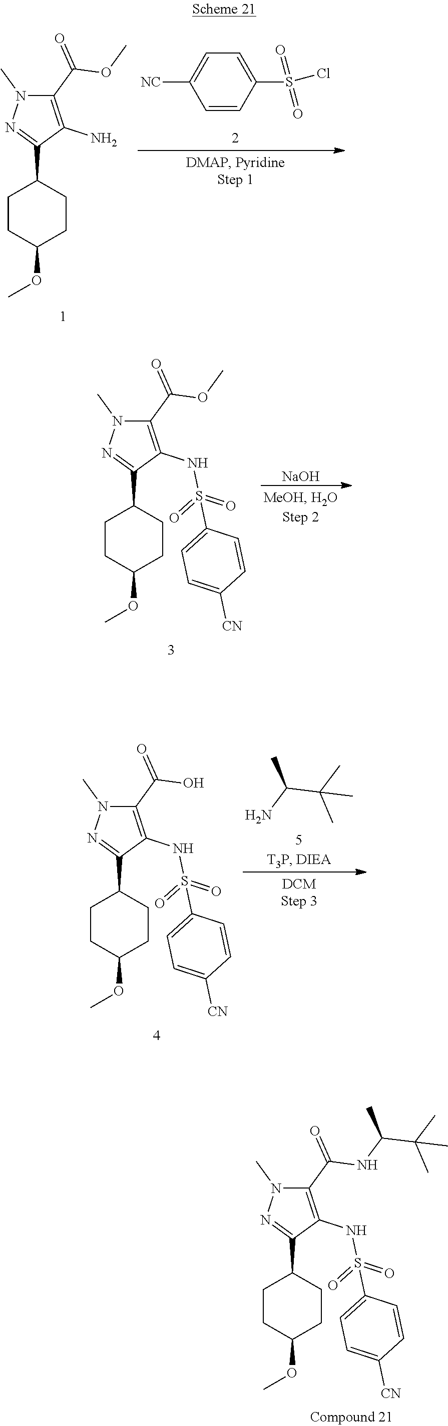

Step 1. Following General Step B2, methyl 4-((4-cyanophenyl)sulfonamido)-3-((1S,4S)-4-methoxycyclohexyl)-1-methyl-1H-pyrazole-5-carboxylate was prepared as a white solid (550 mg, 62%). Mass (m/z): 433.1 [M+H]⁺.

Step 2. Following General Step C, 4-((4-cyanophenyl)sulfonamido)-3-((1S,4S)-4-methoxycyclohexyl)-1-methyl-1H-pyrazole-5-carboxylic acid was prepared as a yellow solid (470 mg, 78%). Mass (m/z): 418.9 [M+H]⁺.

Step 3. Following General Step D, 4-((4-cyanophenyl)sulfonamido)-N—((S)-3,3-dimethylbutan-2-yl)-3-((1s,4R)-4-methoxycyclohexyl)-1-methyl-1H-pyrazole-5-carboxamide (Compound 21) was prepared as a white solid (114 mg, 20%). Mass (m/z): 501.9 [M+H]⁺.

### Compound 22: 4-((4-chlorophenyl)sulfonamido)-N—((S)-3,3-dimethylbutan-2-yl)-3-(4-methoxycyclohex-1-en-1-yl)-1-methyl-1H-pyrazole-5-carboxamide

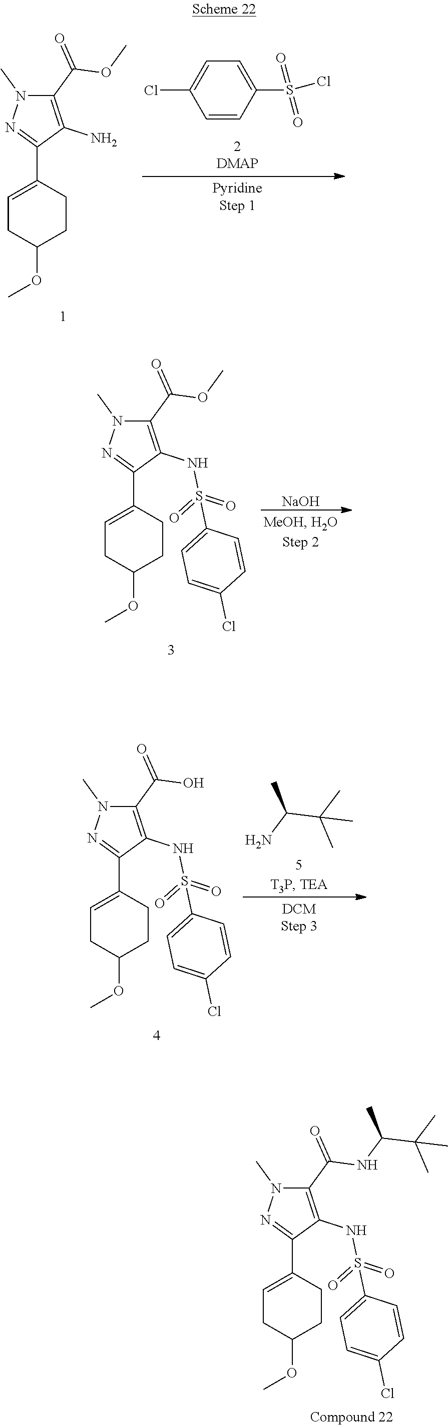

Step 1. Following General Step B2, methyl 4-((4-chlorophenyl)sulfonamido)-3-(4-methoxycyclohex-1-en-1-yl)-1-methyl-1H-pyrazole-5-carboxylate was prepared as a colorless oil (66 mg, 39%). Mass (m z): 462.1 [M+Na]⁺.

Step 2. Following General Step C, 4-((4-chlorophenyl)sulfonamido)-3-(4-methoxycyclohex-1-en-1-yl)-1-methyl-1H-pyrazole-5-carboxylic acid was prepared as a white solid (50 mg, 78%). Mass (m/z): 426.1 [M+H]⁺.

Step 3. Following General Step D, 4-((4-chlorophenyl)sulfonamido)-N—((S)-3,3-dimethylbutan-2-yl)-3-(4-methoxycyclohex-1-en-1-yl)-1-methyl-1H-pyrazole-5-carboxamide (Compound 22) was prepared as a white solid (38 mg, 63%). Mass (m/z): 509.2 [M+H]⁺.

### Compound 23: (S)-4-((4-chlorophenyl)sulfonamido)-N-(3,3-dimethylbutan-2-yl)-1-methyl-3-(1,4-dioxaspiro[4.5]decan-8-yl)-1H-pyrazole-5-carboxamide

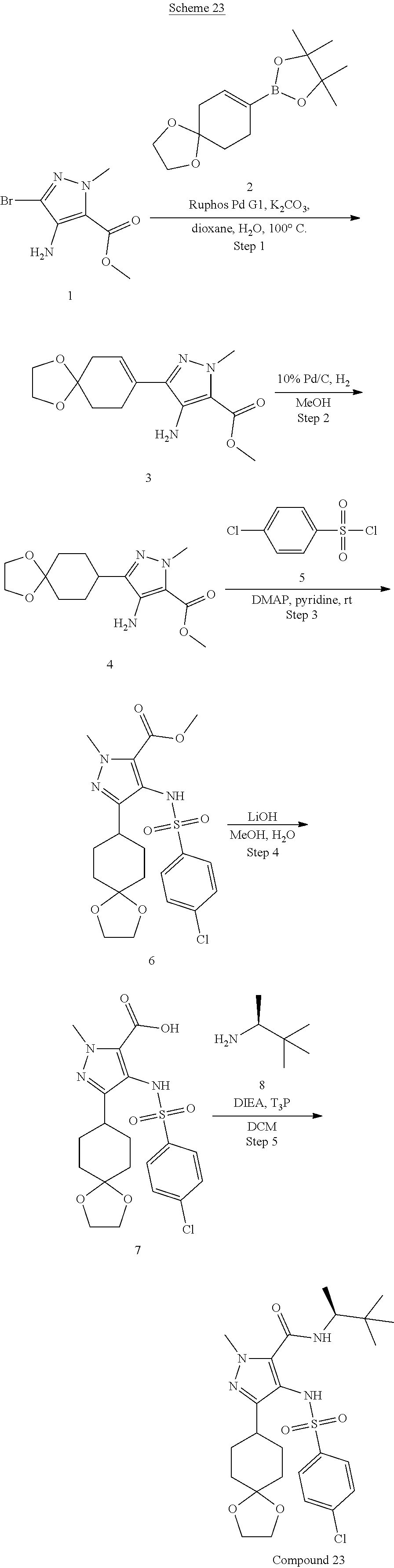

Step 1. Following General Step A, methyl 4-amino-1-methyl-3-(1,4-dioxaspiro[4.5]dec-7-en-8-yl)-1H-pyrazole-5-carboxylate was prepared as a yellow solid (2 g, 80%). Mass (m/z): 294.0 [M+H]⁺.

Step 2. Following General Step I, methyl 4-amino-1-methyl-3-(1,4-dioxaspiro[4.5]decan-8-yl)-1H-pyrazole-5-carboxylate was prepared as a brown solid (2 g, 100%). Mass (m/z): 296.1 [M+H]⁺.

Step 3. Following General Step B2, methyl 4-((4-chlorophenyl)sulfonamido)-1-methyl-3-(1,4-dioxaspiro[4.5]decan-8-yl)-1H-pyrazole-5-carboxylate was prepared as a yellow solid (330 mg, 62%). Mass (m/z): 469.8 [M+H]⁺.

Step 4. Following General Step C, 4-((4-chlorophenyl)sulfonamido)-1-methyl-3-(1,4-dioxaspiro[4.5]decan-8-yl)-1H-pyrazole-5-carboxylic acid was prepared as a white solid (320 mg, 90%). Mass (m/z): 455.8 [M+H]⁺.

Step 5. Following General Step D, (S)-4-((4-chlorophenyl)sulfonamido)-N-(3,3-dimethylbutan-2-yl)-1-methyl-3-(1,4-dioxaspiro[4.5]decan-8-yl)-1H-pyrazole-5-carboxamide (Compound 23) was prepared as a white solid (206 mg, 53%). Mass (m/z): 539.2 [M+H]⁺.

### Compound 24: (S)-4-((4-chlorophenyl)sulfonamido)-N-(3,3-dimethylbutan-2-yl)-1-methyl-3-(1,4-dioxaspiro[4.5]dec-7-en-8-yl)-1H-pyrazole-5-carboxamide

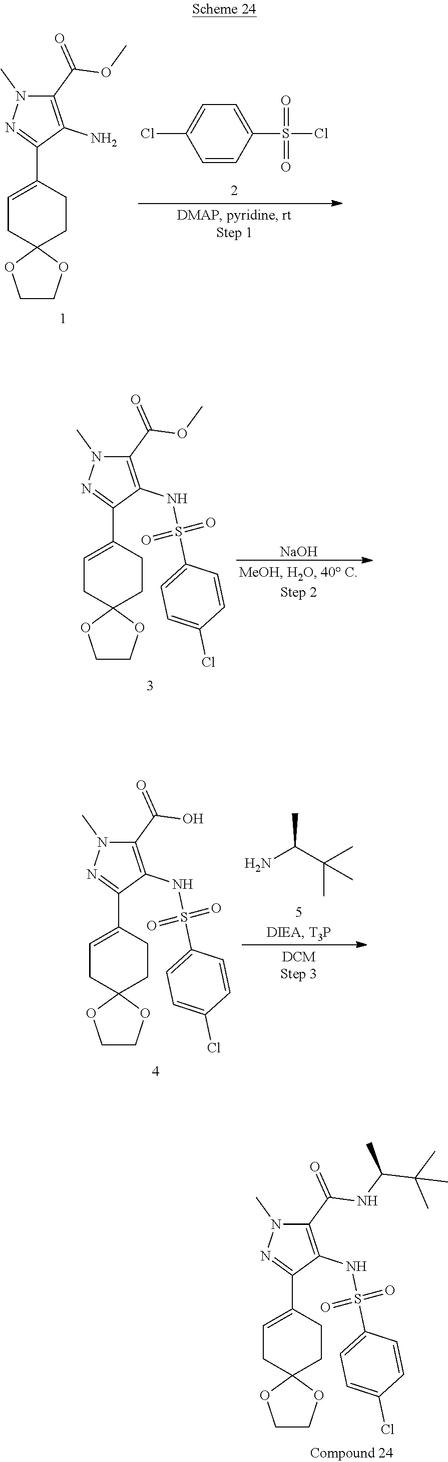

Step 1. Following General Step B2, methyl 4-((4-chlorophenyl)sulfonamido)-1-methyl-3-(1,4-dioxaspiro[4.5]dec-7-en-8-yl)-1H-pyrazole-5-carboxylate was prepared as a yellow oil (415 mg, 65%). Mass (m z): 468.0 [M+H]⁺.

Step 2. Following General Step C, 4-((4-chlorophenyl)sulfonamido)-1-methyl-3-(1,4-dioxaspiro[4.5]dec-7-en-8-yl)-1H-pyrazole-5-carboxylic acid was prepared as a yellow oil (400 mg, 82%). Mass (m/z): 454.1 [M+H]⁺.

Step 3. Following General Step D, (S)-4-((4-chlorophenyl)sulfonamido)-N-(3,3-dimethylbutan-2-yl)-1-methyl-3-(1,4-dioxaspiro[4.5]dec-7-en-8-yl)-1H-pyrazole-5-carboxamide (Compound 24) was prepared as a white solid (225 mg, 47%). Mass (m/z): 537.2 [M+H]⁺.

### Compound 25: (S)-4-((4-cyanophenyl)sulfonamido)-N-(3,3-dimethylbutan-2-yl)-1-methyl-3-(tetrahydro-2H-pyran-4-yl)-1H-pyrazole-5-carboxamide

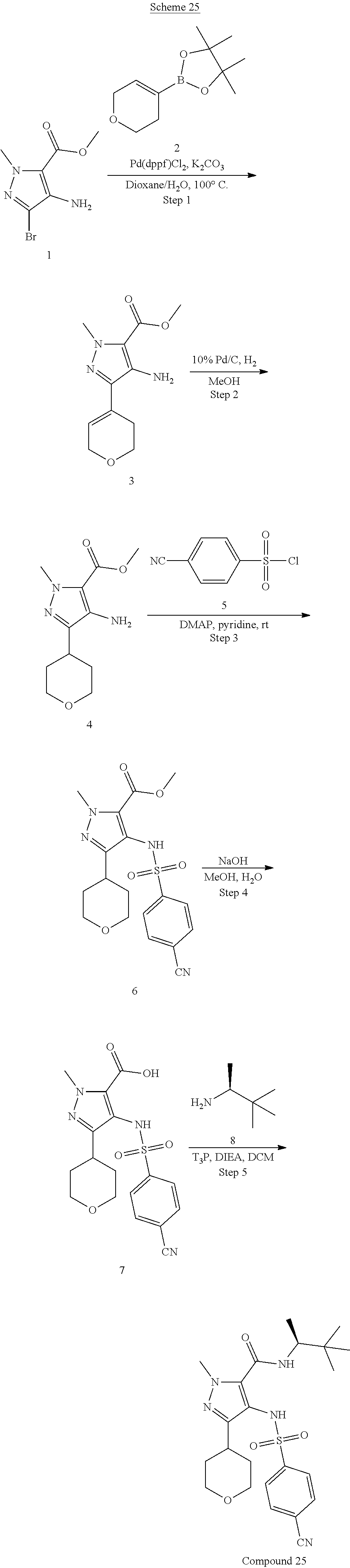

Step 1. Following General Step A, methyl 4-amino-3-(3,6-dihydro-2H-pyran-4-yl)-1-methyl-1H-pyrazole-5-carboxylate was prepared as a yellow solid (1.22 g, 96%). Mass (m/z): 238.1 [M+H]⁺.

Step 2. Following General Step I, methyl 4-amino-1-methyl-3-(tetrahydro-2H-pyran-4-yl)-1H-pyrazole-5-carboxylate was prepared as yellow oil (1.19 g, 96%). Mass (m z): 240.0 [M+H]⁺.

Step 3. Following General Step B2, methyl 4-((4-cyanophenyl)sulfonamido)-1-methyl-3-(tetrahydro-2H-pyran-4-yl)-1H-pyrazole-5-carboxylate was prepared as a white solid (1.76 g, 87%). Mass (m/z): 405.0 [M+H]⁺.

Step 4. Following General Step C, 4-((4-cyanophenyl)sulfonamido)-1-methyl-3-(tetrahydro-2H-pyran-4-yl)-1H-pyrazole-5-carboxylic acid was prepared as a yellow oil (250 mg, 78%). Mass (m/z): 391.2 [M+H]⁺.

Step 5. Following General Step D, (S)-4-((4-cyanophenyl)sulfonamido)-N-(3,3-dimethylbutan-2-yl)-1-methyl-3-(tetrahydro-2H-pyran-4-yl)-1H-pyrazole-5-carboxamide (Compound 25) was prepared as a white solid (75 mg, 24%). Mass (m/z): 474.2 [M+H]⁺.

### Compound 26: (S)-4-((4-cyclopropylphenyl)sulfonamido)-N-(3,3-dimethylbutan-2-yl)-3-(4-fluorophenyl)-1-methyl-1H-pyrazole-5-carboxamide

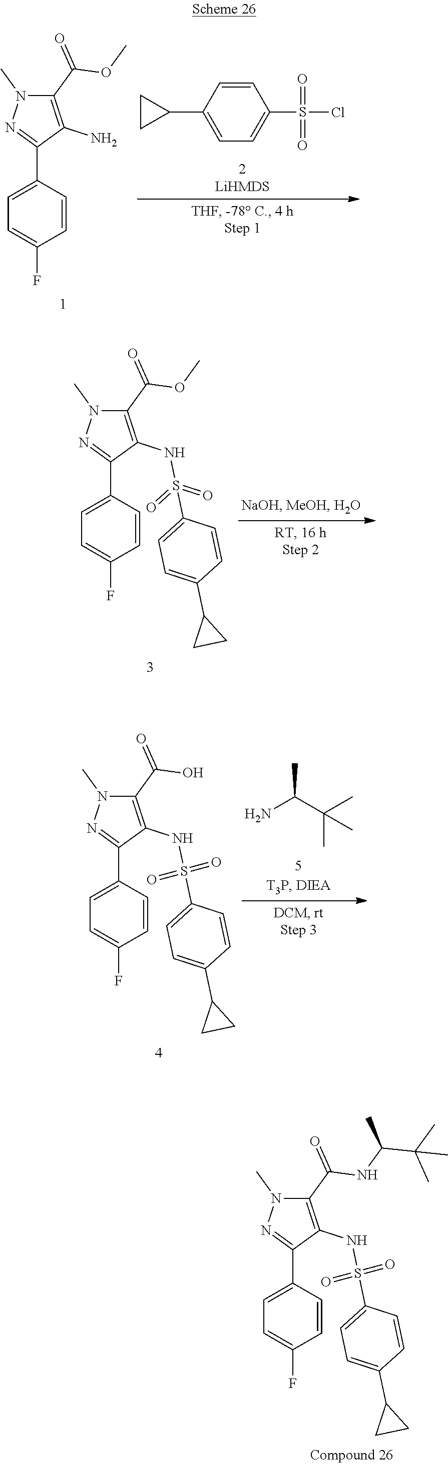

Step 1. Following General Step B1, methyl 4-((4-cyclopropylphenyl)sulfonamido)-3-(4-fluorophenyl)-1-methyl-1H-pyrazole-5-carboxylate was prepared as a white solid (370 mg, 71%). Mass (m/z): 430.0 [M+H]⁺.

Step 2. Following General Step C, 4-((4-cyclopropylphenyl)sulfonamido)-3-(4-fluorophenyl)-1-methyl-1H-pyrazole-5-carboxylic acid was prepared as a yellow solid (360 mg, 90%). Mass (m/z): 416.0 [M+H]⁺.

Step 3. Following General Step D, (S)-4-((4-cyclopropylphenyl)sulfonamido)-N-(3,3-dimethylbutan-2-yl)-3-(4-fluorophenyl)-1-methyl-1H-pyrazole-5-carboxamide (Compound 26) was prepared as a white solid (165 mg, 45%). Mass (m/z): 499.0 [M+H]⁺.

### Compound 27: (S)—N-(3,3-dimethylbutan-2-yl)-3-(4-fluorophenyl)-4-((4-methoxyphenyl)sulfonamido)-1-methyl-1H-pyrazole-5-carboxamide

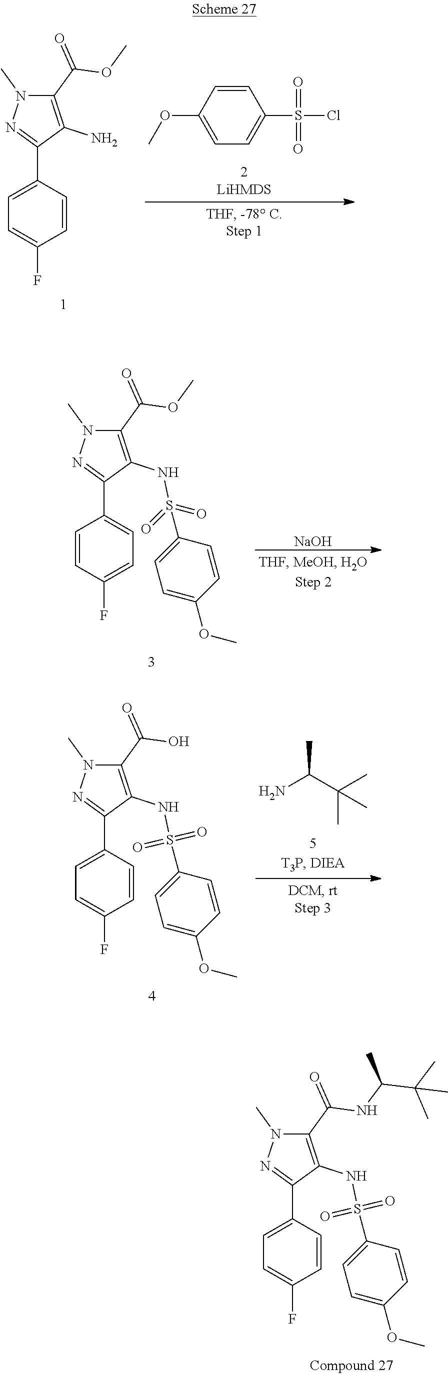

Step 1. Following General Step B1, methyl 3-(4-fluorophenyl)-4-((4-methoxyphenyl)sulfonamido)-1-methyl-1H-pyrazole-5-carboxylate was prepared as a white solid (230 mg, 43%). Mass (m/z): 420.1 [M+H]⁺.

Step 2. Following General Step C, 3-(4-fluorophenyl)-4-((4-methoxyphenyl)sulfonamido)-1-methyl-1H-pyrazole-5-carboxylic acid was prepared as a white solid (200 mg, 86%). Mass (m/z): 406.1 [M+H]⁺.

Step 3. Following General Step D, (S)—N-(3,3-dimethylbutan-2-yl)-3-(4-fluorophenyl)-4-((4-methoxyphenyl)sulfonamido)-1-methyl-1H-pyrazole-5-carboxamide (Compound 27) was prepared as a white solid (39 mg, 15%). Mass (m/z): 489.1 [M+H]⁺.

### Compound 28: (S)-4-((4-cyano-2-fluorophenyl)sulfonamido)-N-(3,3-dimethylbutan-2-yl)-3-(4-fluorophenyl)-1-methyl-1H-pyrazole-5-carboxamide

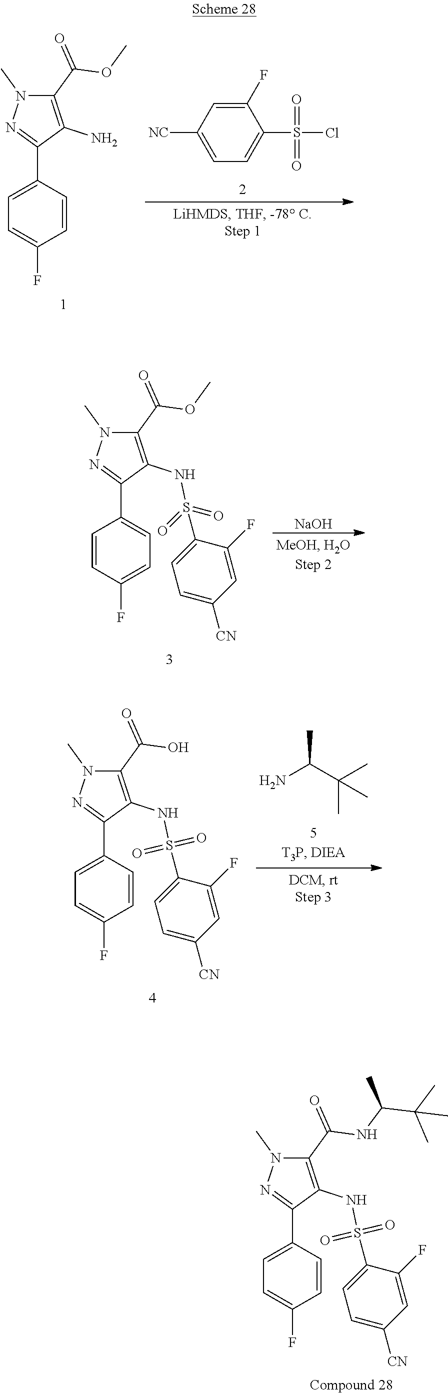

Step 1. Following General Step B1, methyl 4-((4-cyano-2-fluorophenyl)sulfonamido)-3-(4-fluorophenyl)-1-methyl-1H-pyrazole-5-carboxylate was prepared as a white solid (220 mg, 57%). Mass (m/z): 433.0 [M+H]⁺.

Step 2. Following General Step C, 4-((4-cyano-2-fluorophenyl)sulfonamido)-3-(4-fluorophenyl)-1-methyl-1H-pyrazole-5-carboxylic acid was prepared as a yellow solid (210 mg, 97%). Mass (m/z): 418.8 [M+H]⁺.

Step 3. Following General Step D, (S)-4-((4-cyano-2-fluorophenyl)sulfonamido)-N-(3,3-dimethylbutan-2-yl)-3-(4-fluorophenyl)-1-methyl-1H-pyrazole-5-carboxamide (Compound 28) was prepared as a white solid (27 mg, 11%). Mass (m/z): 501.8 [M+H]⁺.

### Compound 29: (S)-3-(4,4-difluorocyclohex-1-en-1-yl)-N-(3,3-dimethylbutan-2-yl)-1-methyl-4-((4-methylphenyl)sulfonamido)-1H-pyrazole-5-carboxamide

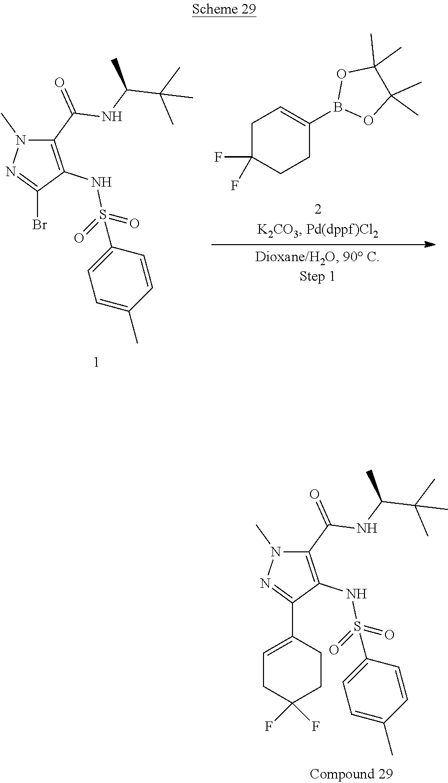 [SMILES: Cc1ccc(S(=O)(=O)Nc2c(Br)nn(C)c2C(=O)N[C@@H](C)C(C)(C)C)cc1 ; CC1(C)OB(C2=CCC(F)(F)CC2)OC1(C)C ; Cc1ccc(S(=O)(=O)Nc2c(C3=CCC(F)(F)CC3)nn(C)c2C(=O)N[C@@H](C)C(C)(C)C)cc1]

Step 1. Following General Step A, (S)-3-(4,4-difluorocyclohex-1-en-1-yl)-N-(3,3-dimethylbutan-2-yl)-1-methyl-4-((4-methylphenyl)sulfonamido)-1H-pyrazole-5-carboxamide (Compound 29) was prepared as a white solid (28 mg, 42%). Mass (m/z): 494.8 [M+H]⁺.

### Compound 30: (S)-3-(3,6-dihydro-2H-pyran-4-yl)-N-(3,3-dimethylbutan-2-yl)-1-methyl-4-((4-methylphenyl)sulfonamido)-1H-pyrazole-5-carboxamide & Compound 31: (S)—N-(3,3-dimethylbutan-2-yl)-3-(4-hydroxytetrahydro-2H-pyran-4-yl)-1-methyl-4-((4-methylphenyl)sulfonamido)-1H-pyrazole-5-carboxamide

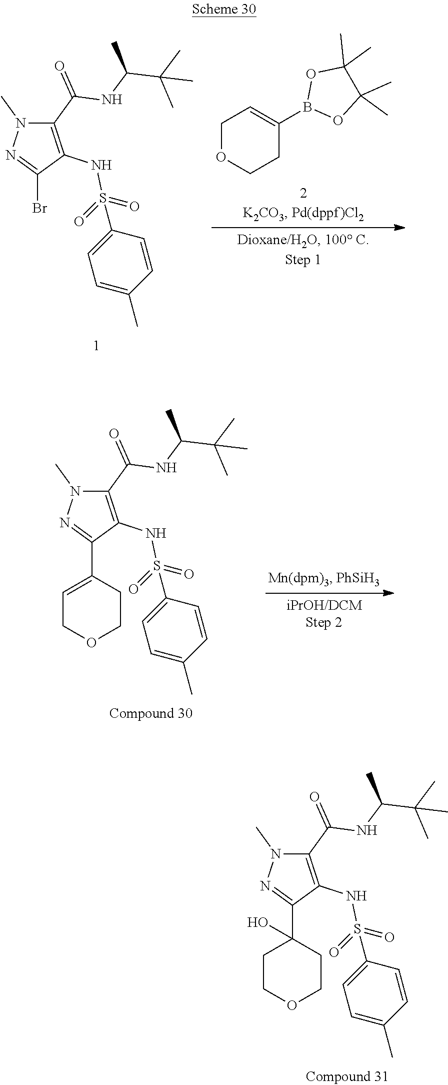 [SMILES: Cc1ccc(S(=O)(=O)Nc2c(Br)nn(C)c2C(=O)N[C@@H](C)C(C)(C)C)cc1 ; CC1(C)OB(C2=CCOCC2)OC1(C)C ; Cc1ccc(S(=O)(=O)Nc2c(C3=CCOCC3)nn(C)c2C(=O)N[C@@H](C)C(C)(C)C)cc1 ; Cc1ccc(S(=O)(=O)Nc2c(C3(O)CCOCC3)nn(C)c2C(=O)N[C@@H](C)C(C)(C)C)cc1]

Step 1. Following General Step A, (S)-3-(3,6-dihydro-2H-pyran-4-yl)-N-(3,3-dimethylbutan-2-yl)-1-methyl-4-((4-methylphenyl)sulfonamido)-1H-pyrazole-5-carboxamide (Compound 30) was prepared as a yellow solid (15 mg, 21%). Mass (m/z): 460.9 [M+H]⁺.

Step 2. Preparation of (S)—N-(3,3-dimethylbutan-2-yl)-3-(4-hydroxytetrahydro-2H-pyran-4-yl)-1-methyl-4-((4-methylphenyl)sulfonamido)-1H-pyrazole-5-carboxamide (Compound 31):

To a solution of (S)-3-(3,6-dihydro-2H-pyran-4-yl)-N-(3,3-dimethylbutan-2-yl)-1-methyl-4-((4-methylphenyl)sulfonamido)-1H-pyrazole-5-carboxamide (30.0 mg, 0.065 mmol) in the mixed solvent of i-PrOH and DCM (5 mL, 9:1 (v/v)) were added Mn(dpm)₃ (0.870 mg, 0.0014 mmol) and phenylsilane (14.1 mg, 0.130 mmol) at 0° C. The resulting solution was stirred at ambient temperature under N₂ for 16 hours, and then concentrated under vacuum. The crude residue was purified by preparative TLC (PE/EA=1:1) to give the product (S)—N-(3,3-dimethylbutan-2-yl)-3-(4-hydroxytetrahydro-2H-pyran-4-yl)-1-methyl-4-((4-methylphenyl)sulfonamido)-1H-pyrazole-5-carboxamide (Compound 31) as a white solid (12.6 mg, 36%). Mass (m/z): 460.9 [M-17]⁺.

### Compound 32: 3-(4-cyanocyclohex-1-en-1-yl)-N—((S)-3,3-dimethylbutan-2-yl)-1-methyl-4-((4-methylphenyl)sulfonamido)-1H-pyrazole-5-carboxamide

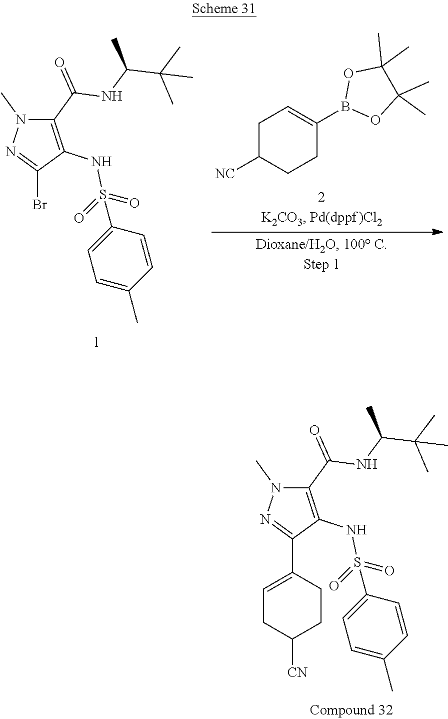 [SMILES: Cc1ccc(S(=O)(=O)Nc2c(Br)nn(C)c2C(=O)N[C@@H](C)C(C)(C)C)cc1 ; CC1(C)OB(C2=CCC(C#N)CC2)OC1(C)C ; Cc1ccc(S(=O)(=O)Nc2c(C3=CCC(C#N)CC3)nn(C)c2C(=O)N[C@@H](C)C(C)(C)C)cc1]

Step 1. Following General Step A, 3-(4-cyanocyclohex-1-en-1-yl)-N—((S)-3,3-dimethylbutan-2-yl)-1-methyl-4-((4-methylphenyl)sulfonamido)-1H-pyrazole-5-carboxamide (Compound 32) was prepared as a white solid (41 mg, 55%). Mass (m/z): 483.9 [M+H]⁺.

### Compound 33: (S)—N-(3,3-dimethylbutan-2-yl)-3-(2-(methoxymethyl)phenyl)-1-methyl-4-((4-methylphenyl)sulfonamido)-1H-pyrazole-5-carboxamide

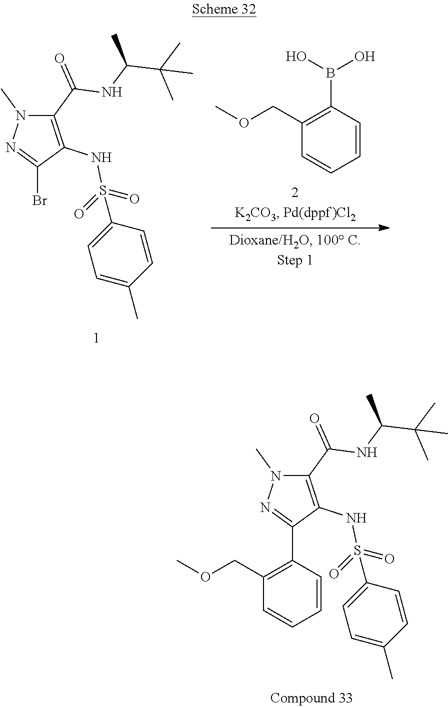

Step 1. Following General Step A, (S)—N-(3,3-dimethylbutan-2-yl)-3-(2-(methoxymethyl)phenyl)-1-methyl-4-((4-methylphenyl)sulfonamido)-1H-pyrazole-5-carboxamide (Compound 33) was prepared as a white solid (35 mg, 38%). Mass (m/z): 499.0 [M+H]⁺.

### Compound 34: (S)—N-(3,3-dimethylbutan-2-yl)-1-methyl-4-((4-methylphenyl)sulfonamido)-3-(3-(trifluoromethoxy)phenyl)-1H-pyrazole-5-carboxamide

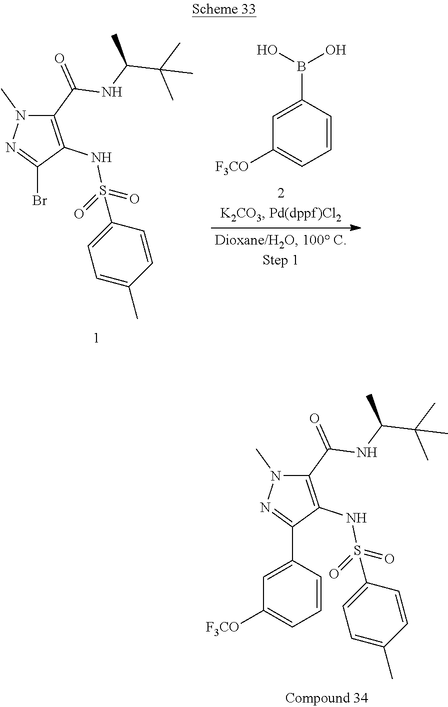 [SMILES: Cc1ccc(S(=O)(=O)Nc2c(Br)nn(C)c2C(=O)N[C@@H](C)C(C)(C)C)cc1 ; OB(O)c1cccc(OC(F)(F)F)c1 ; Cc1ccc(S(=O)(=O)Nc2c(-c3cccc(OC(F)(F)F)c3)nn(C)c2C(=O)N[C@@H](C)C(C)(C)C)cc1]

Step 1. Following General Step A, (S)—N-(3,3-dimethylbutan-2-yl)-1-methyl-4-((4-methylphenyl)sulfonamido)-3-(3-(trifluoromethoxy)phenyl)-1H-pyrazole-5-carboxamide (Compound 34) was prepared as a white solid (25 mg, 25%). Mass (m/z): 538.9 [M+H]⁺.

### Compound 35: (S)—N-(3,3-dimethylbutan-2-yl)-3-(3-isobutoxyphenyl)-1-methyl-4-((4-methylphenyl)sulfonamido)-1H-pyrazole-5-carboxamide

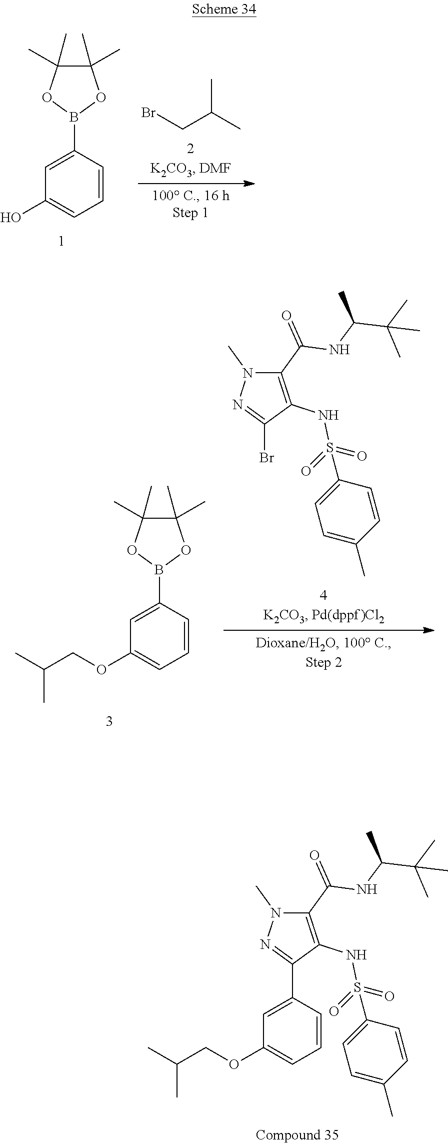

Step 1. Preparation of 4,4,5,5-tetramethyl-2-[3-(2-methylpropoxy)phenyl]-1,3,2-dioxaborolane: To a mixture of 3-(4,4,5,5-tetramethyl-1,3,2-dioxaborolan-2-yl)phenol (1.00 g, 4.5 mmol) in DMF (10 mL) were added K₂CO₃ (1.87 g, 13.5 mmol) and 1-bromo-2-methylpropane (0.740 g, 5.4 mmol). The resulting mixture was stirred at 100° C. for 16 hours, and then diluted with H₂O (50 mL). The aqueous solution was extracted with EA (50 mL×3). The combined organic layers were washed with brine (50 mL×3), dried over Na₂SO₄, and then filtered. The filtrate was concentrated under reduced pressure. The crude residue was purified by flash column chromatography (PE:EA=0-7%) to give the product 4,4,5,5-tetramethyl-2-[3-(2-methylpropoxy)phenyl]-1,3,2-dioxaborolane as a colorless oil (600 mg, 44%). Mass (m/z): 277.0 [M+H]⁺.

Step 2. Following General Step A, (S)—N-(3,3-dimethylbutan-2-yl)-3-(3-isobutoxyphenyl)-1-methyl-4-((4-methylphenyl)sulfonamido)-1H-pyrazole-5-carboxamide (Compound 35) was prepared as a white solid (38 mg, 29%). Mass (m/z): 526.9 [M+H]⁺.

### Compound 36: (S)-4-((4-chlorophenyl)sulfonamido)-3-(4,4-difluorocyclohex-1-en-1-yl)-N-(3,3-dimethylbutan-2-yl)-1-methyl-1H-pyrazole-5-carboxamide & Compound 37: (S)-4-((4-chlorophenyl)sulfonamido)-3-(4,4-difluorocyclohexyl)-N-(3,3-dimethylbutan-2-yl)-1-methyl-1H-pyrazole-5-carboxamide

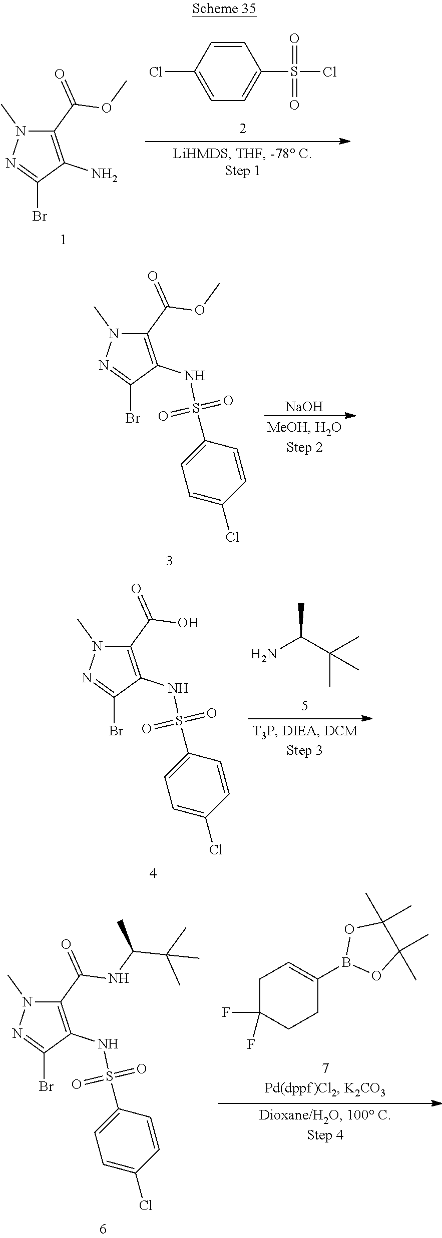

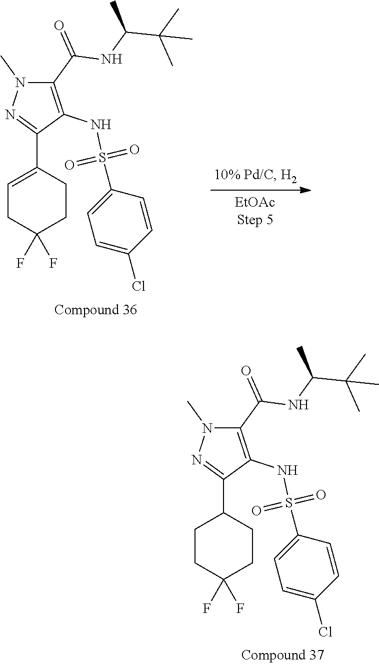 [SMILES: C[C@H](NC(=O)c1c(NS(=O)(=O)c2ccc(Cl)cc2)c(C2=CCC(F)(F)CC2)nn1C)C(C)(C)C ; C[C@H](NC(=O)c1c(NS(=O)(=O)c2ccc(Cl)cc2)c(C2CCC(F)(F)CC2)nn1C)C(C)(C)C]

Step 1. Following General Step B1, methyl 3-bromo-4-((4-chlorophenyl)sulfonamido)-1-methyl-1H-pyrazole-5-carboxylate was prepared as a yellow solid (200 mg, 19%). Mass (m/z): 407.6 [M+H]⁺.

Step 2. Following General Step C, 3-bromo-4-((4-chlorophenyl)sulfonamido)-1-methyl-1H-pyrazole-5-carboxylic acid was prepared as a white solid (150 mg, 78%). Mass (m/z): 393.7 [M+H]⁺.

Step 3. Following General Step D, (S)-3-bromo-4-((4-chlorophenyl)sulfonamido)-N-(3,3-dimethylbutan-2-yl)-1-methyl-1H-pyrazole-5-carboxamide was prepared as a white solid (150 mg, 74%). Mass (m/z): 476.6 [M+H]⁺.

Step 4. Following General Step A, (S)-4-((4-chlorophenyl)sulfonamido)-3-(4,4-difluorocyclohex-1-en-1-yl)-N-(3,3-dimethylbutan-2-yl)-1-methyl-1H-pyrazole-5-carboxamide (Compound 36) was prepared as a white solid (30 mg, 25%). Mass (m/z): 514.8 [M+H]⁺.

Step 5. Following General Step I, (S)-4-((4-chlorophenyl)sulfonamido)-3-(4,4-difluorocyclohexyl)-N-(3,3-dimethylbutan-2-yl)-1-methyl-1H-pyrazole-5-carboxamide (Compound 37) was prepared as a white solid (7 mg, 27%). Mass (m/z): 516.9 [M+H]⁺.

### Compound 38: (S)-4-((4-chlorophenyl)sulfonamido)-N-(3,3-dimethylbutan-2-yl)-3-(4-fluorophenyl)-1-methyl-1H-pyrazole-5-carboxamide

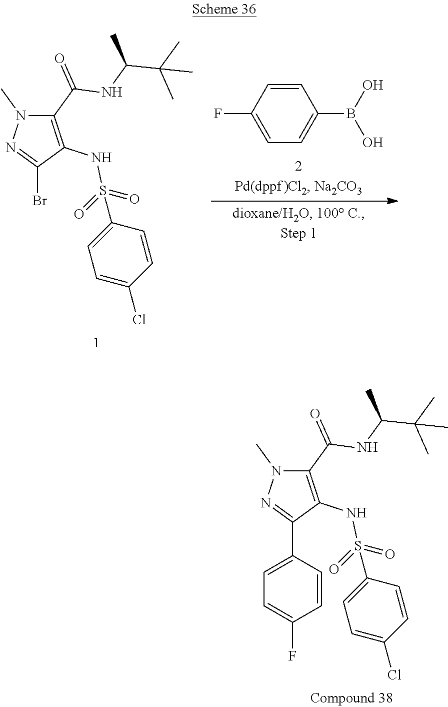 [SMILES: C[C@H](NC(=O)c1c(NS(=O)(=O)c2ccc(Cl)cc2)c(Br)nn1C)C(C)(C)C ; OB(O)c1ccc(F)cc1 ; C[C@H](NC(=O)c1c(NS(=O)(=O)c2ccc(Cl)cc2)c(-c2ccc(F)cc2)nn1C)C(C)(C)C]

Step 1. Following General Step A, (S)-4-((4-chlorophenyl)sulfonamido)-N-(3,3-dimethylbutan-2-yl)-3-(4-fluorophenyl)-1-methyl-1H-pyrazole-5-carboxamide (Compound 38) was prepared as a white solid (20 mg, 19%). Mass (m/z): 493.1 [M+H]⁺.

### Compound 39: (S)-4-((4-chlorophenyl)sulfonamido)-3-(2-(difluoromethyl)phenyl)-N-(3,3-dimethylbutan-2-yl)-1-methyl-1H-pyrazole-5-carboxamide

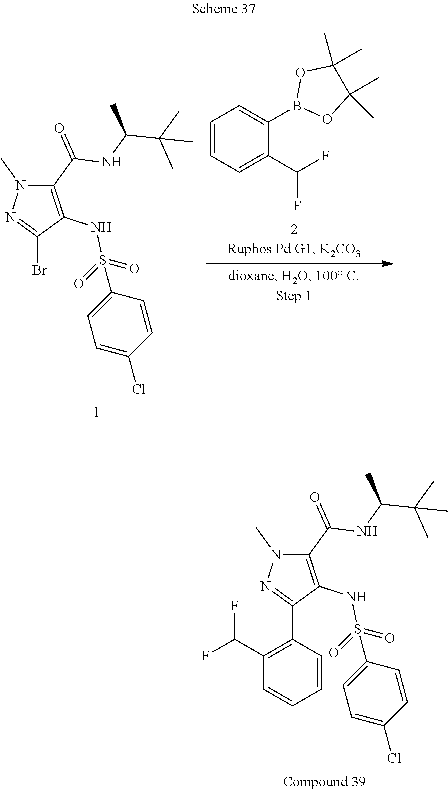

Step 1. Following General Step A, (S)-4-((4-chlorophenyl)sulfonamido)-3-(2-(difluoromethyl)phenyl)-N-(3,3-dimethylbutan-2-yl)-1-methyl-1H-pyrazole-5-carboxamide (Compound 39) was prepared as a yellow solid (5.1 mg, 4%). Mass (m/z): 525.1 [M+H]⁺.

### Compound 40: (S)-4-((4-chlorophenyl)sulfonamido)-N-(3,3-dimethylbutan-2-yl)-3-(5-(methoxymethyl)pyridin-3-yl)-1-methyl-1H-pyrazole-5-carboxamide

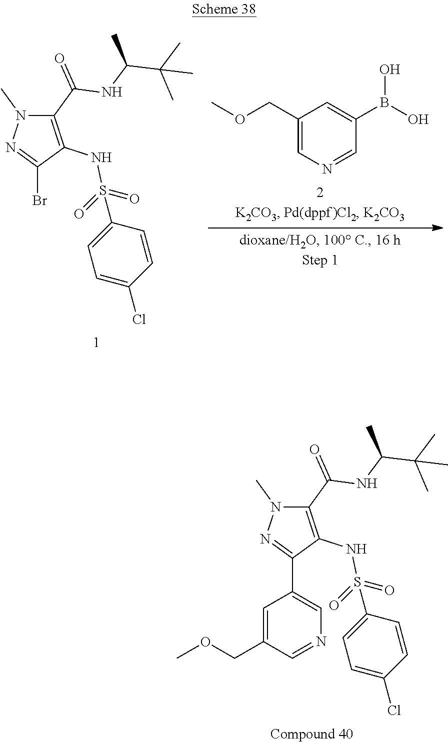

Step 1. Following General Step A, (S)-4-((4-chlorophenyl)sulfonamido)-N-(3,3-dimethylbutan-2-yl)-3-(5-(methoxymethyl)pyridin-3-yl)-1-methyl-1H-pyrazole-5-carboxamide (Compound 40) was prepared as a white solid (34.0 mg, 57%). Mass (m/z): 519.8 [M+H]⁺.

### Compound 41: (S)-4-((4-cyanophenyl)sulfonamido)-3-(2,4-difluorophenyl)-N-(3,3-dimethylbutan-2-yl)-1-methyl-1H-pyrazole-5-carboxamide

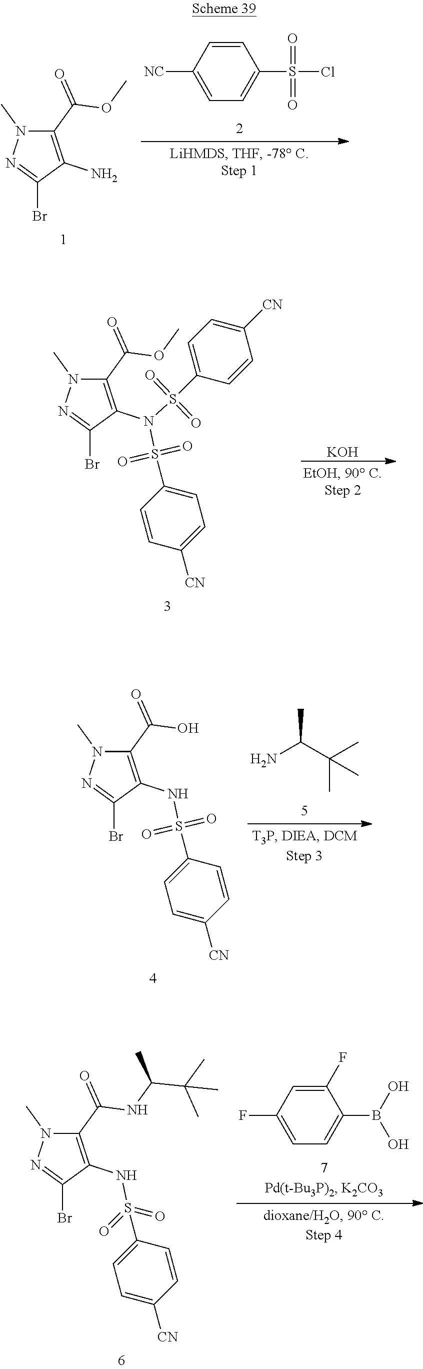

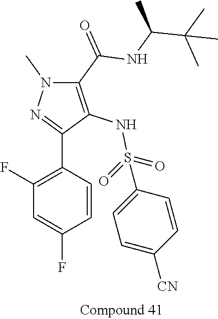 [SMILES: C[C@H](NC(=O)c1c(NS(=O)(=O)c2ccc(C#N)cc2)c(-c2ccc(F)cc2F)nn1C)C(C)(C)C]

Step 1. Following General Step B1, methyl 3-bromo-4-((4-cyano-N-((4-cyanophenyl)sulfonyl)phenyl)sulfonamido)-1-methyl-1H-pyrazole-5-carboxylate was prepared as a yellow solid (11 g, 82%). Mass (m/z): 585.6 [M+Na]⁺.

Step 2. Following General Step C, 3-bromo-4-((4-cyanophenyl)sulfonamido)-1-methyl-1H-pyrazole-5-carboxylic acid was prepared as a yellow solid (8.2 g, 96%). Mass (m/z): 384.8 [M+H]⁺.

Step 3. Following General Step D, (S)-3-bromo-4-((4-cyanophenyl)sulfonamido)-N-(3,3-dimethylbutan-2-yl)-1-methyl-1H-pyrazole-5-carboxamide was prepared as a yellow solid (5.3 g, 46%). Mass (m/z): 467.9 [M+H]⁺.

Step 4. Following General Step A, (S)-4-((4-cyanophenyl)sulfonamido)-3-(2,4-difluorophenyl)-N-(3,3-dimethylbutan-2-yl)-1-methyl-1H-pyrazole-5-carboxamide (Compound 41) was prepared as a white solid (360 mg, 23%). Mass (m/z): 502.1 [M+H]⁺.

### Compound 42: (S)-4-((4-cyanophenyl)sulfonamido)-N-(3,3-dimethylbutan-2-yl)-3-(2-(methoxymethyl)pyridin-3-yl)-1-methyl-1H-pyrazole-5-carboxamide

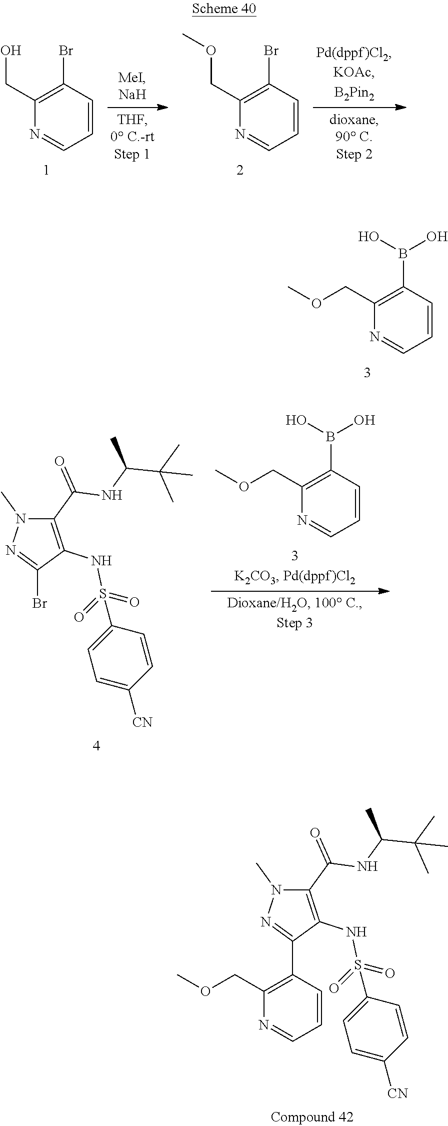

### Step 1. Preparation of 3-bromo-2-(methoxymethyl)pyridine

To a solution of (3-bromopyridin-2-yl)methanol (500 mg, 2.7 mmol) in THF (10 mL) was added NaH (60% in mineral oil, 128 mg, 3.2 mmol) at 0° C. under N₂. The resulting mixture was stirred at 0° C. for 30 minutes before the addition of Mel (417 mg, 2.9 mmol). The solution was further stirred at ambient temperature under N₂ for 16 hrs, and then diluted with H₂O (20 mL). The aqueous solution was extracted with EA (20 mL×3). The combined organic layers were washed with brine (30 mL×2), dried over with anhydrous Na₂SO₄, and then filtered. The filtrate was concentration under vacuum to give the product 3-bromo-2-(methoxymethyl)pyridine as a colorless oil (504 mg, 93%). Mass (m/z): 202.0 [M+H]⁺.

### Step 2. Preparation of (2-(methoxymethyl)pyridin-3-yl)boronic acid

To a solution of 3-bromo-2-(methoxymethyl)pyridine (500 mg, 2.47 mmol) in dioxane (5 mL) were added B₂Pin₂ (943 mg, 3.71 mmol), KOAc (1.21 g, 12.4 mmol) and Pd(dppf)Cl₂ (181 mg, 0.24 mmol). The resulting mixture was stirred at 90° C. under N₂ for 1 hour, and then concentrated under vacuum. The crude residue was purified by flash column chromatography (PE/EA=0-50%) to give the product (2-(methoxymethyl)pyridin-3-yl)boronic acid as a yellow oil (210 mg, 50%). Mass (m z): 168.1 [M+H]⁺.

Step 3. Following General Step A, (S)-4-((4-cyanophenyl)sulfonamido)-N-(3,3-dimethylbutan-2-yl)-3-(2-(methoxymethyl)pyridin-3-yl)-1-methyl-1H-pyrazole-5-carboxamide (Compound 42) was prepared as a white solid (12 mg, 13%). Mass (m/z): 510.8 [M+H]⁺.

### Compound 43: (S)-4-((4-cyanophenyl)sulfonamido)-3-(4,4-difluorocyclohex-1-en-1-yl)-N-(3,3-dimethylbutan-2-yl)-1-methyl-1H-pyrazole-5-carboxamide & Compound 44: (S)-4-((4-cyanophenyl)sulfonamido)-3-(4,4-difluorocyclohexyl)-N-(3,3-dimethylbutan-2-yl)-1-methyl-1H-pyrazole-5-carboxamide

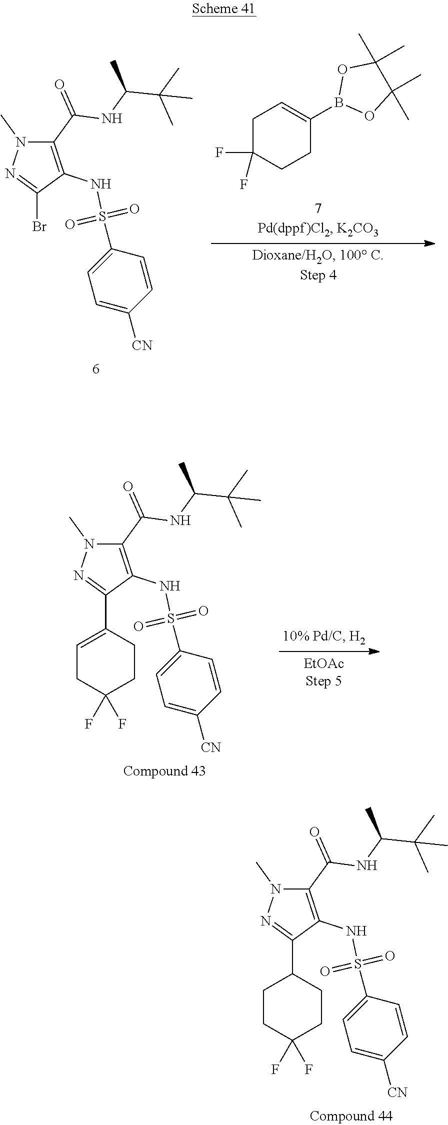 [SMILES: C[C@H](NC(=O)c1c(NS(=O)(=O)c2ccc(C#N)cc2)c(Br)nn1C)C(C)(C)C ; CC1(C)OB(C2=CCC(F)(F)CC2)OC1(C)C ; C[C@H](NC(=O)c1c(NS(=O)(=O)c2ccc(C#N)cc2)c(C2=CCC(F)(F)CC2)nn1C)C(C)(C)C ; C[C@H](NC(=O)c1c(NS(=O)(=O)c2ccc(C#N)cc2)c(C2CCC(F)(F)CC2)nn1C)C(C)(C)C]

Step 1. Following General Step A, (S)-4-((4-cyanophenyl)sulfonamido)-3-(4,4-difluorocyclohex-1-en-1-yl)-N-(3,3-dimethylbutan-2-yl)-1-methyl-1H-pyrazole-5-carboxamide (Compound 43) was prepared as a white solid (28 mg, 13%). Mass (m/z): 505.8 [M+H]⁺.

Step 2. Following General Step I, (S)-4-((4-cyanophenyl)sulfonamido)-3-(4,4-difluorocyclohexyl)-N-(3,3-dimethylbutan-2-yl)-1-methyl-1H-pyrazole-5-carboxamide (Compound 44) was prepared as a white solid (8.5 mg, 11%). Mass (m/z): 507.9 [M+H]⁺.

### Example 2. In Vitro Assay for Detecting and Measuring Modulation of hFPR1 by Compounds

Cyclic Adenosine Monophosphate (cAMP) Assay

CHO-K1 cells stably overexpressing hFPR1 were plated into 384-well cell culture plates (2,000/well/5 μL) in 1× Stimulation Buffer (LANCE Ultra cAMP Kit, Perkin Elmer, TRF0263). 1 μL compound solution (prepared in ddH₂O containing 0.2% DMSO) was added to each well. Plates were centrifuged briefly, and cells were incubated at 37° C. in 5% CO₂ for 10 minutes. 4 μL solution containing 2.5 μM Forskolin and 0.25 nM WKYMVm (an FPR1 agonist, GLPBIO, GC15140) was then added to each well. 5 μL of Eu-cAMP solution (diluted to manufacturer's recommended working concentration in Detection Buffer, LANCE Ultra cAMP Kit) and 5 μL of ULight-anti-cAMP solution (diluted in Detection Buffer, LANCE Ultra cAMP Kit) were added to each well. Plates were centrifuged briefly, and cells were incubated at room temperature for 1 hour. Fluorescence emission at 665 nm and 620 nm from the samples was measured by a Perkin Elmer Envision instrument.

Reactive Oxygen Species (ROS) Assay

Human whole blood was collected, and ACK lysis buffer (ThermoFisher, A1049201) was added at 3 times the volume of the blood sample to lyse the red blood cells. The sample was incubated on ice for 15 minutes, and gently mixed twice during the incubation. 10 mL DPBS was then added, and the sample was centrifuged for 8 minutes. After removing the supernatant, remaining cells were resuspended in RPMI1640 medium containing 3% FBS and centrifuged again for 10 minutes. After removing the supernatant, cells were again resuspended in RPMI1640 medium containing 3% FBS. Neutrophil cell count was determined using FACS analysis by APC-labeled CD11b antibody (Biolegend, 101211) and FITC-labeled CD66b antibody (Biolegend, 305104). Neutrophils were then plated into 96-well plates (300,000/well). Plates were centrifuged and supernatant was removed. Cells were resuspended in 80 μL of compound solution (prepared in RPMI1640 medium containing 3% FBS), then incubated at room temperature for 15 minutes. 20 μL solution containing DCFH-DA (at the manufacturer's recommended concentration, Yeasen Biotechnology, 50101ES01) and fMLP (500 nM) was then added. Cells were incubated at 37° C. in 5% CO₂ for 20 minutes away from light. After incubation, cells were placed on ice for 5 minutes, washed twice with 200 μL ice-cold DPBS, then resuspended in ice-cold DPBS at 100 L/well. Fluorescence from each sample was measured at 485 nm excitation and 535 nm emission.

The effects of the compounds of the present disclosure on regulating the FPR1-mediated cell signaling were measured by monitoring the cellular cAMP level change and anti-reactive oxygen species formation in human neutrophils in the presence of an FPR1 agonist, respectively, as illustrated by the examples in Table 1.

***: IC₅₀<100 nM; **: 100 nM<IC₅₀<1 μM; *; 1 μM<IC₅₀<25 μM

### NA: Not Active up to 25 μM; ND: Not Determined

TABLE 1
cAMP and anti-ROS IC₅₀ values for the tested compounds

| Compound Number | cAMP IC₅₀ (nM) | Anti-ROS IC₅₀ (nM) |
|---|---|---|
| 1 | ** | ** |
| 3 | NA | ND |
| 5 | NA | ND |
| 6 | ** | *** |
| 9 | ** | ** |
| 10 | NA | ND |
| 11 | NA | ND |
| 15 | NA | ** |
| 16 | * | ** |
| 18 | NA | ND |
| 19 | ** | *** |
| 20 | ** | *** |
| 21 | *** | *** |
| 22 | ** | *** |
| 23 | * | * |
| 24 | * | * |
| 25 | ** | ** |
| 26 | * | ** |
| 27 | ** | ** |
| 28 | ** | ** |
| 29 | NA | ND |
| 30 | * | ** |
| 31 | NA | ND |
| 32 | * | ND |
| 33 | ** | ** |
| 34 | NA | ND |
| 35 | NA | ND |
| 36 | NA | ND |
| 37 | ** | ** |
| 38 | ** | ** |
| 39 | ** | ** |
| 40 | * | ND |
| 41 | ** | ** |
| 42 | NA | NA |
| 43 | * | ** |
| 44 | ** | ** |

### Other Embodiments

The present disclosure provides merely exemplary embodiments. One skilled in the art will readily recognize from the present disclosure and claims, that various changes, modifications and variations can be made therein without departing from the spirit and scope of the present disclosure as defined in the following claims.

1. A compound of Formula (I): 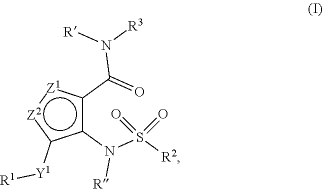
a tautomer thereof, a deuterated derivative of the compound or the tautomer, or a pharmaceutically acceptable salt the foregoing, wherein:
(i) each Z¹ and Z² is independently chosen from O, S, N, NR⁴, C(R⁴)₂, and CR⁴, and at least one of Z¹ and Z² is O, S, N, or NR⁴;
wherein R⁴ is chosen from hydrogen, linear, branched, and cyclic alkyl groups, carbocyclic groups, heterocyclic groups, aryl groups, and heteroaryl groups;
(ii) Y¹ is absent or is chosen from O, S, and NR⁵;
wherein R⁵ is chosen from hydrogen, linear, branched, and cyclic alkyl groups, carbocyclic groups, heterocyclic groups, aryl groups, and heteroaryl groups;
(iii) R¹ is chosen from linear, branched, and cyclic alkyl groups, alkenyl groups, carbocyclic groups, heterocyclic groups, linear, branched, and cyclic alkenyl groups, linear and branched heteroalkenyl groups, aryl groups, and heteroaryl groups; or R¹ and Z², together with the atoms to which they are attached, form a cycloalkyl group, carbocyclic group, a heterocyclic group, an aryl group, or a heteroaryl group;
(iv) each R² and R³ is independently chosen from linear, branched, and cyclic alkyl groups, carbocyclic groups, heterocyclic groups, aryl groups, and heteroaryl groups;
(v) each R′ and R″ is independently chosen from hydrogen, linear, branched, and cyclic alkyl groups, carbocyclic groups, heterocyclic groups, aryl groups, and heteroaryl groups;
wherein the linear, branched, and cyclic alkyl groups, linear, branched, and cyclic alkenyl groups, carbocyclic groups, linear and branched heteroalkenyl groups, heterocyclic groups, aryl groups, and heteroaryl groups are optionally substituted with at least one group chosen from the following groups:
halogen groups,
hydroxy,
thiol,
amino,
cyano,
—OC(O)C₁-C₆ linear, branched, and cyclic alkyl groups,
—C(O)OC₁-C₆ linear, branched, and cyclic alkyl groups,
—NHC₁-C₆ linear, branched, and cyclic alkyl groups,
—N(C₁-C₆ linear, branched, and cyclic alkyl groups)₂,
—NHC(O)C₁-C₆ linear, branched, and cyclic alkyl groups,
—C(O)NHC₁-C₆ linear, branched, and cyclic alkyl groups,
—NHaryl groups,
—N(aryl groups)₂,
—NHC(O)aryl groups,
—C(O)NHaryl groups,
—NHheteroaryl groups,
—N(heteroaryl groups)₂,
—NHC(O)heteroaryl groups,
—C(O)NHheteroaryl groups,
C₁-C₆ linear, branched, and cyclic alkyl groups,
C₂-C₆ linear, branched, and cyclic alkenyl groups,
C₁-C₆ linear, branched, and cyclic hydroxyalkyl groups,
C₁-C₆ linear, branched, and cyclic alkoxy groups,
C₁-C₆ linear, branched, and cyclic thioalkyl groups,
C₁-C₆ linear, branched, and cyclic haloalkyl groups,
C₁-C₆ linear, branched, and cyclic haloaminoalkyl groups,
C₁-C₆ linear, branched, and cyclic halothioalkyl groups,
C₁-C₆ linear, branched, and cyclic haloalkoxy groups,
benzyloxy, benzylamino, and benzylthio groups,
3 to 6-membered heterocycloalkenyl groups,
3 to 6-membered heterocyclic groups, and
5 and 6-membered heteroaryl groups.

2. The compound, tautomer, deuterated derivative, or pharmaceutically acceptable salt of claim 1, wherein R¹ is chosen from cyclic alkyl groups, carbocyclic groups, heterocyclic groups, alkenyl groups, aryl groups, and heteroaryl groups; R² is an aryl group; and R³ is chosen from linear and branched alkyl groups.

3. The compound, tautomer, deuterated derivative, or pharmaceutically acceptable salt of claim 1 or 2, wherein R¹ is chosen from optionally substituted 5 and 6-membered aryl groups.

4. The compound, tautomer, deuterated derivative, or pharmaceutically acceptable salt of claim 3, wherein R¹ is chosen from 5 and 6-membered aryl groups substituted with at least one halogen group.

5. The compound, tautomer, deuterated derivative, or pharmaceutically acceptable salt of claim 4, wherein R¹ is chosen from 5 and 6-membered aryl groups substituted with at least one fluoro.

6. The compound, tautomer, deuterated derivative, or pharmaceutically acceptable salt of claim 4, wherein R¹ is chosen from 5 and 6-membered aryl groups substituted with at least one chloro.

7. The compound, tautomer, deuterated derivative, or pharmaceutically acceptable salt of claim 1 or 2, wherein R¹ is chosen from optionally substituted 5 and 6-membered heteroaryl groups.

8. The compound, tautomer, deuterated derivative, or pharmaceutically acceptable salt of claim 7, wherein R¹ is chosen from 5 and 6-membered heteroaryl groups substituted with at least one halogen group.

9. The compound, tautomer, deuterated derivative, or pharmaceutically acceptable salt of claim 8, wherein R¹ is chosen from 5 and 6-membered heteroaryl groups substituted with at least one fluoro.

10. The compound, tautomer, deuterated derivative, or pharmaceutically acceptable salt of claim 8, wherein R¹ is chosen from 5 and 6-membered heteroaryl groups substituted with at least one chloro.

11. The compound, tautomer, deuterated derivative, or pharmaceutically acceptable salt of claim 1 or 2, wherein R¹ is chosen from optionally substituted 3 to 6-membered cyclic alkyl groups.

12. The compound, tautomer, deuterated derivative, or pharmaceutically acceptable salt of claim 11, wherein R¹ is chosen from 3 to 6-membered cyclic alkyl groups substituted with at least one alkoxy group.

13. The compound, tautomer, deuterated derivative, or pharmaceutically acceptable salt of claim 12, wherein R¹ is chosen from 3 to 6-membered cyclic alkyl groups substituted with at least one methoxy group.

14. The compound, tautomer, deuterated derivative, or pharmaceutically acceptable salt of claim 1 or 2, wherein R¹ is chosen from optionally substituted 3 to 6-membered carbocyclic, optionally substituted alkenyl groups, and optionally substituted heterocyclic groups.

15. The compound, tautomer, deuterated derivative, or pharmaceutically acceptable salt of any one of claims 1 to 14, wherein R² is chosen from optionally substituted 5 and 6-membered heteroaryl groups.

16. The compound, tautomer, deuterated derivative, or pharmaceutically acceptable salt of any one of claims 1 to 15, wherein R² is chosen from 5 and 6-membered heteroaryl groups substituted with at least one halogen group.

17. The compound, tautomer, deuterated derivative, or pharmaceutically acceptable salt of claim 16, wherein R² is chosen from 5 and 6-membered heteroaryl groups substituted with at least one fluoro.

18. The compound, tautomer, deuterated derivative, or pharmaceutically acceptable salt of claim 16, wherein R² is chosen from 5 and 6-membered heteroaryl groups substituted with at least one chloro.

19. The compound, tautomer, deuterated derivative, or pharmaceutically acceptable salt of any one of claims 1 to 15, wherein R² is chosen from 5 and 6-membered heteroaryl groups substituted with at least one cyano.

20. The compound, tautomer, deuterated derivative, or pharmaceutically acceptable salt of any one of claims 1 to 19, wherein R² is chosen from 5 and 6-membered heteroaryl groups substituted with at least one group chosen from C₁-C₆ linear, C₃-C₆ branched, and C₃-C₆ cyclic alkyl groups.

21. The compound, tautomer, deuterated derivative, or pharmaceutically acceptable salt of any one of claims 1 to 20, wherein R³ is chosen from optionally substituted C₂-C₁₀ linear and C₂-C₁₀ branched alkyl groups.

22. The compound, tautomer, deuterated derivative, or pharmaceutically acceptable salt of claim 21, wherein R³ is chosen from methyl, ethyl, propyl, isopropyl, butyl, iso-butyl, sec-butyl, pentyl, iso-pentyl, sec-pentyl, neo-pentyl, and 1,2,2-trimethylpropyl.

23. A compound of Formula (IIA): 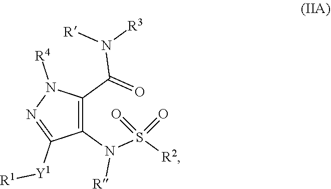
a tautomer thereof, a deuterated derivative of the compound or the tautomer, or a pharmaceutically acceptable salt of the foregoing, wherein:
(i) Y¹ is absent or is chosen from a bond, O, S, and NR⁵;
wherein R⁵ is chosen from hydrogen, linear, branched, and cyclic alkyl groups, carbocyclic groups, heterocyclic groups, aryl groups, and heteroaryl groups;
(ii) R¹ is chosen from linear, branched, and cyclic alkyl groups, alkenyl groups, carbocyclic groups, heterocyclic groups, linear, branched, and cyclic alkenyl groups, linear and branched heteroalkenyl groups, aryl groups, and heteroaryl groups;
(iii) each R² and R³ is independently chosen from linear, branched, and cyclic alkyl groups, carbocyclic groups, heterocyclic groups, aryl groups, and heteroaryl groups;
(iv) R⁴ is chosen from hydrogen, linear, branched, and cyclic alkyl groups, carbocyclic groups, heterocyclic groups, aryl groups, and heteroaryl groups;
(v) each R′ and R″ is independently chosen from hydrogen, linear, branched, and cyclic alkyl groups, carbocyclic groups, heterocyclic groups, aryl groups, and heteroaryl groups;
wherein the linear, branched, and cyclic alkyl groups, linear, branched, and cyclic alkenyl groups, linear and branched heteroalkenyl groups, carbocyclic groups, heterocyclic groups, aryl groups, and heteroaryl groups are optionally substituted with at least one group chosen from the following groups:
halogen groups,
hydroxy,
thiol,
amino,
cyano,
—OC(O)C₁-C₆ linear, branched, and cyclic alkyl groups,
—C(O)OC₁-C₆ linear, branched, and cyclic alkyl groups,
—NHC₁-C₆ linear, branched, and cyclic alkyl groups,
—N(C₁-C₆ linear, branched, and cyclic alkyl groups)₂,
—NHC(O)C₁-C₆ linear, branched, and cyclic alkyl groups,
—C(O)NHC₁-C₆ linear, branched, and cyclic alkyl groups,
—NHaryl groups,
—N(aryl groups)₂,
—NHC(O)aryl groups,
—C(O)NHaryl groups,
—NHheteroaryl groups,
—N(heteroaryl groups)₂,
—NHC(O)heteroaryl groups,
—C(O)NHheteroaryl groups,
C₁-C₆ linear, branched, and cyclic alkyl groups,
C₂-C₆ linear, branched, and cyclic alkenyl groups,
C₁-C₆ linear, branched, and cyclic hydroxyalkyl groups,
C₁-C₆ linear, branched, and cyclic alkoxy groups,
C₁-C₆ linear, branched, and cyclic thioalkyl groups,
C₁-C₆ linear, branched, and cyclic haloalkyl groups,
C₁-C₆ linear, branched, and cyclic haloaminoalkyl groups,
C₁-C₆ linear, branched, and cyclic halothioalkyl groups,
C₁-C₆ linear, branched, and cyclic haloalkoxy groups,
benzyloxy, benzylamino, and benzylthio groups,
3 to 6-membered heterocycloalkenyl groups,
3 to 6-membered heterocyclic groups, and
5 and 6-membered heteroaryl groups.

24. A compound of Formula (IIB): 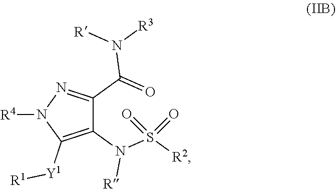
a tautomer thereof, a deuterated derivative of the compound or the tautomer, or a pharmaceutically acceptable salt of the foregoing, wherein:
(i) Y¹ is absent or is chosen from a bond, O, S, and NR⁵;
wherein R⁵ is chosen from hydrogen, linear, branched, and cyclic alkyl groups, carbocyclic groups, heterocyclic groups, aryl groups, and heteroaryl groups;
(ii) R¹ is chosen from linear, branched, and cyclic alkyl groups, alkenyl groups, carbocyclic groups, heterocyclic groups, linear, branched, and cyclic alkenyl groups, linear and branched heteroalkenyl groups, aryl groups, and heteroaryl groups; or R¹ and NR⁴, together with the atoms to which they are attached, form a cycloalkyl group, a carbocyclic group, a heterocyclic group, an aryl group, or a heteroaryl group;
(iii) each R² and R³ is independently chosen from linear, branched, and cyclic alkyl groups, carbocyclic groups, heterocyclic groups, aryl groups, and heteroaryl groups;
(iv) R⁴ is chosen from hydrogen, linear, branched, and cyclic alkyl groups, heterocyclic groups, aryl groups, and heteroaryl groups;
(v) each R′ and R″ is independently chosen from hydrogen, linear, branched, and cyclic alkyl groups, carbocyclic groups, heterocyclic groups, aryl groups, and heteroaryl groups;
wherein the linear, branched, and cyclic alkyl groups, linear, branched, and cyclic alkenyl groups, linear and branched heteroalkenyl groups, carbocyclic groups, heterocyclic groups, aryl groups, and heteroaryl groups are optionally substituted with at least one group chosen from the following groups:
halogen groups,
hydroxy,
thiol,
amino,
cyano,
—OC(O)C₁-C₆ linear, branched, and cyclic alkyl groups,
—C(O)OC₁-C₆ linear, branched, and cyclic alkyl groups,
—NHC₁-C₆ linear, branched, and cyclic alkyl groups,
—N(C₁-C₆ linear, branched, and cyclic alkyl groups)₂,
—NHC(O)C₁-C₆ linear, branched, and cyclic alkyl groups,
—C(O)NHC₁-C₆ linear, branched, and cyclic alkyl groups,
—NHaryl groups,
—N(aryl groups)₂,
—NHC(O)aryl groups,
—C(O)NHaryl groups,
—NHheteroaryl groups,
—N(heteroaryl groups)₂,
—NHC(O)heteroaryl groups,
—C(O)NHheteroaryl groups,
C₁-C₆ linear, branched, and cyclic alkyl groups,
C₂-C₆ linear, branched, and cyclic alkenyl groups,
C₁-C₆ linear, branched, and cyclic hydroxyalkyl groups,
C₁-C₆ linear, branched, and cyclic alkoxy groups,
C₁-C₆ linear, branched, and cyclic thioalkyl groups,
C₁-C₆ linear, branched, and cyclic haloalkyl groups,
C₁-C₆ linear, branched, and cyclic haloaminoalkyl groups,
C₁-C₆ linear, branched, and cyclic halothioalkyl groups,
C₁-C₆ linear, branched, and cyclic haloalkoxy groups,
benzyloxy, benzylamino, and benzylthio groups,
3 to 6-membered heterocycloalkenyl groups,
3 to 6-membered heterocyclic groups, and
5 and 6-membered heteroaryl groups.

25. The compound, tautomer, deuterated derivative, or pharmaceutically acceptable salt of claim 23 or 24, wherein R¹ is chosen from cyclic alkyl groups, carbocyclic groups, heterocyclic groups, aryl groups, and heteroaryl groups; R² is an aryl group; and R³ is chosen from linear and branched alkyl groups.

26. The compound, tautomer, deuterated derivative, or pharmaceutically acceptable salt of claim 23 or 24, wherein R¹ is chosen from optionally substituted 5 and 6-membered aryl groups.

27. The compound, tautomer, deuterated derivative, or pharmaceutically acceptable salt of claim 26, wherein R¹ is chosen from 5 and 6-membered aryl groups substituted with at least one halogen group.

28. The compound, tautomer, deuterated derivative, or pharmaceutically acceptable salt of claim 27, wherein R¹ is chosen from 5 and 6-membered aryl groups substituted with at least one fluoro.

29. The compound, tautomer, deuterated derivative, or pharmaceutically acceptable salt of claim 27, wherein R¹ is chosen from 5 and 6-membered aryl groups substituted with at least one chloro.

30. The compound, tautomer, deuterated derivative, or pharmaceutically acceptable salt of claim 23 or 24, wherein R¹ is chosen from optionally substituted 5 and 6-membered heteroaryl groups.

31. The compound, tautomer, deuterated derivative, or pharmaceutically acceptable salt of claim 30, wherein R¹ is chosen from 5 and 6-membered heteroaryl groups substituted with at least one halogen group.

32. The compound, tautomer, deuterated derivative, or pharmaceutically acceptable salt of claim 31, wherein R¹ is chosen from 5 and 6-membered heteroaryl groups substituted with at least one fluoro.

33. The compound, tautomer, deuterated derivative, or pharmaceutically acceptable salt of claim 31, wherein R¹ is chosen from 5 and 6-membered heteroaryl groups substituted with at least one chloro.

34. The compound, tautomer, deuterated derivative, or pharmaceutically acceptable salt of claim 23 or 24, wherein R¹ is chosen from optionally substituted 3 to 6-membered cyclic alkyl groups.

35. The compound, tautomer, deuterated derivative, or pharmaceutically acceptable salt of claim 34, wherein R¹ is chosen from 3 to 6-membered cyclic alkyl groups substituted with at least one alkoxy group.

36. The compound, tautomer, deuterated derivative, or pharmaceutically acceptable salt of claim 35, wherein R¹ is chosen from 3 to 6-membered cyclic alkyl group substituted with at least one methoxy group.

37. The compound, tautomer, deuterated derivative, or pharmaceutically acceptable salt of claim 23 or 24, wherein R¹ is chosen from optionally substituted 3 to 6-membered carbocyclic or heterocyclic groups.

38. The compound, tautomer, deuterated derivative, or pharmaceutically acceptable salt of any one of claims 23 to 37, wherein R² is chosen from optionally substituted 5 and 6-membered heteroaryl groups.

39. The compound, tautomer, deuterated derivative, or pharmaceutically acceptable salt of any one of claims 23 to 38, wherein R² is chosen from 5 and 6-membered heteroaryl groups substituted with at least one halogen group.

40. The compound, tautomer, deuterated derivative, or pharmaceutically acceptable salt of claim 40, wherein R² is chosen from 5 and 6-membered heteroaryl groups substituted with at least one fluoro.

41. The compound, tautomer, deuterated derivative, or pharmaceutically acceptable salt of claim 40, wherein R² is chosen from 5 and 6-membered heteroaryl groups substituted with at least one chloro.

42. The compound, tautomer, deuterated derivative, or pharmaceutically acceptable salt of any one of claims 23 to 38, wherein R² is chosen from 5 and 6-membered heteroaryl groups substituted with at least one cyano.

43. The compound, tautomer, deuterated derivative, or pharmaceutically acceptable salt of any one of claims 23 to 38, wherein R² is chosen from 5 and 6-membered heteroaryl groups substituted with at least one group chosen from C₁-C₆ linear, C₃-C₆ branched, and C₃-C₆ cyclic alkyl groups.

44. The compound, tautomer, deuterated derivative, or pharmaceutically acceptable salt of any one of claims 23 to 43, wherein R³ is chosen from optionally substituted C₁-C₁₀ linear and C₂-C₁₀ branched alkyl groups.

45. The compound, tautomer, deuterated derivative, or pharmaceutically acceptable salt of claim 44, wherein R³ is chosen from methyl, ethyl, propyl, isopropyl, butyl, iso-butyl, sec-butyl, pentyl, iso-pentyl, sec-pentyl, neo-pentyl, and 1,2,2-trimethylpropyl.

46. The compound, tautomer, deuterated derivative, or pharmaceutically acceptable salt of any one of claims 23 to 45, wherein R⁴ is chosen from optionally substituted C₁-C₁₀ linear, C₂-C₁₀ branched alkyl groups, and C₃-C₆ cyclic alkyl groups.

47. The compound, tautomer, deuterated derivative, or pharmaceutically acceptable salt of claim 45, wherein R⁴ is chosen from methyl, ethyl, propyl, isopropyl, cyclopropyl, butyl, iso-butyl, sec-butyl, cyclobutyl, pentyl, iso-pentyl, sec-pentyl, neo-pentyl, 1,2,2-trimethylpropyl, cyclopentyl, and cyclohexyl.

48. A compound of Formula (IIIA): 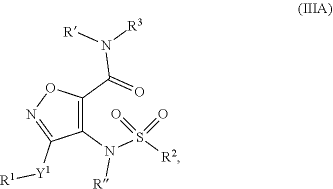
a tautomer thereof, a deuterated derivative of the compound or the tautomer, or a pharmaceutically acceptable salt of the foregoing, wherein:
(i) Y¹ is absent or is chosen from a bond, O, S, and NR⁵;
wherein R⁵ is chosen from hydrogen, linear, branched, and cyclic alkyl groups, carbocyclic groups, heterocyclic groups, aryl groups, and heteroaryl groups;
(ii) R1 is chosen from linear, branched, and cyclic alkyl groups, alkenyl groups, carbocyclic groups, heterocyclic groups, linear, branched, and cyclic alkenyl groups, linear and branched heteroalkenyl groups, aryl groups, and heteroaryl groups;
(iii) each R² and R³ is independently chosen from linear, branched, and cyclic alkyl groups, carbocyclic groups, heterocyclic groups, aryl groups, and heteroaryl groups;
(iv) each R′ and R″ is independently chosen from hydrogen, linear, branched, and cyclic alkyl groups, carbocyclic groups, heterocyclic groups, aryl groups, and heteroaryl groups;
wherein the linear, branched, and cyclic alkyl groups, linear, branched, and cyclic alkenyl groups, linear and branched heteroalkenyl groups, carbocyclic groups, heterocyclic groups, aryl groups, and heteroaryl groups are optionally substituted with at least one group chosen from the following groups:
halogen groups,
hydroxy,
thiol,
amino,
cyano,
—OC(O)C₁-C₆ linear, branched, and cyclic alkyl groups,
—C(O)OC₁-C₆ linear, branched, and cyclic alkyl groups,
—NHC₁-C₆ linear, branched, and cyclic alkyl groups,
—N(C₁-C₆ linear, branched, and cyclic alkyl groups)₂,
—NHC(O)C₁-C₆ linear, branched, and cyclic alkyl groups,
—C(O)NHC₁-C₆ linear, branched, and cyclic alkyl groups,
—NHaryl groups,
—N(aryl groups)₂,
—NHC(O)aryl groups,
—C(O)NHaryl groups,
—NHheteroaryl groups,
—N(heteroaryl groups)₂,
—NHC(O)heteroaryl groups,
—C(O)NHheteroaryl groups,
C₁-C₆ linear, branched, and cyclic alkyl groups,
C₂-C₆ linear, branched, and cyclic alkenyl groups,
C₁-C₆ linear, branched, and cyclic hydroxyalkyl groups,
C₁-C₆ linear, branched, and cyclic alkoxy groups,
C₁-C₆ linear, branched, and cyclic thioalkyl groups,
C₁-C₆ linear, branched, and cyclic haloalkyl groups,
C₁-C₆ linear, branched, and cyclic haloaminoalkyl groups,
C₁-C₆ linear, branched, and cyclic halothioalkyl groups,
C₁-C₆ linear, branched, and cyclic haloalkoxy groups,
benzyloxy, benzylamino, and benzylthio groups,
3 to 6-membered heterocycloalkenyl groups,
3 to 6-membered heterocyclic groups, and
5 and 6-membered heteroaryl groups.

49. A compound of Formula (IIIB): 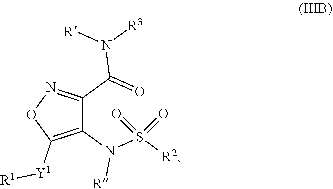
a tautomer thereof, a deuterated derivative of the compound or the tautomer, or a pharmaceutically acceptable salt of the foregoing, wherein:
(i) Y¹ is absent or is chosen from a bond, O, S, and NR⁵;
wherein R⁵ is chosen from hydrogen, linear, branched, and cyclic alkyl groups, carbocyclic groups, heterocyclic groups, aryl groups, and heteroaryl groups;
(ii) R¹ is chosen from linear, branched, and cyclic alkyl groups, alkenyl groups, carbocyclic groups, heterocyclic groups, linear, branched, and cyclic alkenyl groups, linear and branched heteroalkenyl groups, aryl groups, and heteroaryl groups;
(iii) each R² and R³ is independently chosen from linear, branched, and cyclic alkyl groups, carbocyclic groups, heterocyclic groups, aryl groups, and heteroaryl groups;
(iv) each R′ and R″ is independently chosen from hydrogen, linear, branched, and cyclic alkyl groups, carbocyclic groups, heterocyclic groups, aryl groups, and heteroaryl groups;
wherein the linear, branched, and cyclic alkyl groups, linear, branched, and cyclic alkenyl groups, linear and branched heteroalkenyl groups, carbocyclic groups, heterocyclic groups, aryl groups, and heteroaryl groups are optionally substituted with at least one group chosen from the following groups:
halogen groups,
hydroxy,
thiol,
amino,
cyano,
—OC(O)C₁-C₆ linear, branched, and cyclic alkyl groups,
—C(O)OC₁-C₆ linear, branched, and cyclic alkyl groups,
—NHC₁-C₆ linear, branched, and cyclic alkyl groups,
—N(C₁-C₆ linear, branched, and cyclic alkyl groups)₂,
—NHC(O)C₁-C₆ linear, branched, and cyclic alkyl groups,
—C(O)NHC₁-C₆ linear, branched, and cyclic alkyl groups,
—NHaryl groups,
—N(aryl groups)₂,
—NHC(O)aryl groups,
—C(O)NHaryl groups,
—NHheteroaryl groups,
—N(heteroaryl groups)₂,
—NHC(O)heteroaryl groups,
—C(O)NHheteroaryl groups,
C₁-C₆ linear, branched, and cyclic alkyl groups,
C₂-C₆ linear, branched, and cyclic alkenyl groups,
C₁-C₆ linear, branched, and cyclic hydroxyalkyl groups,
C₁-C₆ linear, branched, and cyclic alkoxy groups,
C₁-C₆ linear, branched, and cyclic thioalkyl groups,
C₁-C₆ linear, branched, and cyclic haloalkyl groups,
C₁-C₆ linear, branched, and cyclic haloaminoalkyl groups,
C₁-C₆ linear, branched, and cyclic halothioalkyl groups,
C₁-C₆ linear, branched, and cyclic haloalkoxy groups,
benzyloxy, benzylamino, and benzylthio groups,
3 to 6-membered heterocycloalkenyl groups,
3 to 6-membered heterocyclic groups, and
5 and 6-membered heteroaryl groups.

50. The compound, tautomer, deuterated derivative, or pharmaceutically acceptable salt of claim 48 or 49, wherein R¹ is chosen from cyclic alkyl groups, carbocyclic groups, heterocyclic groups, aryl groups, and heteroaryl groups; R² is an aryl group; and R³ is chosen from linear and branched alkyl groups.

51. The compound, tautomer, deuterated derivative, or pharmaceutically acceptable salt of claim 48 or 49, wherein R¹ is chosen from optionally substituted 5 and 6-membered aryl groups.

52. The compound, tautomer, deuterated derivative, or pharmaceutically acceptable salt of claim 51, wherein R¹ is chosen from 5 and 6-membered aryl groups substituted with at least one halogen group.

53. The compound, tautomer, deuterated derivative, or pharmaceutically acceptable salt of claim 52, wherein R¹ is chosen from 5 and 6-membered aryl groups substituted with at least one fluoro.

54. The compound, tautomer, deuterated derivative, or pharmaceutically acceptable salt of claim 52, wherein R¹ is chosen from 5 and 6-membered aryl groups substituted with at least one chloro.

55. The compound, tautomer, deuterated derivative, or pharmaceutically acceptable salt of claim 48 or 49, wherein R¹ is chosen from optionally substituted 5 and 6-membered heteroaryl groups.

56. The compound, tautomer, deuterated derivative, or pharmaceutically acceptable salt of claim 55, wherein R¹ is chosen from 5 and 6-membered heteroaryl groups substituted with at least one halogen group.

57. The compound, tautomer, deuterated derivative, or pharmaceutically acceptable salt of claim 56, wherein R¹ is chosen from 5 and 6-membered heteroaryl groups substituted with at least one fluoro.

58. The compound, tautomer, deuterated derivative, or pharmaceutically acceptable salt of claim 56, wherein R¹ is chosen from 5 and 6-membered heteroaryl groups substituted with at least one chloro.

59. The compound, tautomer, deuterated derivative, or pharmaceutically acceptable salt of claim 48 or 49, wherein R¹ is chosen from optionally substituted 3 to 6-membered cyclic alkyl groups.

60. The compound, tautomer, deuterated derivative, or pharmaceutically acceptable salt of claim 59, wherein R¹ is chosen from 3 to 6-membered cyclic alkyl groups substituted with at least one alkoxy group.

61. The compound, tautomer, deuterated derivative, or pharmaceutically acceptable salt of claim 60, wherein R¹ is chosen from 3 to 6-membered cyclic alkyl groups substituted with at least one methoxy group.

62. The compound, tautomer, deuterated derivative, or pharmaceutically acceptable salt of claim 48 or 49, wherein R¹ is chosen from optionally substituted 3 to 6-membered carbocyclic or heterocyclic groups.

63. The compound, tautomer, deuterated derivative, or pharmaceutically acceptable salt of any one of claims 48 to 62, wherein R² is chosen from optionally substituted 5 and 6-membered heteroaryl groups.

64. The compound, tautomer, deuterated derivative, or pharmaceutically acceptable salt of any one of claims 48 to 63, wherein R² is chosen from 5 and 6-membered heteroaryl groups substituted with at least one halogen group.

65. The compound, tautomer, deuterated derivative, or pharmaceutically acceptable salt of claim 64, wherein R² is chosen from 5 and 6-membered heteroaryl groups substituted with at least one fluoro.

66. The compound, tautomer, deuterated derivative, or pharmaceutically acceptable salt of claim 64, wherein R² is chosen from 5 and 6-membered heteroaryl groups substituted with at least one chloro.

67. The compound, tautomer, deuterated derivative, or pharmaceutically acceptable salt of any one of claims 48 to 62, wherein R² is chosen from 5 and 6-membered heteroaryl groups substituted with at least one cyano.

68. The compound, tautomer, deuterated derivative, or pharmaceutically acceptable salt of any one of claims 48 to 62, wherein R² is chosen from 5 and 6-membered heteroaryl groups substituted with at least one group chosen from C₁-C₆ linear, C₃-C₆ branched, and C₃-C₆ cyclic alkyl groups.

69. The compound, tautomer, deuterated derivative, or pharmaceutically acceptable salt of any one of claims 48 to 68, wherein R³ is chosen from optionally substituted C₁-C₁₀ linear and C₂-C₁₀ branched alkyl groups.

70. The compound, tautomer, deuterated derivative, or pharmaceutically acceptable salt of claim 69, wherein R³ is chosen from methyl, ethyl, propyl, isopropyl, butyl, iso-butyl, sec-butyl, pentyl, iso-pentyl, sec-pentyl, neo-pentyl, and 1,2,2-trimethylpropyl.

71. The compound, tautomer, deuterated derivative, or pharmaceutically acceptable salt of any one of claims 48 to 70, wherein R⁴ is chosen from optionally substituted C₁-C₁₀ linear, C₂-C₁₀ branched alkyl groups, and C₃-C₆ cyclic alkyl groups.

72. The compound, tautomer, deuterated derivative, or pharmaceutically acceptable salt of claim 71, wherein R⁴ is chosen from methyl, ethyl, propyl, isopropyl, cyclopropyl, butyl, iso-butyl, sec-butyl, cyclobutyl, pentyl, iso-pentyl, sec-pentyl, neo-pentyl, 1,2,2-trimethylpropyl, cyclopentyl, and cyclohexyl.

73. A compound of Formula (IVA): 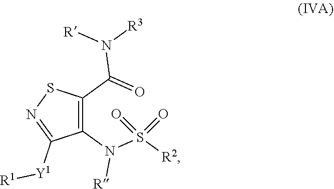
a tautomer thereof, a deuterated derivative of the compound or the tautomer, or a pharmaceutically acceptable salt of the foregoing, wherein:
(i) Y¹ is absent or is chosen from a bond, O, S, and NR⁵;
wherein R⁵ is chosen from hydrogen, linear, branched, and cyclic alkyl groups, carbocyclic groups, heterocyclic groups, aryl groups, and heteroaryl groups;
(ii) R¹ is chosen from linear, branched, and cyclic alkyl groups, alkenyl groups, carbocyclic groups, heterocyclic groups, linear, branched, and cyclic alkenyl groups, linear and branched heteroalkenyl groups, aryl groups, and heteroaryl groups;
(iii) each R² and R³ is independently chosen from linear, branched, and cyclic alkyl groups, carbocyclic groups, heterocyclic groups, aryl groups, and heteroaryl groups;
(iv) each R′ and R″ is independently chosen from hydrogen, linear, branched, and cyclic alkyl groups, heterocyclic groups, aryl groups, and heteroaryl groups;
wherein the linear, branched, and cyclic alkyl groups, linear, branched, and cyclic alkenyl groups, linear and branched heteroalkenyl groups, carbocyclic groups, heterocyclic groups, aryl groups, and heteroaryl groups are optionally substituted with at least one group chosen from the following groups:
halogen groups,
hydroxy,
thiol,
amino,
cyano,
—OC(O)C₁-C₆ linear, branched, and cyclic alkyl groups,
—C(O)OC₁-C₆ linear, branched, and cyclic alkyl groups,
—NHC₁-C₆ linear, branched, and cyclic alkyl groups,
—N(C₁-C₆ linear, branched, and cyclic alkyl groups)₂,
—NHC(O)C₁-C₆ linear, branched, and cyclic alkyl groups,
—C(O)NHC₁-C₆ linear, branched, and cyclic alkyl groups,
—NHaryl groups,
—N(aryl groups)₂,
—NHC(O)aryl groups,
—C(O)NHaryl groups,
—NHheteroaryl groups,
—N(heteroaryl groups)₂,
—NHC(O)heteroaryl groups,
—C(O)NHheteroaryl groups,
C₁-C₆ linear, branched, and cyclic alkyl groups,
C₂-C₆ linear, branched, and cyclic alkenyl groups,
C₁-C₆ linear, branched, and cyclic hydroxyalkyl groups,
C₁-C₆ linear, branched, and cyclic alkoxy groups,
C₁-C₆ linear, branched, and cyclic thioalkyl groups,
C₁-C₆ linear, branched, and cyclic haloalkyl groups,
C₁-C₆ linear, branched, and cyclic haloaminoalkyl groups,
C₁-C₆ linear, branched, and cyclic halothioalkyl groups,
C₁-C₆ linear, branched, and cyclic haloalkoxy groups,
benzyloxy, benzylamino, and benzylthio groups,
3 to 6-membered heterocycloalkenyl groups,
3 to 6-membered heterocyclic groups, and
5 and 6-membered heteroaryl groups.

74. A compound of Formula (IVB): 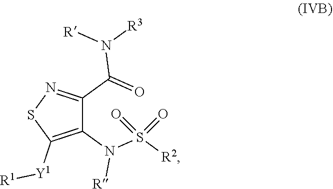
a tautomer thereof, a deuterated derivative of the compound or the tautomer, or a pharmaceutically acceptable salt of the foregoing, wherein:
(i) Y¹ is absent or is chosen from a bond, O, S, and NR⁵;
wherein R⁵ is chosen from hydrogen, linear, branched, and cyclic alkyl groups, carbocyclic groups, heterocyclic groups, aryl groups, and heteroaryl groups;
(ii) R¹ is chosen from linear, branched, and cyclic alkyl groups, alkenyl groups, carbocyclic groups, heterocyclic groups, linear, branched, and cyclic alkenyl groups, linear and branched heteroalkenyl groups, aryl groups, and heteroaryl groups;
(iii) each R² and R³ is independently chosen from linear, branched, and cyclic alkyl groups, carbocyclic groups, heterocyclic groups, aryl groups, and heteroaryl groups;
(iv) each R′ and R″ is independently chosen from hydrogen, linear, branched, and cyclic alkyl groups, carbocyclic groups, heterocyclic groups, aryl groups, and heteroaryl groups;
wherein the linear, branched, and cyclic alkyl groups, linear, branched, and cyclic alkenyl groups, linear and branched heteroalkenyl groups, carbocyclic groups, heterocyclic groups, aryl groups, and heteroaryl groups are optionally substituted with at least one group chosen from the following groups:
halogen groups,
hydroxy,
thiol,
amino,
cyano,
—OC(O)C₁-C₆ linear, branched, and cyclic alkyl groups,
—C(O)OC₁-C₆ linear, branched, and cyclic alkyl groups,
—NHC₁-C₆ linear, branched, and cyclic alkyl groups,
—N(C₁-C₆ linear, branched, and cyclic alkyl groups)₂,
—NHC(O)C₁-C₆ linear, branched, and cyclic alkyl groups,
—C(O)NHC₁-C₆ linear, branched, and cyclic alkyl groups,
—NHaryl groups,
—N(aryl groups)₂,
—NHC(O)aryl groups,
—C(O)NHaryl groups,
—NHheteroaryl groups,
—N(heteroaryl groups)₂,
—NHC(O)heteroaryl groups,
—C(O)NHheteroaryl groups,
C₁-C₆ linear, branched, and cyclic alkyl groups,
C₂-C₆ linear, branched, and cyclic alkenyl groups,
C₁-C₆ linear, branched, and cyclic hydroxyalkyl groups,
C₁-C₆ linear, branched, and cyclic alkoxy groups,
C₁-C₆ linear, branched, and cyclic thioalkyl groups,
C₁-C₆ linear, branched, and cyclic haloalkyl groups,
C₁-C₆ linear, branched, and cyclic haloaminoalkyl groups,
C₁-C₆ linear, branched, and cyclic halothioalkyl groups,
C₁-C₆ linear, branched, and cyclic haloalkoxy groups,
benzyloxy, benzylamino, and benzylthio groups,
3 to 6-membered heterocycloalkenyl groups,
3 to 6-membered heterocyclic groups, and
5 and 6-membered heteroaryl groups.

75. The compound, tautomer, deuterated derivative, or pharmaceutically acceptable salt of claim 73 or 74, wherein R¹ is chosen from cyclic alkyl groups, carbocyclic groups, heterocyclic groups, aryl groups, and heteroaryl groups; R² is an aryl group; and R³ is chosen from linear and branched, alkyl groups.

76. The compound, tautomer, deuterated derivative, or pharmaceutically acceptable salt of claim 73 or 74, wherein R¹ is chosen from optionally substituted 5 and 6-membered aryl groups.

77. The compound, tautomer, deuterated derivative, or pharmaceutically acceptable salt of claim 76, wherein R¹ is chosen from 5 and 6-membered aryl groups substituted with at least one halogen group.

78. The compound, tautomer, deuterated derivative, or pharmaceutically acceptable salt of claim 77, wherein R¹ is chosen from 5 and 6-membered aryl groups substituted with at least one fluoro.

79. The compound, tautomer, deuterated derivative, or pharmaceutically acceptable salt of claim 77, wherein R¹ is chosen from 5 and 6-membered aryl groups substituted with at least one chloro.

80. The compound, tautomer, deuterated derivative, or pharmaceutically acceptable salt of claim 73 or 74, wherein R¹ is chosen from optionally substituted 5 and 6-membered heteroaryl groups.

81. The compound, tautomer, deuterated derivative, or pharmaceutically acceptable salt of claim 80, wherein R¹ is chosen from 5 and 6-membered heteroaryl groups substituted with at least one halogen group.

82. The compound, tautomer, deuterated derivative, or pharmaceutically acceptable salt of claim 81, wherein R¹ is chosen from 5 and 6-membered heteroaryl groups substituted with at least one fluoro.

83. The compound, tautomer, deuterated derivative, or pharmaceutically acceptable salt of claim 81, wherein R¹ is chosen from 5 and 6-membered heteroaryl groups substituted with at least one chloro.

84. The compound, tautomer, deuterated derivative, or pharmaceutically acceptable salt of claim 73 or 74, wherein R¹ is chosen from optionally substituted 3 to 6-membered cyclic alkyl groups.

85. The compound, tautomer, deuterated derivative, or pharmaceutically acceptable salt of claim 84, wherein R¹ is chosen from 3 to 6-membered cyclic alkyl groups substituted with at least one alkoxy group.

86. The compound, tautomer, deuterated derivative, or pharmaceutically acceptable salt of claim 85, wherein R¹ is chosen from 3 to 6-membered cyclic alkyl groups substituted with at least one methoxy group.

87. The compound, tautomer, deuterated derivative, or pharmaceutically acceptable salt of claim 73 or 74, wherein R¹ is chosen from optionally substituted 3 to 6-membered carbocyclic or heterocyclic groups.

88. The compound, tautomer, deuterated derivative, or pharmaceutically acceptable salt of any one of claims 73 to 87, wherein R² is chosen from optionally substituted 5 and 6-membered heteroaryl groups.

89. The compound, tautomer, deuterated derivative, or pharmaceutically acceptable salt of any one of claims 73 to 88, wherein R² is chosen from 5 and 6-membered heteroaryl groups substituted with at least one halogen group.

90. The compound, tautomer, deuterated derivative, or pharmaceutically acceptable salt of claim 89, wherein R² is chosen from 5 and 6-membered heteroaryl groups substituted with at least one fluoro.

91. The compound, tautomer, deuterated derivative, or pharmaceutically acceptable salt of claim 89, wherein R² is chosen from 5 and 6-membered heteroaryl groups substituted with at least one chloro.

92. The compound, tautomer, deuterated derivative, or pharmaceutically acceptable salt of any one of claims 73 to 87, wherein R² is chosen from 5 and 6-membered heteroaryl groups substituted with at least one cyano.

93. The compound, tautomer, deuterated derivative, or pharmaceutically acceptable salt of any one of claims 73 to 87, wherein R² is chosen from 5 and 6-membered heteroaryl groups substituted with at least one group chosen from C₁-C₆ linear, C₃-C₆ branched, and C₃-C₆ cyclic alkyl groups.

94. The compound, tautomer, deuterated derivative, or pharmaceutically acceptable salt of any one of claims 73 to 93, wherein R³ is chosen from optionally substituted C₁-C₁₀ linear and C₂-C₁₀ branched alkyl groups.

95. The compound, tautomer, deuterated derivative, or pharmaceutically acceptable salt of claim 94, wherein R³ is chosen from methyl, ethyl, propyl, isopropyl, butyl, iso-butyl, sec-butyl, pentyl, iso-pentyl, sec-pentyl, neo-pentyl, and 1,2,2-trimethylpropyl.

96. A compound of Formula (VA): 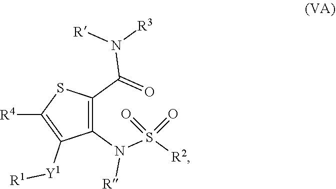
a tautomer thereof, a deuterated derivative of the compound or the tautomer, or a pharmaceutically acceptable salt of the foregoing, wherein:
(i) Y¹ is absent or is chosen from a bond, O, S, and NR⁵;
wherein R⁵ is chosen from hydrogen, linear, branched, and cyclic alkyl groups, carbocyclic groups, heterocyclic groups, aryl groups, and heteroaryl groups;
(ii) R¹ is chosen from linear, branched, and cyclic alkyl groups, alkenyl groups, heterocyclic groups, linear, branched, and cyclic alkenyl groups, linear and branched heteroalkenyl groups, aryl groups, and heteroaryl groups;
(iii) each R² and R³ is independently chosen from linear, branched, and cyclic alkyl groups, carbocyclic groups, heterocyclic groups, aryl groups, and heteroaryl groups;
(iv) each R′ and R″ is independently chosen from hydrogen, linear, branched, and cyclic alkyl groups, carbocyclic groups, heterocyclic groups, aryl groups, and heteroaryl groups;
wherein the linear, branched, and cyclic alkyl groups, linear, branched, and cyclic alkenyl groups, linear and branched heteroalkenyl groups, carbocyclic groups, heterocyclic groups, aryl groups, and heteroaryl groups are optionally substituted with at least one group chosen from the following groups:
halogen groups,
hydroxy,
thiol,
amino,
cyano,
—OC(O)C₁-C₆ linear, branched, and cyclic alkyl groups,
—C(O)OC₁-C₆ linear, branched, and cyclic alkyl groups,
—NHC₁-C₆ linear, branched, and cyclic alkyl groups,
—N(C₁-C₆ linear, branched, and cyclic alkyl groups)₂,
—NHC(O)C₁-C₆ linear, branched, and cyclic alkyl groups,
—C(O)NHC₁-C₆ linear, branched, and cyclic alkyl groups,
—NHaryl groups,
—N(aryl groups)₂,
—NHC(O)aryl groups,
—C(O)NHaryl groups,
—NHheteroaryl groups,
—N(heteroaryl groups)₂,
—NHC(O)heteroaryl groups,
—C(O)NHheteroaryl groups,
C₁-C₆ linear, branched, and cyclic alkyl groups,
C₂-C₆ linear, branched, and cyclic alkenyl groups,
C₁-C₆ linear, branched, and cyclic hydroxyalkyl groups,
C₁-C₆ linear, branched, and cyclic alkoxy groups,
C₁-C₆ linear, branched, and cyclic thioalkyl groups,
C₁-C₆ linear, branched, and cyclic haloalkyl groups,
C₁-C₆ linear, branched, and cyclic haloaminoalkyl groups,
C₁-C₆ linear, branched, and cyclic halothioalkyl groups,
C₁-C₆ linear, branched, and cyclic haloalkoxy groups,
benzyloxy, benzylamino, and benzylthio groups,
3 to 6-membered heterocycloalkenyl groups,
3 to 6-membered heterocyclic groups, and
5 and 6-membered heteroaryl groups.

97. A compound of Formula (VB): 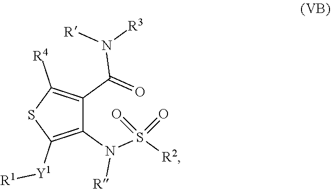
a tautomer thereof, a deuterated derivative of the compound or the tautomer, or a pharmaceutically acceptable salt of the foregoing, wherein:
(i) Y¹ is absent or is chosen from a bond, O, S, and NR⁵;
wherein R⁵ is chosen from hydrogen, linear, branched, and cyclic alkyl groups, heterocyclic groups, aryl groups, and heteroaryl groups;
(ii) R¹ is chosen from linear, branched, and cyclic alkyl groups, alkenyl groups, carbocyclic groups, heterocyclic groups, linear, branched, and cyclic alkenyl groups, linear and branched heteroalkenyl groups, aryl groups, and heteroaryl groups;
(iii) each R² and R³ is independently chosen from linear, branched, and cyclic alkyl groups, carbocyclic groups, heterocyclic groups, aryl groups, and heteroaryl groups;
(iv) each R′ and R″ is independently chosen from hydrogen, linear, branched, and cyclic alkyl groups, heterocyclic groups, aryl groups, and heteroaryl groups;
wherein the linear, branched, and cyclic alkyl groups, linear, branched, and cyclic alkenyl groups, linear and branched heteroalkenyl groups, carbocyclic groups, heterocyclic groups, aryl groups, and heteroaryl groups are optionally substituted with at least one group chosen from the following groups:
halogen groups,
hydroxy,
thiol,
amino,
cyano,
—OC(O)C₁-C₆ linear, branched, and cyclic alkyl groups,
—C(O)OC₁-C₆ linear, branched, and cyclic alkyl groups,
—NHC₁-C₆ linear, branched, and cyclic alkyl groups,
—N(C₁-C₆ linear, branched, and cyclic alkyl groups)₂,
—NHC(O)C₁-C₆ linear, branched, and cyclic alkyl groups,
—C(O)NHC₁-C₆ linear, branched, and cyclic alkyl groups,
—NHaryl groups,
—N(aryl groups)₂,
—NHC(O)aryl groups,
—C(O)NHaryl groups,
—NHheteroaryl groups,
—N(heteroaryl groups)₂,
—NHC(O)heteroaryl groups,
—C(O)NHheteroaryl groups,
C₁-C₆ linear, branched, and cyclic alkyl groups,
C₂-C₆ linear, branched, and cyclic alkenyl groups,
C₁-C₆ linear, branched, and cyclic hydroxyalkyl groups,
C₁-C₆ linear, branched, and cyclic alkoxy groups,
C₁-C₆ linear, branched, and cyclic thioalkyl groups,
C₁-C₆ linear, branched, and cyclic haloalkyl groups,
C₁-C₆ linear, branched, and cyclic haloaminoalkyl groups,
C₁-C₆ linear, branched, and cyclic halothioalkyl groups,
C₁-C₆ linear, branched, and cyclic haloalkoxy groups,
benzyloxy, benzylamino, and benzylthio groups,
3 to 6-membered heterocycloalkenyl groups,
3 to 6-membered heterocyclic groups, and
5 and 6-membered heteroaryl groups.

98. The compound, tautomer, deuterated derivative, or pharmaceutically acceptable salt of claim 96 or 97, wherein R¹ is chosen from cyclic alkyl groups, carbocyclic groups, heterocyclic groups, aryl groups, and heteroaryl groups; R² is an aryl group; and R³ is chosen from linear or branched alkyl groups.

99. The compound, tautomer, deuterated derivative, or pharmaceutically acceptable salt of claim 96 or 97, wherein R¹ is chosen from optionally substituted 5 and 6-membered aryl groups.

100. The compound, tautomer, deuterated derivative, or pharmaceutically acceptable salt of claim 99, wherein R¹ is chosen from 5 and 6-membered aryl groups substituted with at least one halogen group.

101. The compound, tautomer, deuterated derivative, or pharmaceutically acceptable salt of claim 100, wherein R¹ is chosen from 5 and 6-membered aryl groups substituted with at least one fluoro.

102. The compound, tautomer, deuterated derivative, or pharmaceutically acceptable salt of claim 100, wherein R¹ is chosen from 5 and 6-membered aryl groups substituted with at least one chloro.

103. The compound, tautomer, deuterated derivative, or pharmaceutically acceptable salt of claim 96 or 97, wherein R¹ is chosen from optionally substituted 5 and 6-membered heteroaryl groups.

104. The compound, tautomer, deuterated derivative, or pharmaceutically acceptable salt of claim 103, wherein R¹ is chosen from 5 and 6-membered heteroaryl groups substituted with at least one halogen group.

105. The compound, tautomer, deuterated derivative, or pharmaceutically acceptable salt of claim 104, wherein R¹ is chosen from 5 and 6-membered heteroaryl groups substituted with at least one fluoro.

106. The compound, tautomer, deuterated derivative, or pharmaceutically acceptable salt of claim 104, wherein R¹ is chosen from 5 and 6-membered heteroaryl groups substituted with at least one chloro.

107. The compound, tautomer, deuterated derivative, or pharmaceutically acceptable salt of claim 96 or 97, wherein R¹ is chosen from optionally substituted 3 to 6-membered cyclic alkyl groups.

108. The compound, tautomer, deuterated derivative, or pharmaceutically acceptable salt of claim 107, wherein R¹ is chosen from 3 to 6-membered cyclic alkyl groups substituted with at least one alkoxy group.

109. The compound, tautomer, deuterated derivative, or pharmaceutically acceptable salt of claim 108, wherein R¹ is chosen from 3 to 6-membered cyclic alkyl groups substituted with at least one methoxy group.

110. The compound, tautomer, deuterated derivative, or pharmaceutically acceptable salt of claim 96 or 97, wherein R¹ is chosen from optionally substituted 3 to 6-membered carbocyclic or heterocyclic groups.

111. The compound, tautomer, deuterated derivative, or pharmaceutically acceptable salt of any one of claims 96 to 110, wherein R² is chosen from optionally substituted 5 and 6-membered heteroaryl groups.

112. The compound, tautomer, deuterated derivative, or pharmaceutically acceptable salt of any one of claims 96 to 111, wherein R² is chosen from 5 and 6-membered heteroaryl groups substituted with at least one halogen group.

113. The compound, tautomer, deuterated derivative, or pharmaceutically acceptable salt of claim 112, wherein R² is chosen from 5 and 6-membered heteroaryl groups substituted with at least one fluoro.

114. The compound, tautomer, deuterated derivative, or pharmaceutically acceptable salt of claim 112, wherein R² is chosen from 5 and 6-membered heteroaryl groups substituted with at least one chloro.

115. The compound, tautomer, deuterated derivative, or pharmaceutically acceptable salt of any one of claims 96 to 110, wherein R² is chosen from 5 and 6-membered heteroaryl groups substituted with at least one cyano.

116. The compound, tautomer, deuterated derivative, or pharmaceutically acceptable salt of any one of claims 96 to 110, wherein R² is chosen from 5 and 6-membered heteroaryl groups substituted with at least one group chosen from C₁-C₆ linear, C₃-C₆ branched, and C₃-C₆ cyclic alkyl groups.

117. The compound, tautomer, deuterated derivative, or pharmaceutically acceptable salt of any one of claims 96 to 116, wherein R³ is chosen from optionally substituted C₁-C₁₀ linear and C₂-C₁₀ branched alkyl groups.

118. The compound, tautomer, deuterated derivative, or pharmaceutically acceptable salt of claim 117, wherein R³ is chosen from methyl, ethyl, propyl, isopropyl, butyl, iso-butyl, sec-butyl, pentyl, iso-pentyl, sec-pentyl, neo-pentyl, and 1,2,2-trimethylpropyl.

119. The compound, tautomer, deuterated derivative, or pharmaceutically acceptable salt of any one of claims 96 to 118, wherein R⁴ is chosen from optionally substituted C₁-C₁₀ linear, C₂-C₁₀ branched alkyl groups, and C₃-C₆ cyclic alkyl groups.

120. The compound, tautomer, deuterated derivative, or pharmaceutically acceptable salt of claim 119, wherein R⁴ is chosen from methyl, ethyl, propyl, isopropyl, cyclopropyl, butyl, iso-butyl, sec-butyl, cyclobutyl, pentyl, iso-pentyl, sec-pentyl, neo-pentyl, 1,2,2-trimethylpropyl, cyclopentyl, and cyclohexyl.

121. A compound of Formula (VIA): 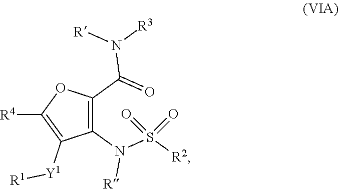
a tautomer thereof, a deuterated derivative of the compound or the tautomer, or a pharmaceutically acceptable salt of the foregoing, wherein:
(i) Y¹ is absent or is chosen from a bond, O, S, and NR⁵;
wherein R⁵ is chosen from hydrogen, linear, branched, and cyclic alkyl groups, carbocyclic groups, heterocyclic groups, aryl groups, and heteroaryl groups;
(ii) R¹ is chosen from linear, branched, and cyclic alkyl groups, alkenyl groups, carbocyclic groups, heterocyclic groups, linear, branched, and cyclic alkenyl groups, linear and branched heteroalkenyl groups, aryl groups, and heteroaryl groups;
(iii) each R² and R³ is independently chosen from linear, branched, and cyclic alkyl groups, carbocyclic groups, heterocyclic groups, aryl groups, and heteroaryl groups;
(iv) each R′ and R″ is independently chosen from hydrogen, linear, branched, and cyclic alkyl groups, carbocyclic groups, heterocyclic groups, aryl groups, and heteroaryl groups;
wherein the linear, branched, and cyclic alkyl groups, linear, branched, and cyclic alkenyl groups, linear and branched heteroalkenyl groups, carbocyclic groups, heterocyclic groups, aryl groups, and heteroaryl groups are optionally substituted with at least one group chosen from the following groups:
halogen groups,
hydroxy,
thiol,
amino,
cyano,
—OC(O)C₁-C₆ linear, branched, and cyclic alkyl groups,
—C(O)OC₁-C₆ linear, branched, and cyclic alkyl groups,
—NHC₁-C₆ linear, branched, and cyclic alkyl groups,
—N(C₁-C₆ linear, branched, and cyclic alkyl groups)₂,
—NHC(O)C₁-C₆ linear, branched, and cyclic alkyl groups,
—C(O)NHC₁-C₆ linear, branched, and cyclic alkyl groups,
—NHaryl groups,
—N(aryl groups)₂,
—NHC(O)aryl groups,
—C(O)NHaryl groups,
—NHheteroaryl groups,
—N(heteroaryl groups)₂,
—NHC(O)heteroaryl groups,
—C(O)NHheteroaryl groups,
C₁-C₆ linear, branched, and cyclic alkyl groups,
C₂-C₆ linear, branched, and cyclic alkenyl groups,
C₁-C₆ linear, branched, and cyclic hydroxyalkyl groups,
C₁-C₆ linear, branched, and cyclic alkoxy groups,
C₁-C₆ linear, branched, and cyclic thioalkyl groups,
C₁-C₆ linear, branched, and cyclic haloalkyl groups,
C₁-C₆ linear, branched, and cyclic haloaminoalkyl groups,
C₁-C₆ linear, branched, and cyclic halothioalkyl groups,
C₁-C₆ linear, branched, and cyclic haloalkoxy groups,
benzyloxy, benzylamino, and benzylthio groups,
3 to 6-membered heterocycloalkenyl groups,
3 to 6-membered heterocyclic groups, and
5 and 6-membered heteroaryl groups.

122. A compound of Formula (VIB): 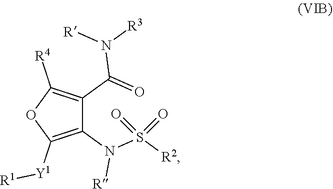
a tautomer thereof, a deuterated derivative of the compound or the tautomer, or a pharmaceutically acceptable salt of the foregoing, wherein:
(i) Y¹ is absent or is chosen from a bond, O, S, and NR⁵;
wherein R⁵ is chosen from hydrogen, linear, branched, and cyclic alkyl groups, carbocyclic groups, heterocyclic groups, aryl groups, and heteroaryl groups;
(ii) R¹ is chosen from linear, branched, and cyclic alkyl groups, alkenyl groups, carbocyclic groups, heterocyclic groups, linear, branched, and cyclic alkenyl groups, linear and branched heteroalkenyl groups, aryl groups, and heteroaryl groups;
(iii) each R² and R³ is independently chosen from linear, branched, and cyclic alkyl groups, carbocyclic groups, heterocyclic groups, aryl groups, and heteroaryl groups;
(iv) each R′ and R″ is independently chosen from hydrogen, linear, branched, and cyclic alkyl groups, carbocyclic groups, heterocyclic groups, aryl groups, and heteroaryl groups;
wherein the linear, branched, and cyclic alkyl groups, linear, branched, and cyclic alkenyl groups, linear and branched heteroalkenyl groups, carbocyclic groups, heterocyclic groups, aryl groups, and heteroaryl groups are optionally substituted with at least one group chosen from the following groups:
halogen groups,
hydroxy,
thiol,
amino,
cyano, —OC(O)C₁-C₆ linear, branched, and cyclic alkyl groups,
—C(O)OC₁-C₆ linear, branched, and cyclic alkyl groups,
—NHC₁-C₆ linear, branched, and cyclic alkyl groups,
—N(C₁-C₆ linear, branched, and cyclic alkyl groups)₂,
—NHC(O)C₁-C₆ linear, branched, and cyclic alkyl groups,
—C(O)NHC₁-C₆ linear, branched, and cyclic alkyl groups,
—NHaryl groups,
—N(aryl groups)₂,
—NHC(O)aryl groups,
—C(O)NHaryl groups,
—NHheteroaryl groups,
—N(heteroaryl groups)₂,
—NHC(O)heteroaryl groups,
—C(O)NHheteroaryl groups,
C₁-C₆ linear, branched, and cyclic alkyl groups,
C₂-C₆ linear, branched, and cyclic alkenyl groups,
C₁-C₆ linear, branched, and cyclic hydroxyalkyl groups,
C₁-C₆ linear, branched, and cyclic alkoxy groups,
C₁-C₆ linear, branched, and cyclic thioalkyl groups,
C₁-C₆ linear, branched, and cyclic haloalkyl groups,
C₁-C₆ linear, branched, and cyclic haloaminoalkyl groups,
C₁-C₆ linear, branched, and cyclic halothioalkyl groups,
C₁-C₆ linear, branched, and cyclic haloalkoxy groups,
benzyloxy, benzylamino, and benzylthio groups,
3 to 6-membered heterocycloalkenyl groups,
3 to 6-membered heterocyclic groups, and
5 and 6-membered heteroaryl groups.

123. The compound, tautomer, deuterated derivative, or pharmaceutically acceptable salt of claim 121 or 122, wherein R¹ is chosen from cyclic alkyl groups, carbocyclic groups, heterocyclic groups, aryl groups, and heteroaryl groups; R² is an aryl group; and R³ is chosen from linear and branched alkyl groups.

124. The compound, tautomer, deuterated derivative, or pharmaceutically acceptable salt of claim 121 or 122, wherein R¹ is chosen from optionally substituted 5 and 6-membered aryl groups.

125. The compound, tautomer, deuterated derivative, or pharmaceutically acceptable salt of claim 124, wherein R¹ is chosen from 5 and 6-membered aryl groups substituted with at least one halogen group.

126. The compound, tautomer, deuterated derivative, or pharmaceutically acceptable salt of claim 125, wherein R¹ is chosen from 5 and 6-membered aryl groups substituted with at least one fluoro.

127. The compound, tautomer, deuterated derivative, or pharmaceutically acceptable salt of claim 125, wherein R¹ is chosen from 5 and 6-membered aryl groups substituted with at least one chloro.

128. The compound, tautomer, deuterated derivative, or pharmaceutically acceptable salt of claim 121 or 122, wherein R¹ is chosen from optionally substituted 5 and 6-membered heteroaryl groups.

129. The compound, tautomer, deuterated derivative, or pharmaceutically acceptable salt of claim 128, wherein R¹ is chosen from 5 and 6-membered heteroaryl groups substituted with at least one halogen group.

130. The compound, tautomer, deuterated derivative, or pharmaceutically acceptable salt of claim 129, wherein R¹ is chosen from 5 and 6-membered heteroaryl groups substituted with at least one fluoro.

131. The compound, tautomer, deuterated derivative, or pharmaceutically acceptable salt of claim 129, wherein R¹ is chosen from 5 and 6-membered heteroaryl group substituted with at least one chloro.

132. The compound, tautomer, deuterated derivative, or pharmaceutically acceptable salt of claim 121 or 122, wherein R¹ is chosen from optionally substituted 3 to 6-membered cyclic alkyl groups.

133. The compound, tautomer, deuterated derivative, or pharmaceutically acceptable salt of claim 132, wherein R¹ is chosen from 3 to 6-membered cyclic alkyl groups substituted with at least one alkoxy group.

134. The compound, tautomer, deuterated derivative, or pharmaceutically acceptable salt of claim 133, wherein R¹ is chosen from 3 to 6-membered cyclic alkyl groups substituted with at least one methoxy group.

135. The compound, tautomer, deuterated derivative, or pharmaceutically acceptable salt of claim 121 or 122, wherein R¹ is chosen from optionally substituted 3 to 6-membered carbocyclic or heterocyclic groups.

136. The compound, tautomer, deuterated derivative, or pharmaceutically acceptable salt of any one of claims 121 to 135, wherein R² is chosen from optionally substituted 5 and 6-membered heteroaryl groups.

137. The compound, tautomer, deuterated derivative, or pharmaceutically acceptable salt of any one of claims 121 to 136, wherein R² is chosen from 5 and 6-membered heteroaryl groups substituted with at least one halogen group.

138. The compound, tautomer, deuterated derivative, or pharmaceutically acceptable salt of claim 137, wherein R² is chosen from 5 and 6-membered heteroaryl groups substituted with at least one fluoro.

139. The compound, tautomer, deuterated derivative, or pharmaceutically acceptable salt of claim 137, wherein R² is chosen from 5 and 6-membered heteroaryl groups substituted with at least one chloro.

140. The compound, tautomer, deuterated derivative, or pharmaceutically acceptable salt of any one of claims 121 to 136, wherein R² is chosen from 5 and 6-membered heteroaryl group substituted with at least one cyano.

141. The compound, tautomer, deuterated derivative, or pharmaceutically acceptable salt of any one of claims 121 to 135, wherein R² is chosen from 5 and 6-membered heteroaryl groups substituted with at least one group chosen from C₁-C₆ linear, C₃-C₆ branched, and C₃-C₆ cyclic alkyl groups.

142. The compound, tautomer, deuterated derivative, or pharmaceutically acceptable salt of any one of claims 121 to 141, wherein R³ is chosen from optionally substituted C₁-C₁₀ linear and C₂-C₁₀ branched alkyl groups.

143. The compound, tautomer, deuterated derivative, or pharmaceutically acceptable salt of claim 142, wherein R³ is chosen from methyl, ethyl, propyl, isopropyl, butyl, iso-butyl, sec-butyl, pentyl, iso-pentyl, sec-pentyl, neo-pentyl, and 1,2,2-trimethylpropyl.

144. The compound, tautomer, deuterated derivative, or pharmaceutically acceptable salt of any one of claims 121 to 143, wherein R⁴ is chosen from optionally substituted C₁-C₁₀ linear, C₂-C₁₀ branched alkyl groups, and C₃-C₆ cyclic alkyl groups.

145. The compound, tautomer, deuterated derivative, or pharmaceutically acceptable salt of claim 144, wherein R⁴ is chosen from methyl, ethyl, propyl, isopropyl, cyclopropyl, butyl, iso-butyl, sec-butyl, cyclobutyl, pentyl, iso-pentyl, sec-pentyl, neo-pentyl, 1,2,2-trimethylpropyl, cyclopentyl, and cyclohexyl.

146. A compound chosen from

| | |
|---|---|
| 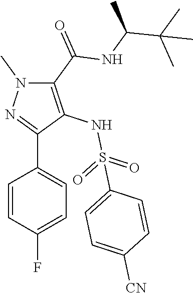 [SMILES: C[C@H](NC(=O)c1c(NS(=O)(=O)c2ccc(C#N)cc2)c(-c2ccc(F)cc2)nn1C)C(C)(C)C] | 1 |
| 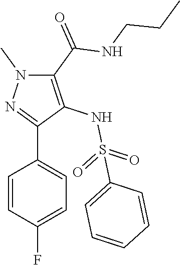 [SMILES: CCCNC(=O)c1c(NS(=O)(=O)c2ccccc2)c(-c2ccc(F)cc2)nn1C] | 2 |
| 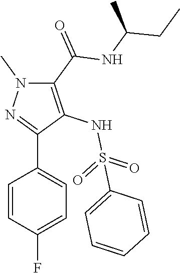 [SMILES: CC[C@H](C)NC(=O)c1c(NS(=O)(=O)c2ccccc2)c(-c2ccc(F)cc2)nn1C] | 3 |
| 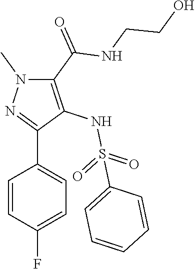 [SMILES: Cn1nc(-c2ccc(F)cc2)c(NS(=O)(=O)c2ccccc2)c1C(=O)NCCO] | 4 |
| 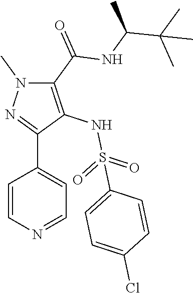 [SMILES: C[C@H](NC(=O)c1c(NS(=O)(=O)c2ccc(Cl)cc2)c(-c2ccncc2)nn1C)C(C)(C)C] | 5 |
| 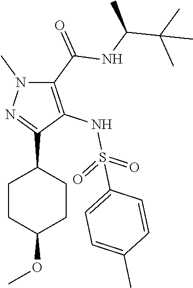 [SMILES: CO[C@H]1CC[C@@H](c2nn(C)c(C(=O)N[C@@H](C)C(C)(C)C)c2NS(=O)(=O)c2ccc(C)cc2)CC1] | 6 |
| 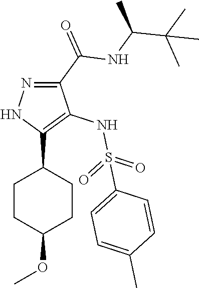 [SMILES: CO[C@H]1CC[C@@H](c2[nH]nc(C(=O)N[C@@H](C)C(C)(C)C)c2NS(=O)(=O)c2ccc(C)cc2)CC1] | 7 |
| 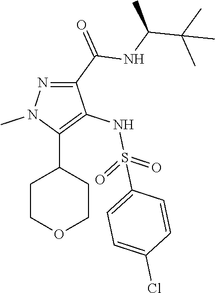 [SMILES: C[C@H](NC(=O)c1nn(C)c(C2CCOCC2)c1NS(=O)(=O)c1ccc(Cl)cc1)C(C)(C)C] | 8 |
| 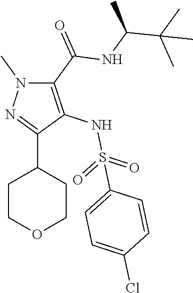 [SMILES: C[C@H](NC(=O)c1c(NS(=O)(=O)c2ccc(Cl)cc2)c(C2CCOCC2)nn1C)C(C)(C)C] | 9 |
| 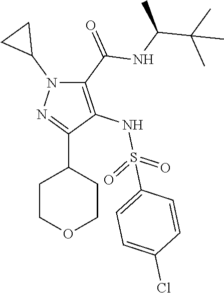 [SMILES: C[C@H](NC(=O)c1c(NS(=O)(=O)c2ccc(Cl)cc2)c(C2CCOCC2)nn1C1CC1)C(C)(C)C] | 10 |
| 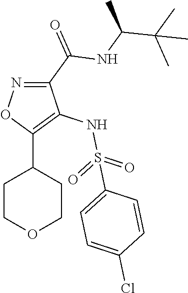 [SMILES: C[C@H](NC(=O)c1noc(C2CCOCC2)c1NS(=O)(=O)c1ccc(Cl)cc1)C(C)(C)C] | 11 |
| 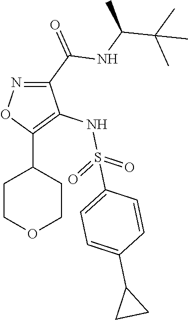 [SMILES: C[C@H](NC(=O)c1noc(C2CCOCC2)c1NS(=O)(=O)c1ccc(C2CC2)cc1)C(C)(C)C] | 12 |
| 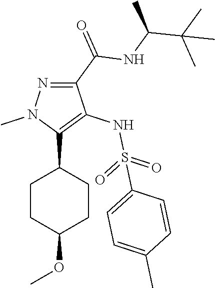 [SMILES: CO[C@H]1CC[C@@H](c2c(NS(=O)(=O)c3ccc(C)cc3)c(C(=O)N[C@@H](C)C(C)(C)C)nn2C)CC1] | 13 |
| 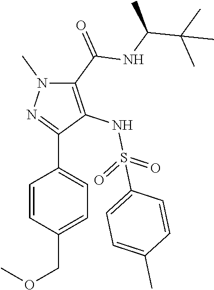 [SMILES: COCc1ccc(-c2nn(C)c(C(=O)N[C@@H](C)C(C)(C)C)c2NS(=O)(=O)c2ccc(C)cc2)cc1] | 14 |
| 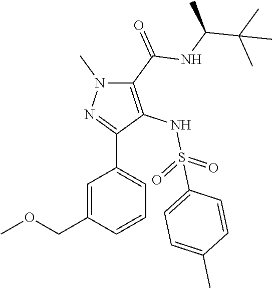 [SMILES: COCc1cccc(-c2nn(C)c(C(=O)N[C@@H](C)C(C)(C)C)c2NS(=O)(=O)c2ccc(C)cc2)c1] | 15 |
| 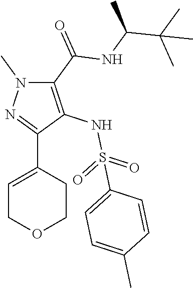 [SMILES: Cc1ccc(S(=O)(=O)Nc2c(C3=CCOCC3)nn(C)c2C(=O)N[C@@H](C)C(C)(C)C)cc1] | 16 |
| 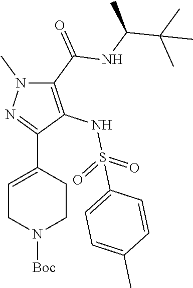 [SMILES: Cc1ccc(S(=O)(=O)Nc2c(C3=CCN(C(=O)OC(C)(C)C)CC3)nn(C)c2C(=O)N[C@@H](C)C(C)(C)C)cc1] | 17 |
| 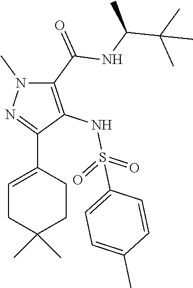 [SMILES: Cc1ccc(S(=O)(=O)Nc2c(C3=CCC(C)(C)CC3)nn(C)c2C(=O)N[C@@H](C)C(C)(C)C)cc1] | 18 |
| 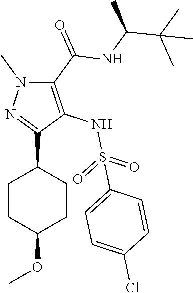 [SMILES: CO[C@H]1CC[C@@H](c2nn(C)c(C(=O)N[C@@H](C)C(C)(C)C)c2NS(=O)(=O)c2ccc(Cl)cc2)CC1] | 19 |
| 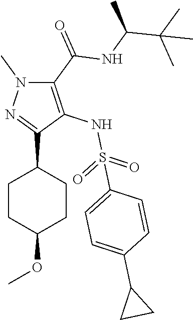 [SMILES: CO[C@H]1CC[C@@H](c2nn(C)c(C(=O)N[C@@H](C)C(C)(C)C)c2NS(=O)(=O)c2ccc(C3CC3)cc2)CC1] | 20 |
| 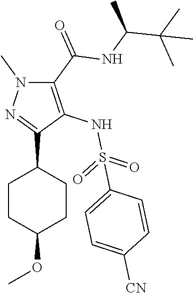 [SMILES: CO[C@H]1CC[C@@H](c2nn(C)c(C(=O)N[C@@H](C)C(C)(C)C)c2NS(=O)(=O)c2ccc(C#N)cc2)CC1] | 21 |
| 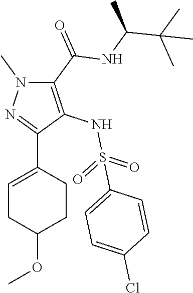 [SMILES: COC1CC=C(c2nn(C)c(C(=O)N[C@@H](C)C(C)(C)C)c2NS(=O)(=O)c2ccc(Cl)cc2)CC1] | 22 |
| 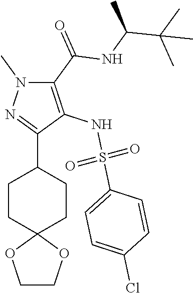 [SMILES: C[C@H](NC(=O)c1c(NS(=O)(=O)c2ccc(Cl)cc2)c(C2CCC3(CC2)OCCO3)nn1C)C(C)(C)C] | 23 |
| 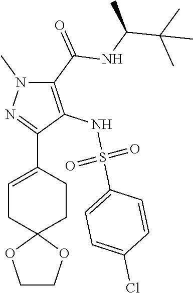 [SMILES: C[C@H](NC(=O)c1c(NS(=O)(=O)c2ccc(Cl)cc2)c(C2=CCC3(CC2)OCCO3)nn1C)C(C)(C)C] | 24 |
| 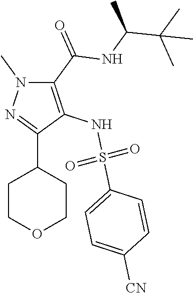 [SMILES: C[C@H](NC(=O)c1c(NS(=O)(=O)c2ccc(C#N)cc2)c(C2CCOCC2)nn1C)C(C)(C)C] | 25 |
| 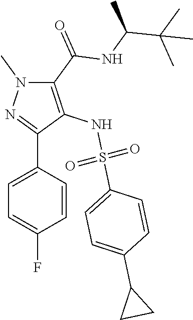 [SMILES: C[C@H](NC(=O)c1c(NS(=O)(=O)c2ccc(C3CC3)cc2)c(-c2ccc(F)cc2)nn1C)C(C)(C)C] | 26 |
| 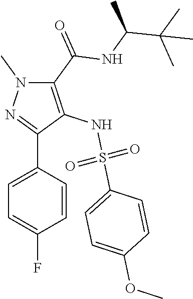 [SMILES: COc1ccc(S(=O)(=O)Nc2c(-c3ccc(F)cc3)nn(C)c2C(=O)N[C@@H](C)C(C)(C)C)cc1] | 27 |
| 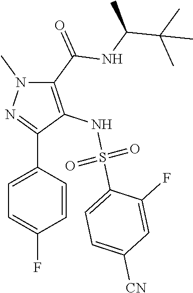 [SMILES: C[C@H](NC(=O)c1c(NS(=O)(=O)c2ccc(C#N)cc2F)c(-c2ccc(F)cc2)nn1C)C(C)(C)C] | 28 |
| 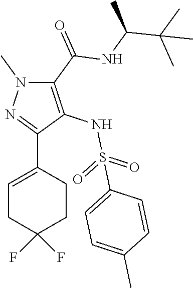 [SMILES: Cc1ccc(S(=O)(=O)Nc2c(C3=CCC(F)(F)CC3)nn(C)c2C(=O)N[C@@H](C)C(C)(C)C)cc1] | 29 |
| 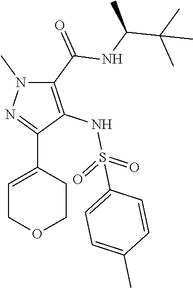 [SMILES: Cc1ccc(S(=O)(=O)Nc2c(C3=CCOCC3)nn(C)c2C(=O)N[C@@H](C)C(C)(C)C)cc1] | 30 |
| 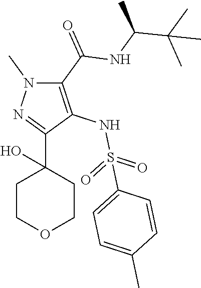 [SMILES: Cc1ccc(S(=O)(=O)Nc2c(C3(O)CCOCC3)nn(C)c2C(=O)N[C@@H](C)C(C)(C)C)cc1] | 31 |
| 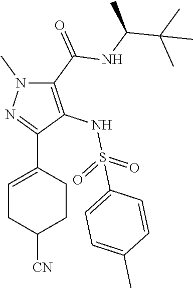 [SMILES: Cc1ccc(S(=O)(=O)Nc2c(C3=CCC(C#N)CC3)nn(C)c2C(=O)N[C@@H](C)C(C)(C)C)cc1] | 32 |
| 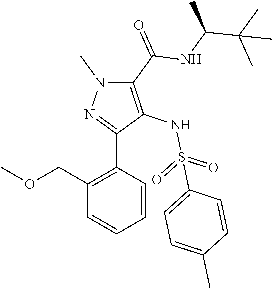 [SMILES: COCc1ccccc1-c1nn(C)c(C(=O)N[C@@H](C)C(C)(C)C)c1NS(=O)(=O)c1ccc(C)cc1] | 33 |
| 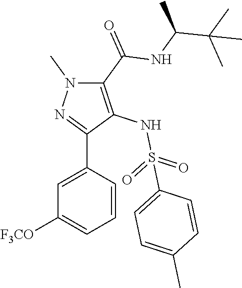 [SMILES: Cc1ccc(S(=O)(=O)Nc2c(-c3cccc(OC(F)(F)F)c3)nn(C)c2C(=O)N[C@@H](C)C(C)(C)C)cc1] | 34 |
| 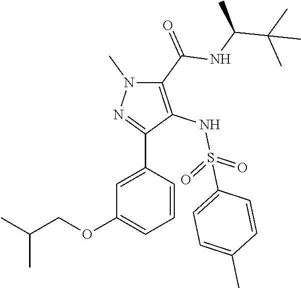 [SMILES: Cc1ccc(S(=O)(=O)Nc2c(-c3cccc(OCC(C)C)c3)nn(C)c2C(=O)N[C@@H](C)C(C)(C)C)cc1] | 35 |
| 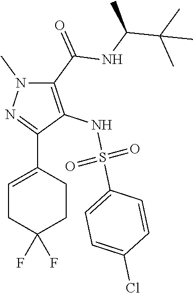 [SMILES: C[C@H](NC(=O)c1c(NS(=O)(=O)c2ccc(Cl)cc2)c(C2=CCC(F)(F)CC2)nn1C)C(C)(C)C] | 36 |
| 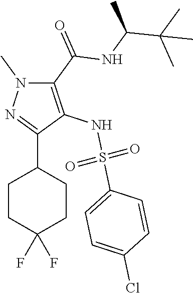 [SMILES: C[C@H](NC(=O)c1c(NS(=O)(=O)c2ccc(Cl)cc2)c(C2CCC(F)(F)CC2)nn1C)C(C)(C)C] | 37 |
| 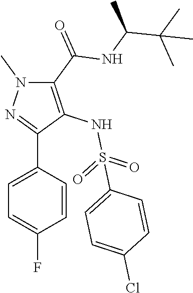 [SMILES: C[C@H](NC(=O)c1c(NS(=O)(=O)c2ccc(Cl)cc2)c(-c2ccc(F)cc2)nn1C)C(C)(C)C] | 38 |
| 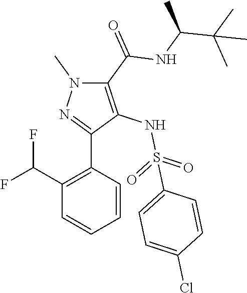 [SMILES: C[C@H](NC(=O)c1c(NS(=O)(=O)c2ccc(Cl)cc2)c(-c2ccccc2C(F)F)nn1C)C(C)(C)C] | 39 |
| 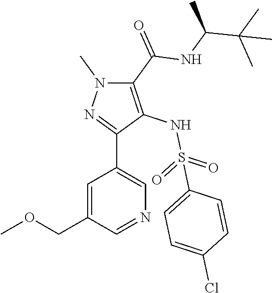 [SMILES: COCc1cncc(-c2nn(C)c(C(=O)N[C@@H](C)C(C)(C)C)c2NS(=O)(=O)c2ccc(Cl)cc2)c1] | 40 |
| 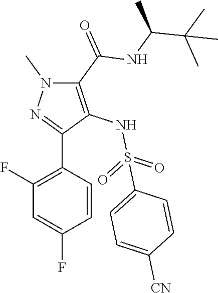 [SMILES: C[C@H](NC(=O)c1c(NS(=O)(=O)c2ccc(C#N)cc2)c(-c2ccc(F)cc2F)nn1C)C(C)(C)C] | 41 |
| 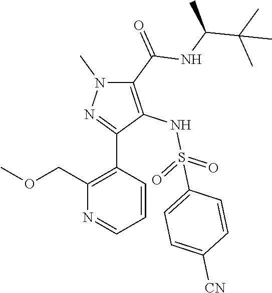 [SMILES: COCc1ncccc1-c1nn(C)c(C(=O)N[C@@H](C)C(C)(C)C)c1NS(=O)(=O)c1ccc(C#N)cc1] | 42 |
| 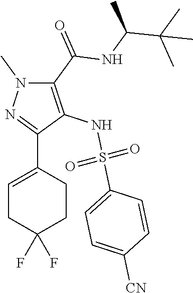 [SMILES: C[C@H](NC(=O)c1c(NS(=O)(=O)c2ccc(C#N)cc2)c(C2=CCC(F)(F)CC2)nn1C)C(C)(C)C] | 43 |
| 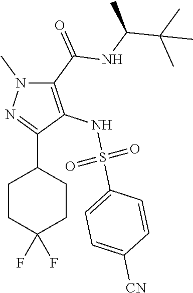 [SMILES: C[C@H](NC(=O)c1c(NS(=O)(=O)c2ccc(C#N)cc2)c(C2CCC(F)(F)CC2)nn1C)C(C)(C)C] | 44 |

a tautomer thereof, a deuterated derivative of the compound or the tautomer, or a pharmaceutically acceptable salt of the foregoing.

147. A pharmaceutical composition comprising a compound, tautomer, deuterated derivative, or pharmaceutically acceptable salt according to any one of claims 1-146 and at least one pharmaceutically acceptable carrier.

148. A method for treating or alleviating a disease, a disorder or a condition mediated by the signaling of formyl peptide receptor 1 (FPR1), comprising administering to a subject in need thereof a therapeutically effective amount of a compound, tautomer, deuterated derivative, or pharmaceutically acceptable salt according to any one of the claims 1-146 or the pharmaceutical composition according to claim 147.

149. The method of claim 148, wherein the disease, the disorder, or the condition is related to the CNS and is chosen from stroke, dementia, Alzheimer's disease, Parkinson's disease, Picks disease, frontotemporal dementia, vascular dementia, normal pressure hydrocephalus, epilepsy, seizure disorder, amyotrophic lateral sclerosis (ALS), spinal motor atrophies, Tay-Sachs disease, Sandhoff disease, familial spastic paraplegia, spinocerebellar ataxia (SCA), Friedrich's ataxia, Wilson's disease, Menkes syndrome, cerebral autosomal dominant arteriopathy with subcortical infarcts (CADASIL), spinal muscular atrophy, muscular dystrophies, Charcot-Marie-Tooth disease, neurofibromatosis, Von Hippel-Lindau disease, Fragile X syndrome, spastic paraplegia, tuberous sclerosis, Waardenburg syndrome, dystonia, benign essential tremor, tardive dystonia, tardive dyskinesia, Tourette's syndrome, ataxic syndromes, Shy Drager syndrome, olivopontocerebellar degeneration, striatonigral degeneration, Guillain-Barre syndrome, causalgia, complex regional pain syndrome types I and II, diabetic neuropathy, and alcoholic neuropathy, trigeminal neuropathy, trigeminal neuralgia, Meniere's syndrome, glossopharyngeal neuralgia, dysphagia, dysphonia, cranial nerve palsies, myelopathy, traumatic brain injury, traumatic spinal injury, radiation brain injury, multiple sclerosis, post-meningitis syndrome, prion diseases, myelitis, radiculitis, diabetes associated with dysproteinemias, transthyretin-induced neuropathies, neuropathy associated with HIV, neuropathy associated with Lyme disease, neuropathy associated with herpes zoster, carpal tunnel syndrome, tarsal tunnel syndrome, amyloid-induced neuropathies, leprous neuropathy, Bell's palsy, compression neuropathies, sarcoidosis-induced neuropathy, polyneuritis cranialis, heavy metal induced neuropathy, transition metal-induced neuropathy, drug-induced neuropathy, axonic brain damage, encephalopathies, chronic fatigue syndrome, and a malignant glioma.

150. The method of claim 148 or 149, wherein the disease, the disorder, or the condition is stroke (thrombotic, embolic, thromboembolic, hemorrhagic, venoconstrictive, and venous).

151. The method of claim 148 or 149, wherein the disease, the disorder, or the condition is traumatic brain injury.

152. The method of claim 148 or 149, wherein the disease, the disorder, or the condition is a malignant glioma.

153. The method of claim 152, wherein the malignant glioma is chosen from glioblastoma, anaplastic astrocytoma, anaplastic oligodendroglioma anaplastic oligoastrocytoma, anaplastic ependymoma, and anaplastic ganglioglioma.

154. The method of claim 153, wherein the malignant glioma is glioblastoma.

155. The method of claim 148, wherein the disease, the disorder, or the condition is chosen from acute respiratory distress syndrome, dry eye syndrome, and allergic conjunctivitis.
